(19) United States
(12) Patent Application Publication
(10) Pub. No.: US 2021/0292417 A1
(43) Pub. Date: Sep. 23, 2021

(54) HISTAMINE DIHYDROCHLORIDE COMBINATIONS AND USES THEREOF

(71) Applicant: Immune Pharmaceuticals LTD., Jerusalem (IL)

(72) Inventors: Anna MARTNER, Gothenburg (SE); Frida EWALD SANDER, Gothenburg (SE); Fredrik BERGH THOREN, Gothenburg (SE); Kristoffer HELLSTRAND, Gothenburg (SE); Hanna WIKTORIN GRAUERS, GOTHENBURG (SE)

(73) Assignee: Immune Pharmaceuticals LTD., Jerusalem (IL)

(21) Appl. No.: 16/075,495

(22) PCT Filed: Feb. 8, 2017
(86) PCT No.: PCT/IL2017/050160

§ 371 Date: Aug. 3, 2018

Related U.S. Application Data
(60) Provisional application No. 62/292,397, filed on Feb. 8, 2016.

(51) Int. Cl. C07K 16/28; A61K 31/417; A61K 45/06; A61P 35/02

(52) U.S. Cl. CPC C07K 16/2827; A61K 31/417; A61K 2039/507; C07K 16/2818; A61P 35/02; A61K 45/06

(57) ABSTRACT

The present invention provides methods of treating cancer in a subject, preventing or delaying relapse to a cancer in a subject in remission, prolonging remission from cancer, increasing survival, and decreasing or alleviating cancer symptoms comprising a) administering histamine dihydrochloride and an inhibitor of the Programmed cell Death protein 1 (PD-1)/Programmed Death Ligand 1 (PD-L1) or b) administering an agent that decreases reactive oxygen species (ROS) optionally, together with a histamine receptor agonist. The present invention also provides methods of predicting the efficacy of a cancer treatment based on a re-distribution of cytotoxic T cells, frequency of NK cells, or other biochemical changes, and related methods of preventing relapse to cancer and for prolonging remission from a cancer. Related kits and compositions are also provided.

### FIELD OF THE INVENTION

The present invention provides methods of treating cancer in a subject, preventing or delaying relapse to a cancer in a subject in remission, prolonging remission from cancer, increasing survival, and decreasing or alleviating cancer symptoms comprising a) administering histamine dihydrochloride and an inhibitor of the Programmed cell Death protein 1 (PD-1)/Programmed Death Ligand 1 (PD-L1) or b) administering an agent that decreases reactive oxygen species (ROS) optionally, together with a histamine receptor agonist. The present invention also provides methods of predicting the efficacy of a cancer treatment based on a re-distribution of cytotoxic T cells, frequency of NK cells, or other biochemical changes, and related methods of preventing relapse to cancer and for prolonging remission from a cancer. Related kits and compositions are also provided.

### BACKGROUND OF THE INVENTION

Histamine dihydrochloride is derived from the biogenic amine histamine. It suppresses the production of reactive oxygen species which inhibits the functions of T cells and natural killer (NK) cells, including their responsiveness to immune activating cytokines. Co-administration of the cytokine interleukin (IL)-2 and histamine dihydrochloride assists the activation of T cells and NK cells by IL-2, leading to the destruction of cancer cells, including those of acute myeloid leukemia (AML).

Patients diagnosed with acute myeloid leukemia (AML) receive induction chemotherapy aiming at attaining the microscopic disappearance of leukemic cells and the re-appearance of normal hematopoiesis (complete remission, CR). The post-remission phase includes consolidation chemotherapy with the goal of eradicating undetectable leukemic cells. However, relapse in CR is common, in particular in older patients (>60 years old), and significantly explains why only a minority of adult AML patients achieve long-term leukemia-free survival (LFS). The prospect of long-term survival after relapse of AML is poor. Therefore, a method of preventing relapse of AML in CR and other cancers is critically needed.

In addition, researchers are aware that cancer treatments are frequently not optimally effective because although the immune system may recognize tumor cells, it may nonetheless remain quiescent due to inhibitory mechanisms which limit or shut down the anti-tumor response. For example, negative regulatory T cell surface molecules were discovered that are upregulated in activated T cells to dampen their activity, resulting in less effective killing of tumor cells. These inhibitory molecules were termed negative co-stimulatory molecules due to their homology to the T cell co-stimulatory molecule CD28. These proteins, also referred to as immune checkpoint proteins, function in multiple pathways including the attenuation of early activation signals, competition for positive co-stimulation and direct inhibition of antigen presenting cells.

One immune checkpoint protein, PD-1 (Programmed cell death protein 1) also known as CD279 (cluster of differentiation 279), is expressed on activated T and B cells as well as on monocytes involved in regulating the balance between immune activation and tolerance. The major role of PD-1 is presumably to limit the activity of T cells in the periphery during an inflammatory response to infection and to limit autoimmunity. PD-1 ligands B7-H1/PD-L1 and B7-DC/PD-L2 are upregulated in response to various proinflammatory cytokines and can bind to PD-1 on activated T cells in inflamed tissues, thereby limiting the immune response.

PD-1 ligands are expressed in higher than normal levels on many human tumors, such as carcinomas of the lung, ovary and colon, and melanomas, where they inhibit local anti-tumor T cell responses by binding to PD-1 on tumor infiltrating lymphocytes. Inhibition of the interaction between PD-1 and PD-L1 can enhance T-cell responses in vitro and mediate preclinical antitumor activity.

The use of immune checkpoint inhibitors (ICIs), such as PD-1, appears to represent a promising approach for improved cancer immunotherapy. However, the combination of PD-1/PD-L1 inhibitors with other means of immune activation, including vaccines, often did not lead to the hoped improvement of the immunotherapy.

Therefore, a safe and effective means for a therapy based on treatment with ICIs, particularly based on PD-1 pathway inhibitors, is needed, in particular for a therapy of tumor, cancer and/or infectious diseases.

### SUMMARY OF THE INVENTION

In one embodiment, the present invention provides a method of reducing the tumor burden in a subject with primary or metastatic cancer comprising the step of: administering a therapeutic amount of histamine dihydrochloride and inhibitors of Programmed cell Death protein 1 (PD-1) or Programmed cell Death Ligand 1 (PD-L1) to said subject, thereby reducing the tumor burden in said subject.

In another embodiment, the present invention provides a method of reducing the risk of metastatic tumor spread in a subject with active cancer comprising the step of: administering a therapeutic amount of histamine dihydrochloride and inhibitors of Programmed cell Death protein 1 (PD-1) or Programmed cell Death Ligand 1 (PD-L1) to said subject, thereby reducing the risk of metastatic tumor spread in said subject.

In another embodiment, the present invention provides a method of preventing or delaying the reappearance, recurrence or metastatic spread of cancer in a subject comprising the step of: administering a therapeutic amount of histamine dihydrochloride and inhibitors of Programmed cell Death protein 1 (PD-1) or Programmed cell Death Ligand 1 (PD-L1) to said subject, thereby preventing or delaying the reappearance, recurrence or metastatic spread of said cancer in said subject.

In another embodiment, the present invention provides a method of preventing relapse to a cancer in a subject in remission from said cancer comprising the step of: administering a therapeutic amount of a histamine receptor agonist and Programmed cell Death protein 1 (PD-1) or Programmed cell Death Ligand 1 (PD-L1) inhibitors to said subject, thereby preventing relapse to said cancer in said subject in remission from said cancer.

In another embodiment, the present invention provides a method of delaying the relapse to a cancer in a subject in remission from said cancer comprising the step of: administering a therapeutic amount of a histamine receptor agonist and Programmed cell Death protein 1 (PD-1) or Programmed cell Death Ligand 1 (PD-L1) inhibitors to said subject, thereby delaying the relapse to said cancer in said subject in remission from said cancer.

In another embodiment, the present invention provides a method of prolonging the remission from a cancer in a subject comprising the step of: administering a therapeutic amount of a histamine receptor agonist and Programmed cell Death protein 1 (PD-1) or Programmed cell Death Ligand 1 (PD-L1) inhibitors to said subject, thereby prolonging the remission from said cancer in said subject.

In another embodiment, the present invention provides a method of increasing the survival of a subject in remission from a cancer comprising the step of: administering a therapeutic amount of a histamine receptor agonist and Programmed cell Death protein 1 (PD-1) or Programmed cell Death Ligand 1 (PD-L1) inhibitors to said subject, thereby increasing the survival of said subject.

In another embodiment, the present invention provides a method of prolonging the survival time of a subject in remission from a cancer comprising the step of: administering a therapeutic amount of a histamine receptor agonist and inhibitors of Programmed cell Death protein 1 (PD-1) or Programmed cell Death Ligand 1 (PD-L1) to said subject, thereby prolonging the survival time of said subject.

In another embodiment, the present invention provides a method of reducing malignant tumor growth in a subject comprising the step of: administering a therapeutic amount of a histamine receptor agonist and Programmed cell Death protein 1 (PD-1) or Programmed cell Death Ligand 1 (PD-L1) inhibitors to said subject, thereby reducing malignant tumor growth in said subject.

In another embodiment, the present invention provides a method of decreasing or alleviating cancer symptoms in a subject in remission from a cancer comprising the step of: administering a therapeutic amount of a histamine receptor agonist and Programmed cell Death protein 1 (PD-1) or Programmed cell Death Ligand 1 (PD-L1) inhibitors to said subject, thereby decreasing or alleviating said cancer symptoms in said subject.

In another embodiment, the present invention provides a kit for prolonging remission from a cancer in a subject comprising a) a therapeutic amount of a histamine receptor agonist and Programmed cell Death protein 1 (PD-1) or Programmed cell Death Ligand 1 (PD-L1) inhibitors, and instructions for the use of said kit.

In another embodiment, the present invention provides a method of predicting the efficacy of a cancer treatment in a subject comprising the steps of: a) obtaining a first blood sample from said subject; b) administering a cycle of a treatment or a proposed treatment for said cancer to said subject; c) obtaining a second blood sample from said subject after completion of said cycle of treatment; and d) measuring the frequency of CD8+ cytotoxic T cell phenotypes in said first blood sample and said second blood sample, wherein if there is a re-distribution of cytotoxic T cells such that there is a reduction in the frequency of T effector memory cells (T_{EM}) and an increase in the frequency of T effector cells (T_{eff}) in said second blood sample compared to said first blood sample, then said cancer treatment is predicted to be effective in said subject and wherein if there is no re-distribution of cytotoxic T cells in said second blood sample compared to said first blood sample, then said cancer treatment is predicted not to be effective in said subject, thereby predicting the efficacy of said cancer treatment in said subject.

In another embodiment, the present invention provides a method of preventing relapse to a cancer in a subject in remission from said cancer comprising the steps of: a) obtaining a first blood sample from said subject; b) administering a cycle of a treatment or a proposed treatment for said cancer to said subject; c) obtaining a second blood sample from said subject after completion of said cycle of treatment; d) measuring the frequency of CD8⁺ cytotoxic T cell phenotypes in said first blood sample and said second blood sample, and e) administering additional cycles of said treatment to said subject if there is a re-distribution of cytotoxic T cells such that there is a reduction in the frequency of T effector memory cells (T_{EM}) and an increase in the frequency of T effector cells (T_{eff}) in said second blood sample compared to said first blood sample, thereby preventing relapse to said cancer in said subject.

In another embodiment, the present invention provides a method of prolonging remission from a cancer in a subject comprising the steps of: a) obtaining a first blood sample from said subject; b) administering a cycle of a treatment or a proposed treatment for said cancer to said subject; c) obtaining a second blood sample from said subject after completion of said cycle of treatment; d) measuring the frequency of CD8+ cytotoxic T cell phenotypes in said first blood sample and said second blood sample, and e) administering additional cycles of said treatment to said subject if there is a re-distribution of cytotoxic T cells such that there is a reduction in the frequency of T effector memory cells (T_{EM}) and an increase in the frequency of T effector cells (T_{eff}) in said second blood sample compared to said first blood sample, thereby prolonging remission from said cancer in said subject.

In another embodiment, the present invention provides a method of preventing relapse to acute myeloid leukemia (AML) in a subject with AML in complete remission (CR) after chemotherapy comprising the steps of: a) obtaining a first blood sample from said subject; b) administering a first cycle of histamine dihydrochloride and interleukin-2 (IL-2) to said subject; c) obtaining a second blood sample from said subject after completion of said first cycle of treatment; d) measuring the distribution of CD8⁺ cytotoxic T cell phenotypes in said blood sample; and e) administering additional cycles of histamine dihydrochloride and IL-2 to said subject if there is a re-distribution of cytotoxic T cells in said second blood sample compared to said first blood sample such that there is a reduction in the frequency of T effector memory cells (T_{EM}) and an increase in the frequency of T effector cells (T_{eff}), thereby preventing relapse to AML in said subject.

In another embodiment, the present invention provides a kit for predicting the efficacy of a cancer treatment in a subject comprising a therapeutic amount of a histamine receptor agonist, an immunostimulant, a means for measuring CD8+ cytotoxic phenotypes, and instructions for the use of said kit.

In another embodiment, the present invention provides a method of predicting the efficacy of a cancer treatment in a subject comprising the steps of: (a) obtaining a first blood sample from said subject; (b) administering a cycle of a treatment or a proposed treatment for said cancer to said subject; (c) obtaining a second blood sample from said subject after completion of said cycle of treatment; and (d) measuring frequency of NK cells in said first blood sample and said second blood sample, wherein if there is an increase in the frequency of NK cells in said second blood sample compared to said first blood sample, then said cancer treatment is predicted to be effective in said subject and wherein if there is no increase in the frequency of NK cells in said second blood sample compared to said first blood sample, then said cancer treatment is predicted not to be effective in said subject, thereby predicting the efficacy of said cancer treatment in said subject.

In another embodiment, the present invention provides a method of predicting the efficacy of a cancer treatment in a subject comprising the steps of: (a) obtaining a first blood sample from said subject; (b) administering a cycle of a treatment or a proposed treatment for said cancer to said subject; (c) obtaining a second blood sample from said subject after completion of said cycle of treatment; and (d) measuring the levels of a biomarker expression in said first blood sample and said second blood sample, wherein if there is an increase in the biomarker expression level in said second blood sample compared to said first blood sample, then said cancer treatment is predicted to be effective in said subject and wherein if there is no increase in the biomarker expression level in said second blood sample compared to said first blood sample, then said cancer treatment is predicted to be ineffective in said subject, thereby predicting the efficacy of said cancer treatment in said subject.

In another embodiment, the present invention provides a method of predicting the efficacy of a cancer treatment in a subject comprising the steps of: (a) obtaining a first blood sample from said subject; (b) administering a cycle of a treatment or a proposed treatment for said cancer to said subject; (c) obtaining a second blood sample from said subject after completion of said cycle of treatment; and (d) measuring the levels of a biomarker expression in said first blood sample and said second blood sample, wherein if there is an decrease in the biomarker expression level in said second blood sample compared to said first blood sample, then said cancer treatment is predicted to be effective in said subject and wherein if there is no decrease in the biomarker expression level in said second blood sample compared to said first blood sample, then said cancer treatment is predicted not to be effective in said subject, thereby predicting the efficacy of said cancer treatment in said subject.

In another embodiment, the present invention provides a method of treating a cancer in a subject comprising the step of: administering a therapeutic amount of an agent that decreases reactive oxygen species (ROS) to said subject.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1. Individual Tumor Size of each Subject in the Four Treatment Groups. The tumor size for each subject in control (n=29), histamine (n=29), α-PD1/PDL1 (n=8), and histamine+α-PD1/PDL1 (n=8) treatment groups on days 8, 11, and 13 during tumor progression is shown. Tumor size is indicated as % of control, where 100% was the mean size of control tumors at 2 weeks after tumor inoculation.

FIG. 2. Mean Tumor Size in the Four Treatment Groups. The mean±standard error of the mean (s. e. m.) tumor size in control (n=29), histamine (n=29), α-PD1/PDL1 (n=8), and histamine+α-PD1/PDL1 (n=8) treatment groups on days 8, 11, and 13 during tumor progression is shown. The groups were compared statistically using a two-way ANOVA. *p<0.05, ***p<0.001. All indicated p-values are two-sided. Tumor size is indicated as % of control, where 100% was the mean size of control tumors at 2 weeks after tumor inoculation.

FIG. 3. Tumor Size in the Four Treatment Groups. The size of the tumors in the four treatment groups are shown as box plots at the respective time-points of tumor measurement. The boxes shown the 25^{th} and 75^{th} quartiles of tumor size with the median tumor size indicated by a horizontal line. The whiskers indicate the highest and lower values of tumor size. P-values were calculated using Student's t-test. *p<0.05, **p<0.01, ***p<0.001. All indicated p-values are two-sided. Tumor size is indicated as % of control, where 100% was the mean size of control tumors at 2 weeks after tumor inoculation.

FIG. 4. Overview of the Re:Mission Phase IV Trial. Eligible AML patients in first complete remission (CR) received ten 3-week cycles of HDC/IL-2 over 18 months. Peripheral blood mononuclear cells (PBMC) were isolated from blood collected before and after cycles 1 and 3. Patients were follow-up for 6 months after completing the last treatment cycle.

FIG. 5. Analysed Patients. Flow chart showing the number of patients included to the study and the number of successfully analysed samples.

FIG. 6. Distribution of CD8⁺ Subsets in Non-Relapsing and Relapsing AML Patients during Immunotherapy with HDC/IL-2. (A) Blood counts of CD8⁺ T cells before (cycle 1, day 1, C1D1) and after (C1D21) the first and third cycles of HDC/IL-2 treatment (C1D1 n=62; C1D21 n=54; C3D1 n=52; C3D21 n=51). (B) Gating strategy for determining naïve (T_{N}; CD45RA⁺CCR7⁺), central memory (T_{CM}; CD45RO⁺CCR7⁺), effector memory (T_{EM}; CD45RO⁺CCR7⁻) and effector (T_{eff}; CD45RA⁺CCR7⁻) cells within the CD8⁺ T cell compartment. (C-F) Frequency of the CD8⁺ subpopulations T_{N} (C), T_{CM} (D), T_{EM} (E) and T_{eff} (F) cells in non-relapsing (n=18) and relapsing (n=26) patients at the onset (C1D1) or end of (C1D21) the first cycle of immunotherapy. Statistical analysis was performed by Student's paired t-test.

FIG. 7. Impact of CD8+ Subsets at the Onset of Immunotherapy on LFS. Patients were dichotomized based on the median percentage of T_{N} (A) T_{CM} (B), T_{EM} (C) and T_{eff} (D) CD8+ T cells in blood samples collected before the first treatment cycle of HDC/IL-2. LFS was analyzed by the logrank test.

FIG. 8. Impact of Altered Distribution of CD8+ Subsets on the Clinical Outcome of Patients Receiving HDC/IL-2. In (A-D) all patients, and in (F-I) patients>60 years old, were dichotomized based on induction or reduction of the frequency of CD8+ T cell subsets during the first treatment cycle, followed by analyses of LFS and OS by the logrank test. In (E) all patients, and in (J) patients >60 years old, were dichotomized based on transition (trans) or no transition from T_{EM} to T_{eff} cells and LFS and OS were analyzed by the logrank test. A patient was considered transition-positive by the occurrence of a reduction of T_{EM} cells (%) and a simultaneous induction of T_{eff} cells (%) during the first treatment cycle.

FIG. 9. Negative Correlation between Changes in the Ddistribution of T_{EM} and T_{eff} cells During Immunotherapy. Changes in the frequency of CD8+T_{EM} (y-axis) and T_{eff} (x-axis) during the first treatment cycle with HDC/IL-2 were correlated. Non-relapsing patients are marked by red dots, while relapsing patients are marked by black dots.

FIG. 10. Distribution of CD8+ T Cell Populations in Younger and Older Patients Receiving HDC/IL-2. (A-D) Frequency of the CD8+ subpopulations T_{N}, T_{CM}, T_{EM} and T_{eff} cells in patients <60 years old (n=17) and patients >60 years old (n=27) before (cycle 1, day 1; C1D1) or after (C1D21) cycle 1 of HDC/IL-2 immunotherapy. Statistical analysis was performed by Student's paired t-test. In (E-L) patients <60 years old were dichotomized based on induction or reduction of the frequency of the different CD8+ subsets during the first treatment cycle, followed by analyses of LFS and OS by the logrank test. In (M-N), patients<60 years old were dichotomized based on transition or no transition from T_{EM} to T_{eff} cells and LFS and OS were analysed by the logrank test.

FIG. 11. Expression of Activation Markers on CD8+ T cells during immunotherapy with HDC/IL-2. Frequency of (A) CD25+ and (B) CD69+ cells within the CD3+CD8+ population (cycle 1, day 1; C1D1 n=62; C1D21 n=63). (C) Median fluorescence intensity (MFI) of HLA-DR on CD3+CD8+ T cells (n=44). (D) Blood samples collected at the onset (C1D1) or end of (C1D21) the first cycle of immunotherapy were stimulated with PMA/ionomycin followed by intracellular staining of IFN-γ. The box plots show the frequency of IFN-γ-producing CD8+ T cells before and after the first treatment cycle (C1D1 n=58; C1D21 n=63). Statistical analysis was performed by Student's paired t-test. The reduction of HLA-DR intensity during cycle 1 (shown in panel C) remained statistically significant in non-parametric analysis (Wilcoxon signed-rank test, P=0.0003).

FIG. 12. Impact of HLA-DR Expression and Leukemia-Specific CD8⁺ T Cells on LFS in Patients Receiving HDC/IL-2. (A) Patients were dichotomized by the median HLA-DR expression on CD3⁺CD8⁺ T cells at onset of therapy (C1D1; n=44) or after the first treatment cycle (C1D21; n=47). LFS and OS were analyzed by the logrank test. (B-C) Blood samples from patients undergoing HDC/IL-2 treatment were stimulated with a pool of peptides from leukemia-associated antigens (AML-peptides) or a pool of peptides from CMV, EBV and influenza viruses (CEF-peptides), or no peptides (negative control). The percentage of IFN-γ producing CD8⁺ T cells was determined by flow cytometry. In (B) representative dot plots show IFN-γ production in samples without stimulation and samples stimulated with AML- or CEF-peptides. In (C) patients were dichotomized based on the presence or absence of AML-specific or CEF-specific CD8⁺ T cells, followed by analysis of LFS by the logrank test. Only patients with no events occurring before the last time point of analysis of antigen-specific T cells (C3D21; 105 days) were considered in the latter analyses.

FIG. 13. Impact of T_{EM} to T_{eff} Cell Transition and NK cell NKp46 Expression on Clinical Outcome. (A-B) Patients were regarded as transition-positive when showing a reduction of T_{EM} cells (%) and a simultaneous induction of T_{eff} cells (%) during the first treatment cycle, and were considered NKp46^{high} when their CD16⁺ NK cells expressed above median levels of NKp46 after the first cycle of immunotherapy (C1D21). Data show the LFS and OS (analyzed by the logrank test for trend) of patients with transition T_{EM}-T_{eff} and NKp46^{high} (both), transition only, NKp46^{high} only, no transition T_{EM}-T_{eff} and low NKp46 expression (neither).

FIG. 14. Induction and Activation of NK Cells in Patients Below and Above 60 Years of Age. The box plots in panel A-F show the number of CD16+ or CD56^{bright} NK cells in blood (A and D). and the median fluorescence intensity (MFI) of NKp30 (B and E). and NKp46 (C and F). on CD16⁺ or CD56^{bright} NK cells before (C1D1) and after (C1D21) the first 21-day treatment cycle with HDC/IL-2 in patients <60 years (young, white; NK cell counts, n=17, NCR expression, n=21) and >60 years (old, grey; NK cell counts, n=30, NCR expression, n=35). Induction of NK cell counts and NCR expression were analyzed using Student's paired t-test.

FIG. 15: Impact of NK Cell NCR Expression on Leukemia-Free Survival (LFS) and Overall Survival (OS) in Older AML Patients. In panels A-D, LFS and OS are shown for older patients (>60), dichotomized based on above (red) or below (black) median of NKp30 (A and C). or NKp46 (B and D). expression on CD16⁺ NK cells before (A and B) or after (C and D) the first HDC/IL-2 treatment cycle.

FIG. 16. Immunotherapy with HDC/IL-2 Increases White Blood Cell Counts and Decreases Blood Monocyte Levels. Panels A-D show peripheral blood counts of (A) white blood cells, (B) eosinophils, (C) neutrophils and (D) monocytes before (day 1; D1) and after (day 21; D21) of cycles 1 (Cl) and 3 (C3) of immunotherapy with HDC/IL-2. Student's paired t-test was employed for analyzing differences between time points. In panels E-F patients were dichotomized based on the median monocyte reduction during cycle 1 followed by analysis of LFS and OS by the logrank test.

FIG. 17. Impact of histamine H2R Expression on LFS and OS. Panels A-B show the expression of H2R of (A) CD14++ and (B) CD16⁺ monocytes during cycles of HDC/IL-2 as analyzed by Student's paired t-test. In panels C-F patients were dichotomized based on the median H2R expression (MFI) of (C and E) CD14++ or (D and F) CD16⁺ monocytes on 1 day 21 (C1D21) of immunotherapy followed by analysis of LFS and OS by the logrank test.

FIG. 18. Redistribution of myeloid Surface Markers during HDC/IL-2 Immunotherapy. Panel A shows the expression of HLA-DR, CD40 and CD86 (MFI) on CD14+ monocytes in cycles 1 and 3 at days 1 and 21. Panels B-D show corresponding expression data for (B) CD16⁺ monocytes, (C) CD1c+ DCs and (D) CD141+ DCs. Student's paired t-test was employed to analyze differences between time points.

FIG. 19. HLA-ABC Expression on myeloid Cells Predicts LFS During Treatment with HDC/IL-2. Panels A-D show the HLA-ABC expression at indicated time points for the following subsets of myeloid cells: (A) CD14+ monocytes, (B) CD16⁺ monocytes, (C) CD1c+ DCs and (D) CD141+ DCs. Student's paired t-test was employed to analyze differences between time points. In panels E (LFS) and F (OS) patients were dichotomized based on the median HLA-ABC expression of CD14+ monocytes on cycle 1 day 1 (C1D1) followed by analysis of LFS and OS by the logrank test.

FIG. 20. Impact of monocyte HLA-ABC Expression on the Outcome of Patients with or without High CD8+ TEM Counts at Onset of Immunotherapy or TEM to TEff Transition During Immunotherapy. In panels A-C, patients with high or low counts of blood CD8+ TEM cells on cycle 1 day 1 (C1D1) were dichotomized based on the median HLA-ABC expression on CD14+ monocytes on C1D1 followed by analysis of LFS or OS by the logrank test. Panels D-F show corresponding results for patients who did or did not achieve transition of CD8+ TEM to TEff cells during cycle 1 of immunotherapy.

FIG. 21. CMML Patients Harbor Leukemic Cells with Immunosuppressive Properties. Panel A shows a representative gating strategy for CMML mononuclear cells in peripheral blood. The green population comprises mature leukemic CD14+ cells whilst immature CD34+ blasts are depicted in blue. (B) Flow cytometry analysis of gp91phox and H2R on CD14+ and CD34+ cells in peripheral blood from CMML patients (n=11) and healthy controls (n=10). Panel C shows a representative ROS measurement from FACS-sorted CD14+ CMML cells with or without inhibition with HDC (50 μM), ranitidine (10 μM) and AH202399AA (10 μM) whilst panel D shows 11 combined experiments using NOX-2 inhibitor HDC (100 μM, p<0.001). Panel D analyzed using Wilcoxon matched-pairs signed rank test.

FIG. 22. ROS-Producing CMML Cells Impair NK Cell Function. Panel A shows a confocal micrograph depicting the interaction between a NK cell (left) and a CD14+ CMML cell (right) is shown. Blue stain represents DAPI staining of the nucleus and green dye is bound to membrane-bound gp91phox. (B) Degranulation against CD14+ CMML cells by IL-2-stimulated NK cells (n=9) in the presence or absence of anti-CD33 antibody lintuzumab (1 μg/ml) and HDC (100 μM). Data were analyzed using Student's paired t-test with Bonferroni's correction. Panel C shows NK cell death (n=6) after over-night incubation with CMML cells at ratios ranging from 16:1 to 1:1 For each donor, the degree of apoptotic NK cells from the first NK:CMML ratio with at least 50% apoptotic NK cells is shown in the figure. When indicated, the NOX2-inhibitor HDC (100 μM), the PARP-1 inhibitor PJ34 (0.5 μM), the NOX2 inhibitor DPI (3 μM) or catalase (200U/ml) was present during the overnight incubation. The effect of the inhibitors on CMML-induced NK cell apoptosis was analyzed by one-way ANOVAs followed by Bonferroni's multiple comparison test.

FIG. 23. CD8+ T Cells are Sensitive to CMML cell Induced ROS-Mediated Cell Death. Panel A shows CD8+ T cell apoptosis (n=4) after overnight incubation with CMML cells at ratio 1:1 in the presence or absence of inhibitors HDC (100 μM), PARP-1 inhibitor PJ34 (0.5 μM), NOX2 inhibitor DPI (3 μM) or catalase (200U/ml). Analyzed by one-way ANOVAs followed by Bonferroni's multiple comparison test. In panel B, indicated CD8+ T cell subsets were sorted and incubated overnight with CMML cells at ratio 1:1 with or without NOX2 inhibitor DPI (3 μM, n=3). TN cells were defined as CD45RA+ CCR7+, T_{CM} as CD45RO+ CCR7+, T_{EM} as CD45RO+ CCR7− and T_{Eff} as CD45RA+ CCR7−. Analyzed by oneway ANOVAs followed by Bonferroni's multiple comparison test.

FIG. 24. Expression of Activating NK Cell Receptors in CMML Patients. Panel A shows median fluorescence intensity (MFI) of receptors NKp30, NKp46, NKp80, NKG2D, DNAM-1 and 2B4 whilst panel B shows percentage of NK cells positive for corresponding receptors. In panel A and B, CMML patients (n=10) were divided into above (n=5) or below (n=5) 2% CD34+ blasts in PBMC and compared to age-matched controls (H.D; n=10). Data was analyzed by Student's t-test.

FIG. 25. ROS-mediated down-regulation of activating receptors. (A) NK cells and monocytes from healthy donors were co-incubated overnight in the presence or absence of PARP-1 inhibitor PJ34 (0.5 μM). Median fluorescence intensities (MFI) for receptors NKp46, NKp80, DNAM-1 and CD16 are displayed. Data analyzed by Student's t-test.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

In another embodiment, the present invention provides a method of treating a cancer in a subject comprising the step of: administering a therapeutic amount of a histamine receptor agonist with another therapeutic agent to said subject, thereby treating said cancer in said subject. In one embodiment, the therapeutic agent is a Programmed cell Death protein 1 (PD-1) inhibitor. In another embodiment, the therapeutic agent is a Programmed cell Death Ligand 1 (PD-L1) inhibitor.

In one embodiment, the present invention provides a method of reducing the tumor burden in a subject with primary or metastatic cancer comprising the step of: administering a therapeutic amount of histamine dihydrochloride and inhibitors of Programmed cell Death protein 1 (PD-1) or Programmed cell Death Ligand 1 (PD-L1) to said subject, thereby reducing the tumor burden in said subject.

In another embodiment, the present invention provides a method of reducing the risk of metastatic tumor spread in a subject with active cancer comprising the step of: administering a therapeutic amount of histamine dihydrochloride and inhibitors of Programmed cell Death protein 1 (PD-1) or Programmed cell Death Ligand 1 (PD-L1) to said subject, thereby reducing the risk of metastatic tumor spread in said subject.

In another embodiment, the present invention provides a method of preventing or delaying the reappearance, recurrence or metastatic spread of cancer in a subject comprising the step of: administering a therapeutic amount of histamine dihydrochloride and inhibitors of Programmed cell Death protein 1 (PD-1) or Programmed cell Death Ligand 1 (PD-L1) to said subject, thereby preventing or delaying the reappearance, recurrence or metastatic spread of said cancer in said subject.

In another embodiment, the present invention provides a method of preventing relapse to a cancer in a subject in remission from said cancer comprising the step of: administering a therapeutic amount of a histamine receptor agonist and Programmed cell Death protein 1 (PD-1) or Programmed cell Death Ligand 1 (PD-L1) inhibitors to said subject, thereby preventing relapse to said cancer in said subject in remission from said cancer.

In another embodiment, the present invention provides a method of delaying the relapse to a cancer in a subject in remission from said cancer comprising the step of: administering a therapeutic amount of a histamine receptor agonist and Programmed cell Death protein 1 (PD-1) or Programmed cell Death Ligand 1 (PD-L1) inhibitors to said subject, thereby delaying the relapse to said cancer in said subject in remission from said cancer.

In another embodiment, the present invention provides a method of prolonging the remission from a cancer in a subject comprising the step of: administering a therapeutic amount of a histamine receptor agonist and Programmed cell Death protein 1 (PD-1) or Programmed cell Death Ligand 1 (PD-L1) inhibitors to said subject, thereby prolonging the remission from said cancer in said subject.

In another embodiment, the present invention provides a method of increasing the survival of a subject in remission from a cancer comprising the step of: administering a therapeutic amount of a histamine receptor agonist and Programmed cell Death protein 1 (PD-1) or Programmed cell Death Ligand 1 (PD-L1) inhibitors to said subject, thereby increasing the survival of said subject.

In another embodiment, the present invention provides a method of prolonging the survival time of a subject in remission from a cancer comprising the step of: administering a therapeutic amount of a histamine receptor agonist and inhibitors of Programmed cell Death protein 1 (PD-1) or Programmed cell Death Ligand 1 (PD-L1) to said subject, thereby prolonging the survival time of said subject.

In another embodiment, the present invention provides a method of reducing malignant tumor growth in a subject comprising the step of: administering a therapeutic amount of a histamine receptor agonist and Programmed cell Death protein 1 (PD-1) or Programmed cell Death Ligand 1 (PD-L1) inhibitors to said subject, thereby reducing malignant tumor growth in said subject.

In another embodiment, the present invention provides a method of decreasing or alleviating cancer symptoms in a subject in remission from a cancer comprising the step of: administering a therapeutic amount of a histamine receptor agonist and Programmed cell Death protein 1 (PD-1) or Programmed cell Death Ligand 1 (PD-L1) inhibitors to said subject, thereby decreasing or alleviating said cancer symptoms in said subject.

In another embodiment, the present invention provides a kit for prolonging remission from a cancer in a subject comprising a) a therapeutic amount of a histamine receptor agonist and Programmed cell Death protein 1 (PD-1) or Programmed cell Death Ligand 1 (PD-L1) inhibitors, and instructions for the use of said kit.

### PD-1 Inhibitors

In one embodiment, a cancer treatment of the present invention comprises administration of an inhibitor of PD-1, in combination with one or more therapeutic agents as described herein. In one embodiment, the PD-1 inhibitor is an antibody. In one embodiment, the antibody is an antagonistic antibody or a nucleic acid-encoded antibody (intrabody). In another embodiment, the PD-1 inhibitor is an siRNA, an antisense RNA, a protein comprising (or a nucleic acid coding for) an amino acid sequence capable of binding to PD-1 but preventing PD-1 signaling (e.g. a fusion protein of a fragment of PD-L1 or PD-L2 and the Fc part of an immunoglobulin), a soluble protein (or a nucleic acid coding for a soluble protein) competing with membrane-bound PD-1 for binding of its ligands PD-L1 and PD-L2; or a small molecule inhibitor capable of inhibiting PD-1 pathway signaling.

In one embodiment, the PD-1 inhibitor is the anti-PD1 antibody Nivolumab (MDX-1 106/BMS-936558/ONO-4538), (Brahmer et al., 2010. J Clin Oncol. 28(19):31 67-75; PMID: 2051 6446); or Pidilizumab (CT-01 1), (Berger et al., 2008. Clin Cancer Res. 14(10):3044-51; PMID: 18483370); and MK-3475 (SCH 900475). In another embodiment, the anti-PD1 antibody is Pembrolizumab.

In another embodiment, the PD-1 pathway inhibitor is an antibody (or a nucleic acid coding for an antibody) directed against a PD-1 ligand, in one embodiment, an antibody specifically binding to the extracellular domain of the PD-1 or PD-2 ligand. In one embodiment, the antibody binds proximal to and disruptive of the PD-1 or PD-2 binding site on the ligand.

### PD-L1 Inhibitors

In one embodiment, the anti-PD-L1 antibody is MDX-1 105/BMS-936559 (Brahmer et al. 2012. N Engl J Med. 366(26):2455-65; PMID: 22658128); MPDL3280A/RG7446, or MEDI4736. In another embodiment, the PD-1 pathway inhibitor is a protein comprising (or a nucleic acid coding for) an amino acid sequence capable of binding to PD-1 but preventing PD-1 pathway signaling. In one embodiment, the PD-1 pathway inhibitor is a fusion protein of a fragment of PD-L1 or PD-L2 ligand. In one embodiment, the PD-1 pathway inhibitor is a fusion protein comprising the extracellular domain of PD-L1 or PD-L2 or a fragment thereof capable of binding to PD-1 and an Fc portion of an immunoglobulin. An example of such a fusion protein is represented by AMP-224 (extracellular domain of murine PD-L2/B7-DC fused to the unmodified Fc portion of murine IgG2a protein; Mkrtichyan et al., 2012. J Immunol. 189(5):2338-47; PMID: 22837483).

### Histamine

In one embodiment, histamine is used in the methods and compositions of the present invention. In another embodiment, a histamine receptor agonist is used in the methods and compositions of the present invention. In one embodiment, the histamine of the present invention is histamine dihydrochloride. In another embodiment, the histamine of the present invention is N-methyl-histamine. In another embodiment, the histamine of the present invention is 4-methyl-histamine. In another embodiment, the histamine of the present invention comprises other histamine H2-receptor agonists.

Histamine dihydrochloride is commercially available and methods of making histamine dihydrochloride as well as other forms of histamine are known in the art (for e.g. U.S. Pat. No. 6,528,654, which is incorporated herein by reference).

### Modes of Administration

In one embodiment, the administration of the PD-1 or PD-L1 inhibitors and the histamine may occur either simultaneously or time-staggered, either at the same site of administration or at different sites of administration.

In another embodiment, any of the therapeutic or prophylactic drugs or compounds described herein may be administered simultaneously. In another embodiment, they may be administered at different time than one another. In one embodiment, they may be administered within a few minutes of one another. In another embodiment, they may be administered within a few hours of one another. In another embodiment, they may be administered within 1 hour of one another. In another embodiment, they may be administered within 2 hours of one another. In another embodiment, they may be administered within 5 hours of one another. In another embodiment, they may be administered within 12 of one another. In another embodiment, they may be administered within 24 hours of one another.

In one embodiment, any of the therapeutic or prophylactic drugs or compounds described herein may be administered at the same site of administration. In another embodiment, they may be administered at different sites of administration.

### Immunostimulants

In another embodiment, the methods of the present invention in which PD-1 or PD-L1 inhibitors and histamine are administered further comprise the step of administering an additional immunostimulant. In one embodiment, the additional immunostimulant is interleukin-2 (IL-2). The administration of IL-2 may occur either simultaneously or time-staggered, either at the same site of administration or at different sites of administration as the PD-1 or PD-L1 inhibitors and the histamine.

In another embodiment, a PD-1 or PD-L1 inhibitors and histamine are administered as a combination treatment with an inhibitor of LAG-3, an inhibitor of indolamine 2,3-dioxygenase 1 (IDO1), sipuleucel-T, or other vaccinations, or a combination thereof. In another embodiment, PD-1 or PD-L1 inhibitors and histamine are administered as a combination treatment with anti-CD40, anti-CD27, anti-4-1BB, or a combination thereof. In another embodiment, PD-1 or PD-L1 inhibitors and histamine are administered as a combination treatment with other immunostimulants. In one embodiment, the immunostimulant is IFN, IL-21, anti-killer immunoglobulin-like receptor [KIR], or a combination thereof.

In another embodiment, a cancer vaccine is administered in the methods of the present invention in addition to the administration of PD-1 or PD-L1 inhibitors and histamine. In one embodiment, the vaccine is Gardasil®. In another embodiment, the vaccine is Cervarix®. In another embodiment, the vaccine is sipuleucel-T.

In another embodiment, the present invention provides a kit for prolonging remission from a cancer in a subject comprising a) a therapeutic amount of histamine dihydrochloride and PD-1 or PD-L1 inhibitors, and instructions for the use of said kit.

In one embodiment, histamine is an immunostimulant. In one embodiment, the kit comprises an additional immunostimulant in addition to histamine dihydrochloride. In one embodiment, the additional immunostimulant is IL-2. In another embodiment, the kit comprises an additional treatment, including the ones described hereinabove, in addition to histamine dihydrochloride and PD-1 or PD-L1 inhibitors.

### Predicting the Efficacy of a Cancer Treatment

In another embodiment, the present invention provides a method of predicting the efficacy of a cancer treatment in a subject.

In one embodiment, a biomarker is used to predict the efficacy of a cancer treatment in a subject. In one embodiment, the biomarker is H2R. In another embodiment, the biomarker is NKp30. In another embodiment, the biomarker is NKp46.

In another embodiment, the biomarker is Human Leukocyte Antigen—antigen D Related (HLA-DR), CD86, CD40, or a combination thereof. In another embodiment, the biomarker is low expression of HLA-ABC. In another embodiment, a reduction of blood monocyte counts prognosticates leukemia-free survival.

In another embodiment, the present invention provides a method of predicting the efficacy of a cancer treatment in a subject comprising the steps of: (a) obtaining a first blood sample from said subject; (b) administering a cycle of a treatment or a proposed treatment for said cancer to said subject; (c) obtaining a second blood sample from said subject after completion of said cycle of treatment; and (d) measuring frequency of NK cells in said first blood sample and said second blood sample, wherein if there is an increase in the frequency of NK cells in said second blood sample compared to said first blood sample, then said cancer treatment is predicted to be effective in said subject and wherein if there is no increase in the frequency of NK cells in said second blood sample compared to said first blood sample, then said cancer treatment is predicted not to be effective in said subject, thereby predicting the efficacy of said cancer treatment in said subject.

In another embodiment, the present invention provides a method of predicting the efficacy of a cancer treatment in a subject comprising the steps of: (a) obtaining a first blood sample from said subject; (b) administering a cycle of a treatment or a proposed treatment for said cancer to said subject; (c) obtaining a second blood sample from said subject after completion of said cycle of treatment; and (d) measuring the levels of a biomarker expression in said first blood sample and said second blood sample, wherein if there is an increase in the biomarker expression level in said second blood sample compared to said first blood sample, then said cancer treatment is predicted to be effective in said subject and wherein if there is no increase in the biomarker expression level in said second blood sample compared to said first blood sample, then said cancer treatment is predicted to be ineffective in said subject, thereby predicting the efficacy of said cancer treatment in said subject.

In another embodiment, the present invention provides a method of predicting the efficacy of a cancer treatment in a subject comprising the steps of: (a) obtaining a first blood sample from said subject; (b) administering a cycle of a treatment or a proposed treatment for said cancer to said subject; (c) obtaining a second blood sample from said subject after completion of said cycle of treatment; and (d) measuring the levels of a biomarker expression in said first blood sample and said second blood sample, wherein if there is an decrease in the biomarker expression level in said second blood sample compared to said first blood sample, then said cancer treatment is predicted to be effective in said subject and wherein if there is no decrease in the biomarker expression level in said second blood sample compared to said first blood sample, then said cancer treatment is predicted not to be effective in said subject, thereby predicting the efficacy of said cancer treatment in said subject.

In one embodiment, methods of predicting the efficacy of a cancer treatment as described herein may be used in conjunction with a method of preventing relapse to a cancer in a subject in remission from said cancer, a method of prolonging remission from a cancer in a subject, a method of preventing relapse to acute myeloid leukemia (AML) in a subject with AML in complete remission (CR) after chemotherapy, or a combination thereof, wherein said method comprises the steps of: a) obtaining a first blood sample from said subject; b) administering a cycle of a treatment or a proposed treatment for said cancer to said subject; c) obtaining a second blood sample from said subject after completion of said cycle of treatment; d) measuring the biomarker used in the method of predicting the efficacy of the cancer treatment in the subject in said first blood sample and said second blood sample, and e) administering additional cycles of said treatment to said subject if said biomarker has changed from said first blood sample to said second blood sample in a way that is predictive of the efficacy of the cancer treatment as described herein.

### Re-Distribution of Cytotoxic T Cells

In one embodiment, the present invention provides a method of predicting the efficacy of a cancer treatment in a subject comprising the steps of: a) obtaining a first blood sample from said subject; b) administering a cycle of a treatment or a proposed treatment for said cancer to said subject; c) obtaining a second blood sample from said subject after completion of said cycle of treatment; and d) measuring the frequency of CD8+ cytotoxic T cell phenotypes in said first blood sample and said second blood sample, wherein if there is a re-distribution of cytotoxic T cells such that there is a reduction in the frequency of T effector memory cells (T_{EM}) and an increase in the frequency of T effector cells (T_{eff}) in said second blood sample compared to said first blood sample, then said cancer treatment is predicted to be effective in said subject and wherein if there is no re-distribution of cytotoxic T cells in said second blood sample compared to said first blood sample, then said cancer treatment is predicted not to be effective in said subject, thereby predicting the efficacy of said cancer treatment in said subject.

In another embodiment, the present invention provides a method of preventing relapse to a cancer in a subject in remission from said cancer comprising the steps of: a) obtaining a first blood sample from said subject; b) administering a cycle of a treatment or a proposed treatment for said cancer to said subject; c) obtaining a second blood sample from said subject after completion of said cycle of treatment; d) measuring the frequency of CD8+ cytotoxic T cell phenotypes in said first blood sample and said second blood sample, and e) administering additional cycles of said treatment to said subject if there is a re-distribution of cytotoxic T cells such that there is a reduction in the frequency of T effector memory cells (T_{EM}) and an increase in the frequency of T effector cells (T_{eff}) in said second blood sample compared to said first blood sample, thereby preventing relapse to said cancer in said subject.

In another embodiment, the present invention provides a method of prolonging remission from a cancer in a subject comprising the steps of: a) obtaining a first blood sample from said subject; b) administering a cycle of a treatment or a proposed treatment for said cancer to said subject; c) obtaining a second blood sample from said subject after completion of said cycle of treatment; d) measuring the frequency of CD8+ cytotoxic T cell phenotypes in said first blood sample and said second blood sample, and e) administering additional cycles of said treatment to said subject if there is a re-distribution of cytotoxic T cells such that there is a reduction in the frequency of T effector memory cells (T_{EM}) and an increase in the frequency of T effector cells (T_{eff}) in said second blood sample compared to said first blood sample, thereby prolonging remission from said cancer in said subject.

In another embodiment, the present invention provides a method of preventing relapse to acute myeloid leukemia (AML) in a subject with AML in complete remission (CR) comprising the steps of: a) obtaining a first blood sample from said subject; b) administering a first cycle of histamine dihydrochloride and interleukin-2 (IL-2) to said subject; c) obtaining a second blood sample from said subject after completion of said first cycle of treatment; d) measuring the frequency of CD8+ cytotoxic T cell phenotypes in said blood sample; and e) administering additional cycles of histamine dihydrochloride and IL-2 to said subject if there is a re-distribution of cytotoxic T cells in said second blood sample compared to said first blood sample such that there is a reduction in the frequency of T effector memory cells (T_{EM}) and an increase in the frequency of T effector cells (T_{eff}), thereby preventing relapse to AML in said subject.

### Reactive Oxygen Species (ROS)

In another embodiment, the present invention provides a method of treating a cancer in a subject comprising the step of: administering a therapeutic amount of an agent that decreases reactive oxygen species (ROS). In one embodiment, the method comprises the step of administering a therapeutic amount of an agent that decreases extracellular ROS.

In one embodiment, the agent is an inhibitor of ROS formation. In another embodiment, the agent is a scavenger of extracellular ROS.

In one embodiment, the method further comprises the step of administering a histamine receptor agonist. In one embodiment, the cancer is leukemia. In one embodiment, the leukemia is chronic myelomonocytic leukemia (CMML).

### Cancers

In one embodiment, methods of treating cancer as described herein comprise methods of preventing or delaying relapse to a cancer in a subject in remission, prolonging remission from cancer, increasing survival, decreasing or alleviating cancer symptoms, or a combination thereof. In another embodiment, methods of treating cancer as described herein comprise methods of reducing the tumor burden in a subject with primary or metastatic cancer in a subject, reducing the risk of metastatic tumor spread in a subject with active cancer, preventing or delaying the reappearance, recurrence or metastatic spread of cancer in a subject, preventing relapse to a cancer in a subject, delaying the relapse to a cancer in a subject in remission from said cancer, prolonging the remission from a cancer in a subject, increasing the survival of a subject in remission from a cancer, prolonging the survival time of a subject in remission from a cancer, reducing malignant tumor growth in a subject, decreasing or alleviating cancer symptoms in a subject in remission from a cancer, or a combination thereof.

In one embodiment, a subject treated by the methods of the present invention has or is suffering from a cancer or tumor. In another embodiment, a subject treated by the methods of the present invention is in remission from a cancer or tumor. In another embodiment, the subject is in complete remission (CR). In one embodiment, the CR is from leukemia. Methods of evaluating complete remission are well known in the art.

In another embodiment, a subject treated by the methods of the present invention has a solid tumor or lymphoma and showed a “response” to earlier cancer treatment. In another embodiment, a subject treated by the methods of the present invention has a solid tumor or lymphoma and showed a “complete response” to earlier cancer treatment.

In one embodiment, the subject having cancer or a tumor has been treated with surgery, chemotherapy, radiation therapy, a targeted therapy, including therapies that are intended to boost immune system responses against cancer, or a combination thereof. In another embodiment, the subject having cancer or a tumor is being treated with histamine and PD-1 or PD-L1 inhibitors.

In one embodiment, the subject in the methods of the present invention has or had cancer. In another embodiment, the subject has or had a tumor. In another embodiment, the subject has a neoplastic disease. In another embodiment, the subject has a malignancy. In another embodiment, the subject has or had a pre-cancerous condition or a pre-malignant condition.

In one embodiment, the cancer is a leukemia. In one embodiment, the leukemia is Acute myeloid leukemia (AML). In another embodiment, the leukemia is Chronic myeloid leukemia (CML). In another embodiment, the leukemia is Chronic myelomonocytic leukemia (CMML). In another embodiment, the leukemia is Acute lymphocytic leukemia (ALL). In another embodiment, the leukemia is Chronic lymphocytic leukemia (CLL). In another embodiment, the leukemia is hairy cell leukemia.

In one embodiment, the tumor is a solid tumor. In one embodiment, the solid tumor is a colon carcinoma, prostate cancer, breast cancer, lung cancer, skin cancer, liver cancer, bone cancer, ovary cancer, pancreas cancer, brain cancer, head and neck cancer or other solid tumor.

In one embodiment, the cancer or tumor is in the breast, prostate, lung, colon, stomach, pancreas, ovary, or brain. In another embodiment, the cancer is a hematopoietic cancer, a neuroblastoma, or a malignant glioma.

In another embodiment, the cancer is selected from one or more of the following: Adrenocortical Carcinoma, AIDS-Related Cancers, Kaposi Sarcoma, AIDS-Related Lymphoma, Primary CNS Lymphoma, Anal Cancer, Appendix Cancer, Astrocytomas, Atypical Teratoid/Rhabdoid Tumor, Central Nervous System, Basal Cell Carcinoma—see Skin Cancer (Nonmelanoma), Bile Duct Cancer, Bladder Cancer, Bone Cancer, Ewing Sarcoma Family of Tumors, Osteosarcoma and Malignant Fibrous Histiocytoma, Brain Stem Glioma, Brain Tumor, Astrocytomas, Brain and Spinal Cord Tumors Treatment Overview, Brain Stem Glioma, Central Nervous System Atypical Teratoid/Rhabdoid Tumor, Central Nervous System Embryonal Tumors, Central Nervous System Germ Cell Tumors, Craniopharyngioma, Ependymoma, Breast Cancer, Bronchial Tumors, Burkitt Lymphoma—see Non-Hodgkin Lymphoma, Carcinoid Tumor, Gastrointestinal, Carcinoma of Unknown Primary, Cardiac (Heart) Tumors, Central Nervous System, Atypical Teratoid/Rhabdoid Tumor, Embryonal Tumors, Germ Cell Tumor, Lymphoma, Primary, Cervical Cancer, Cholangiocarcinoma, Chordoma, Chronic Myeloproliferative Neoplasms, Colon Cancer, Colorectal Cancer, Craniopharyngioma, Cutaneous T-Cell Lymphoma—see Mycosis Fungoides and Sezary Syndrome, Ductal Carcinoma In Situ (DCIS), Embryonal Tumors, Central Nervous System, Endometrial Cancer, Ependymoma, Esophageal Cancer, Esthesioneuroblastoma, Ewing Sarcoma, Extracranial Germ Cell Tumor, Extragonadal Germ Cell Tumor, Eye Cancer, Intraocular Melanoma, Retinoblastoma, Fallopian Tube Cancer, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors (GIST), Germ Cell Tumor, Central Nervous System, Extracranial, Extragonadal, Ovarian, Testicular, Gestational Trophoblastic Disease, Glioma—see Brain Tumor Brain Stem, Head and Neck Cancer, Heart Cancer, Hepatocellular (Liver) Cancer, Histiocytosis, Langerhans Cell, Hodgkin Lymphoma, Hypopharyngeal Cancer, Intraocular Melanoma, Islet Cell Tumors, Pancreatic Neuroendocrine Tumors, Kaposi Sarcoma, Kidney, Renal Cell, Wilms Tumor and Other Childhood Kidney Tumors, Langerhans Cell Histiocytosis, Laryngeal Cancer, Acute Lymphoblastic (ALL), Acute Myeloid (AML), Chronic Lymphocytic (CLL), Chronic Myelogenous (CML), Hairy Cell, Lip and Oral Cavity Cancer, Liver Cancer (Primary), Lung Cancer, Non-Small Cell, Small Cell, Lymphoma, AIDS-Related, Burkitt—see Non-Hodgkin Lymphoma, Cutaneous T-Cell—see Mycosis Fungoides and Sezary Syndrome, Hodgkin, Non-Hodgkin, Primary Central Nervous System (CNS), Macroglobulinemia, Waldenström—see Non-Hodgkin Lymphoma, Male Breast Cancer, Malignant Fibrous Histiocytoma of Bone and Osteosarcoma, Melanoma, Intraocular (Eye), Merkel Cell Carcinoma, Mesothelioma, Malignant, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myeloma, Multiple, Myeloproliferative Neoplasms, Chronic, Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip and, Oropharyngeal Cancer, Osteosarcoma and Malignant Fibrous Histiocytoma of Bone, Ovarian Cancer, Epithelial, Germ Cell Tumor, Low Malignant Potential Tumor, Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Pregnancy and Breast Cancer, Primary Central Nervous System (CNS) Lymphoma, Primary Peritoneal Cancer, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma, Ewing, Kaposi, Osteosarcoma (Bone Cancer), Rhabdomyosarcoma, Soft Tissue, Uterine, Vascular Tumors, Sezary Syndrome, Skin Cancer, Melanoma, Merkel Cell Carcinoma, Nonmelanoma, Small Cell Lung Cancer, Small Intestine Cancer, Soft Tissue Sarcoma, Squamous Cell Carcinoma—see Skin Cancer (Nonmelanoma), Squamous Neck Cancer with Occult Primary, Metastatic, Stomach (Gastric) Cancer, T-Cell Lymphoma, Cutaneous—see Mycosis Fungoides and Sezary Syndrome, Testicular Cancer, Throat Cancer, Thymoma and Thymic Carcinoma, Thyroid Cancer, Transitional Cell Cancer of the Renal Pelvis and Ureter, Primary Carcinoma, Ureter and Renal Pelvis, Transitional Cell Cancer, Urethral Cancer, Uterine Cancer, Endometrial, Uterine Sarcoma, Vaginal Cancer, Vascular Tumors, Vulvar Cancer, Waldenström Macroglobulinemia—see Non-Hodgkin Lymphoma, and Wilms Tumor.

In one embodiment, the pre-cancerous condition is actinic keratosis, Barrett's esophagus, atrophic gastritis, ductal carcinoma in situ, dyskeratosis congenita, sideropenic dysphagia, lichen planus, oral submucous fibrosis, solar elastosis, cervical dysplasia, leukoplakia, erythroplakia, or a combination thereof. In one embodiment, the pre-cancerous condition is a dysplasia or a benign neoplasia. In one embodiment, the pre-cancerous condition is prostatic intraepithelial neoplasia (PIN), proliferative inflammatory atrophy (PIA), atypical small acinar proliferation (ASAP), squamous intraepithelial lesion (SIL), Atypical endometrial hyperplasia, ovarian epithelial dysplasia, Breast calcifications, MGUS (mono gammopathy of unknown significance), Vulval intra-epithelial neoplasia (VIN), Lobular carcinoma in situ (LCIS) Vaginal intra-epithelial neoplasia, or VAIN Vulval lichen sclerosus and lichen planus, Cervical intra-epithelial neoplasia (CIN), Barrett's oesophagus, or a combination thereof.

In one embodiment, the cancer treatment described in the methods of the present invention is given to a subject in remission. In one embodiment, the subject is in remission from a hematopoietic cancer. In one embodiment, it is given to a subject who has undergone induction therapy. In one embodiment, induction chemotherapy comprises a combination of cytarabine and daunorubicin. In one embodiment, it is given to a subject who has completed consolidation therapy. In one embodiment, consolidation chemotherapy comprises a combination of cytarabine and daunorubicin. In another embodiment, the consolidation phase comprises 2-4 courses of high-dose cytarabine, sometimes with the addition of an anthracyline (in one embodiment, daunorubicin or, in another embodiment, idarubicin). In another embodiment, the consolidation phase comprises high-dose cytarabine without the addition of anthracyclines. In another embodiment, the consolidation phase comprises an allogeneic or autologous transplant. In another embodiment, the subject receives an allogeneic or autologous transplant after the completion of the consolidation phase.

In one embodiment, the cancer treatment is an immunological treatment or an immunotherapy. In one embodiment, the treatment strengthens the immune response in the subject to the cancer. In another embodiment, the treatment induces an immune response in the subject to the cancer. In one embodiment, the treatment comprises administration of a cytokine to the subject. In one embodiment, the cytokine is an immunostimulant.

### Cytokines

In one embodiment, the cytokine is an interleukin. In one embodiment, the interleukin is IL-2. In another embodiment, the interleukin is IL-12. In another embodiment, the interleukin is IL-15. In one embodiment, the interleukin is administered at a low dose.

In one embodiment, IL-2 is administered at a dosage of 16,400 U/kg two times per day.

In another embodiment, the cytokine is an interferon. In one embodiment, the interferon is interferon-alpha. In another embodiment, the interferon is interferon-beta. In another embodiment, the interferon is interferon-gamma

In another embodiment, the cytokine is a hematopoietic growth factor. In one embodiment, the hematopoietic growth factor is selected from the group consisting of: Erythropoietin, IL-11, Granulocyte-macrophage colony-stimulating factor (GM-CSF), and granulocyte colony-stimulating factor (G-CSF), or a combination thereof.

In one embodiment, methods of the present invention include administering anti-CTLA-4 and compositions of the present invention comprise anti-CTLA-4. In one embodiment, CTLA4 or CTLA-4 (cytotoxic T-lymphocyte-associated protein 4), also known as CD152 (cluster of differentiation 152), is a protein receptor that, functioning as an immune checkpoint, downregulates the immune system. In one embodiment, anti-CTLA-4 is administered to a subject to improve the efficacy of the cancer treatment.

In one embodiment, the consolidation therapy given to a subject in remission or to a subject having cancer comprises administration of a histamine receptor agonist to the subject. In one embodiment, the histamine is an immunostimulant. In one embodiment, the histamine is histamine dihydrochloride.

In one embodiment, 0.5 mg of histamine dihydrochloride is administered.

In one embodiment, therapeutic agents as described herein, including IL-2, histamine, and PD-1 or PD-L1 inhibitors are administered once a day. In another embodiment, therapeutic agents are administered to a subject two times per day. In another embodiment, therapeutic agents are administered to a subject three times per day. In another embodiment, therapeutic agents are administered to a subject four times per day. In another embodiment, therapeutic agents are administered to a subject once a week. In another embodiment, therapeutic agents are administered to a subject two times per week. In another embodiment, therapeutic agents are administered to a subject three times per week. In another embodiment, therapeutic agents are administered to a subject four times per week. In another embodiment, therapeutic agents are administered to a subject once every two weeks.

In one embodiment, the cancer treatment comprises administering histamine dihydrochloride and interleukin-2 (IL-2). In one embodiment, histamine dihydrochloride administered at 0.5 mg two times per day and IL-2 administered at 16,400 U two times per day, as described in Example 3 hereinbelow, was highly effective at preventing relapse to patients in remission from AML, as described in Example 4 hereinbelow when the first or third round of treatments with IL-2 and histamine induced a re-distribution of cytotoxic T cells in which there was a reduction in the frequency of T effector memory cells and a concomitant increase of T effector cells.

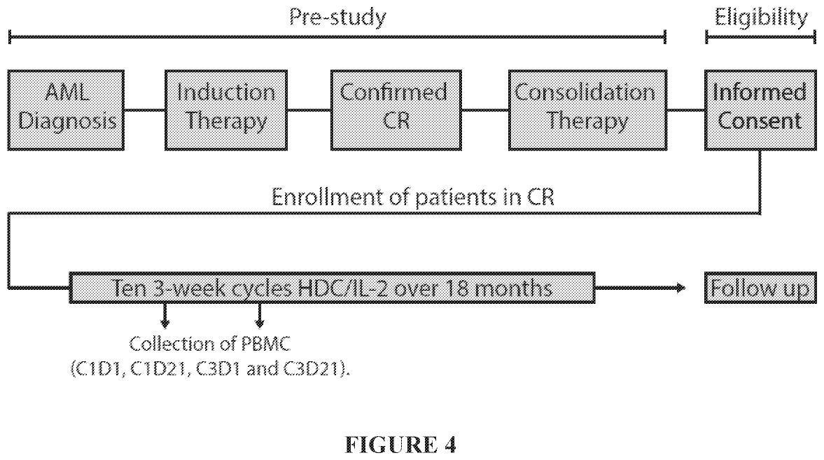

In one embodiment, the cancer treatment described in the methods herein is post-consolidation therapy, as described in FIG. 4, or maintenance therapy.

In one embodiment, some of the methods of the present invention are practiced on a first and second sample drawn from a subject before and after a round of post-consolidation therapy. In one embodiment, the samples are taken before and after the completion of post-consolidation therapy. In another embodiment, the samples are taken before and after the first round of post-consolidation therapy. In another embodiment, the samples are taken before and after the third round of post-consolidation therapy. In another embodiment, the samples are taken before and after the first and third rounds of post-consolidation therapy. In another embodiment, the samples are taken before and after the first through fourth round of post-consolidation therapy. In another embodiment, the samples are taken before and after the second round of post-consolidation therapy. In another embodiment, the samples are taken before and after the fourth round of post-consolidation therapy. In another embodiment, the samples are taken before the first round and after the third round of post-consolidation therapy. In another embodiment, the samples are taken before the first round and after the second round of post-consolidation therapy. In another embodiment, the samples are taken before the first round and after the fourth round of post-consolidation therapy.

In one embodiment, each treatment cycle lasts 3 weeks as described in Example 3. In another embodiment, each treatment cycle lasts 1-5 weeks. In another embodiment, each treatment cycle lasts 1 week. In another embodiment, each treatment cycle lasts 2 weeks. In another embodiment, each treatment cycle lasts 4 weeks.

In one embodiment, post-the consolidation therapy comprises 10 cycles, as described in Example 3 hereinbelow. In another embodiment, the consolidation therapy comprises 1-5 cycles. In another embodiment, the consolidation therapy comprises 5-10 cycles. In another embodiment, the consolidation therapy comprises 10-15 cycles. In another embodiment, the consolidation therapy comprises 5 cycles. In another embodiment, the consolidation therapy comprises 15 cycles. In another embodiment, the consolidation therapy comprises 7 cycles. In another embodiment, the consolidation therapy comprises 12 cycles. In another embodiment, the consolidation therapy comprises 3 cycles.

In one embodiment, the samples from the subject are tissue samples. In another embodiment, the samples from the subject are blood samples. In one embodiment, the blood sample is a peripheral blood sample. In another embodiment, the samples from the subject are cerebrospinal fluid (CSF) samples. In another embodiment, the samples from the subject are urine samples. In another embodiment, the samples from the subject are fecal samples.

### Kits

In another embodiment, the present invention provides a kit for predicting the efficacy of a cancer treatment in a subject comprising a therapeutic amount of histamine dihydrochloride, an immunostimulant, a means for measuring CD8+ cytotoxic phenotypes, and instructions for the use of said kit.

In another embodiment, the present invention provides a kit for preventing relapse to a cancer in a subject in remission from said cancer comprising a therapeutic amount of histamine dihydrochloride, an immunostimulant, a means for measuring CD8+ cytotoxic phenotypes, and instructions for the use of said kit.

In another embodiment, the present invention provides a kit for prolonging remission from a cancer in a subject comprising a therapeutic amount of histamine dihydrochloride, an immunostimulant, a means for measuring CD8+ cytotoxic phenotypes, and instructions for the use of said kit.

In another embodiment, the present invention provides a kit for preventing relapse to acute myeloid leukemia (AML) in a subject with AML in complete remission (CR) after chemotherapy comprising a therapeutic amount of histamine dihydrochloride, an immunostimulant, a means for measuring CD8+ cytotoxic phenotypes, and instructions for the use of said kit.

In another embodiment, the present invention provides kits for predicting the efficacy of a cancer treatment in a subject comprising a therapeutic amount of histamine dihydrochloride, an immunostimulant, a means for measuring a biomarker as described herein, and instructions for the use of said kit.

In another embodiment, the present invention provides a kit for preventing relapse to a cancer in a subject in remission from said cancer comprising a therapeutic amount of histamine dihydrochloride, an immunostimulant, a means for measuring a biomarker as described herein, and instructions for the use of said kit.

In another embodiment, the present invention provides a kit for prolonging remission from a cancer in a subject comprising a therapeutic amount of histamine dihydrochloride, an immunostimulant, a means for measuring a biomarker as described herein, and instructions for the use of said kit.

In another embodiment, the present invention provides a kit for preventing relapse to acute myeloid leukemia (AML) in a subject with AML in complete remission (CR) after chemotherapy comprising a therapeutic amount of histamine dihydrochloride, an immunostimulant, a means for measuring a biomarker as described herein, and instructions for the use of said kit.

In one embodiment, a cancer treatment of the present invention prevents relapse to a cancer. In another embodiment, a cancer treatment of the present invention prevents recurrence of a cancer. In another embodiment, a cancer treatment of the present invention delays the relapse of a cancer. In another embodiment, a cancer treatment of the present invention delays the recurrence of a cancer. In another embodiment, a cancer treatment of the present invention prolongs remission from a cancer. In another embodiment, a cancer treatment of the present invention increases the survival of a subject with cancer. In another embodiment, a cancer treatment of the present invention prolongs the survival time of a subject with cancer. In another embodiment, a cancer treatment of the present invention treats cancer in a subject. In another embodiment, a cancer treatment of the present invention decreases cancer symptoms in a subject. In another embodiment, a cancer treatment of the present invention alleviates cancer symptoms in a subject. In another embodiment, a cancer treatment of the present invention prolongs cancer-free remission. In another embodiment, a cancer treatment of the present invention reduces the size of malignant tumors in a subject. In another embodiment, a cancer treatment of the present invention reduces the tumor burden in patients with primary or metastatic cancer. In another embodiment, a cancer treatment of the present invention reduces the risk of metastatic tumor spread in patients with active cancer. In another embodiment, a cancer treatment of the present invention prevents or delays the reappearance, recurrence or metastatic spread of cancer in patients who have undergone surgery, chemotherapy or any other treatment to reduce the cancer burden.

In one embodiment, remission comprises remission from hematopoietic cancer. In one embodiment, remission from hematopoietic cancer is when subjects are microscopically free of cancer cells (in bone marrow, blood or other organs) along with the re-appearance of normal hematopoiesis, in one embodiment, after induction chemotherapy.

In one embodiment, cancer remission is a decrease in or disappearance of signs and symptoms of cancer. In one embodiment, the remission is a partial remission, which in one embodiment, is when some, but not all, signs and symptoms of cancer have disappeared. In another embodiment, the remission is a complete remission, which in one embodiment, is when all signs and symptoms of cancer have disappeared, although cancer still may be in the body.

In one embodiment, a subject goes into remission after induction therapy, which in one embodiment, comprises surgery, chemotherapy or other means of reducing a tumor burden.

In one embodiment, “treating” refers to both therapeutic treatment and prophylactic or preventative measures, wherein the object is to prevent or lessen the targeted pathologic condition or disorder as described hereinabove. Therefore, in one embodiment, methods of the present invention comprise administering to a subject while the subject is in remission from cancer after chemotherapy or surgery or other treatments. In another embodiment, methods of the present invention comprise administering to a subject who has relapsed to cancer. In another embodiment, methods of the present invention comprise administering to a subject who has active cancer.

Thus, in one embodiment, treating may include directly affecting or curing, suppressing, inhibiting, preventing, reducing an incidence, reducing the severity of, delaying the onset of, reducing symptoms associated with the disease, disorder or condition, or a combination thereof. Thus, in one embodiment, “treating” refers inter alia to delaying progression, expediting remission, inducing remission, augmenting remission, speeding recovery, increasing efficacy of or decreasing resistance to alternative therapeutics, or a combination thereof. In another embodiment, treating refers to reducing the pathogenesis of, ameliorating the symptoms of, ameliorating the secondary symptoms of, or prolonging the latency to a relapse of a cancer in a subject. In one embodiment, “preventing” refers, inter alia, to delaying the onset of symptoms, preventing relapse to a disease, decreasing the number or frequency of relapse episodes, increasing latency between symptomatic episodes, or a combination thereof. In one embodiment, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

In one embodiment, methods of the present invention alleviate symptoms in a subject. In one embodiment, symptoms are primary, while in another embodiment, symptoms are secondary. In one embodiment, “primary” refers to a symptom that is a direct result of the cancer, while in one embodiment, “secondary” refers to a symptom that is derived from or consequent to a primary cause. In one embodiment, the methods of the present invention treat primary or secondary symptoms or secondary complications related to cancer.

In another embodiment, “symptoms” may be any manifestation of a cancer, including twitching, cramping, stiffness of muscles; muscle weakness affecting an arm or a leg; slurred or nasal speech; difficulty chewing or swallowing; general weakness, atrophy, or a combination thereof.

According to any of the methods of the present invention and in one embodiment, the subject is human. In another embodiment, the subject is a non-human primate. In another embodiment, the subject is murine, which in one embodiment is a mouse, and, in another embodiment is a rat. In another embodiment, the subject is canine, feline, bovine, equine, laprine or porcine. In another embodiment, the subject is mammalian. In another embodiment, the subject is any organism susceptible to cancer or tumors.

### Therapeutic Compositions and Methods of Administration

In one embodiment, the methods of the present invention comprise administering a pharmaceutical composition comprising a therapeutic agent as described herein, including inhibitors of PD-1 or PD-L1, histamine dihydrochloride, and IL-2, and a pharmaceutically acceptable carrier.

“Pharmaceutical composition” refers, in one embodiment, to a therapeutically effective amount of the active ingredient, i.e. inhibitors of PD-1 or PD-L1, histamine dihydrochloride, or IL-2, together with a pharmaceutically acceptable carrier or diluent. A “therapeutically effective amount” refers, in one embodiment, to that amount which provides a therapeutic effect for a given condition and administration regimen.

The pharmaceutical compositions containing the therapeutic agent can be, in one embodiment, administered to a subject by any method known to a person skilled in the art, such as parenterally, transmucosally, transdermally, intramuscularly, intravenously, intra-dermally, intra-peritonealy, intra-ventricularly, intra-cranially, intra-vaginally or intra-tumorally. In one embodiment, the therapeutic agent is administered subcutaneously.

In another embodiment as described in the methods and compositions of the present invention, the pharmaceutical compositions are administered orally, and are thus formulated in a form suitable for oral administration, i.e. as a solid or a liquid preparation. Suitable solid oral formulations include tablets, capsules, pills, granules, pellets and the like. Suitable liquid oral formulations include solutions, suspensions, dispersions, emulsions, oils and the like. In another embodiment of the present invention, the active ingredient is formulated in a capsule. In accordance with this embodiment, the compositions of the present invention comprise, in addition to the active compound and the inert carrier or diluent, a hard gelating capsule.

In another embodiment, the pharmaceutical compositions are administered by intravenous, intra-arterial, or intra-muscular injection of a liquid preparation. Suitable liquid formulations include solutions, suspensions, dispersions, emulsions, oils and the like. In another embodiment, the pharmaceutical compositions are administered intravenously and are thus formulated in a form suitable for intravenous administration. In another embodiment, the pharmaceutical compositions are administered intra-arterially and are thus formulated in a form suitable for intra-arterial administration. In another embodiment, the pharmaceutical compositions are administered intra-muscularly and are thus formulated in a form suitable for intra-muscular administration.

In another embodiment, the pharmaceutical compositions are administered topically to body surfaces and are thus formulated in a form suitable for topical administration. Suitable topical formulations include gels, ointments, creams, lotions, drops and the like. For topical administration, the therapeutic agent is prepared and applied as a solution, suspension, or emulsion in a physiologically acceptable diluent with or without a pharmaceutical carrier.

In another embodiment, the pharmaceutical compositions provided herein are controlled-release compositions, i.e. compositions in which the therapeutic agent is released over a period of time after administration. Controlled- or sustained-release compositions include formulation in lipophilic depots (e.g. fatty acids, waxes, oils). In another embodiment, the composition is an immediate-release composition, i.e. a composition in which all of the therapeutic agent is released immediately after administration.

Each of the above additives, excipients, formulations and methods of administration represents a separate embodiment of the present invention.

All patents, patent applications, and scientific publications cited herein are hereby incorporated by reference in their entirety.

The following examples are presented in order to more fully illustrate the preferred embodiments of the invention. They should in no way be construed, however, as limiting the broad scope of the invention.

### EXAMPLES

### Example 1

### Combination of Anti-PD-1/Anti-PDL1 and Histamine—Methods

The EL-4 thymoma cell line (provided by Dr. Ingo Schmitz, Otto-von-Guericke-University, Magdeburg, Germany) was cultured in DMEM medium (Sigma, Stockholm, Sweden) supplemented with 10% fetal calf serum, 100 U/ml penicillin, 100 μg/ml streptomycin and 2 mM L-glutamine, as described in Martner, A. et al. The Journal of Immunology, volume 194, no. 10, pp. 5014-5021. C57BL/6J mice (6-8 weeks old, purchased from Charles River Laboratories, Sulzfeld, Germany) were inoculated subcutaneously with 1.75-2×10⁵ EL-4 tumor cells and treated with saline (control) or 1.5 mg histamine dihydrochloride (purchased from Sigma-Aldrich, Stockholm, Sweden) diluted in saline, by intraperitoneal injections three times per week starting one day before inoculation of cells. Two-hundred forty μg of an antibody against the programmed cell death-1 receptor (anti-mouse PD1, clone RMP1-14, referred to as a-PD1) and 240 μg of an antibody against the programmed death-ligand 1 (anti-mouse PD-L1, clone 10F.9G2, referred to as α-PDL1) (both purchased from BioXcell, West Lebanon, N.H., USA, catalogue numbers BE0146 and BE0101, respectively) were administrated by intraperitoneal injections three, six and ten days after tumor inoculation. Tumors were measured manually every 2-3 days. Tumor size was calculated as length× width. Mice were sacrificed two weeks after tumor inoculation when control tumors had reached a size of approximately 1.5 cm².

### Example 2

### Combination of Anti-PD-1/Anti-PDL1 and Histamine—Results

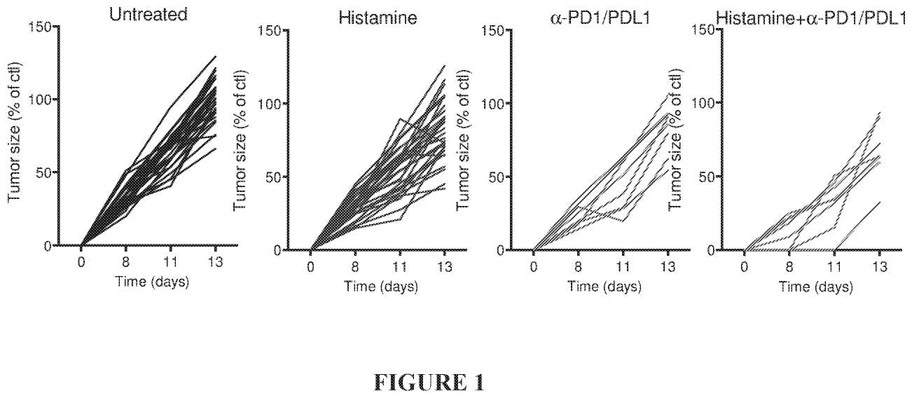

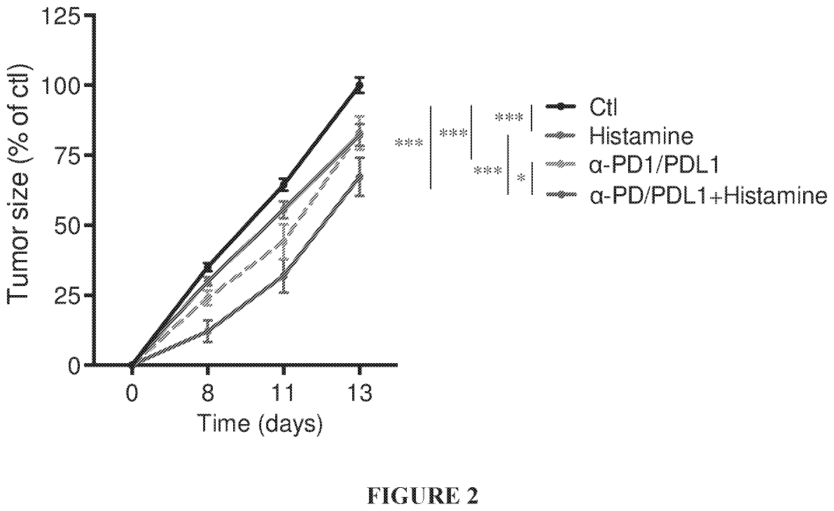

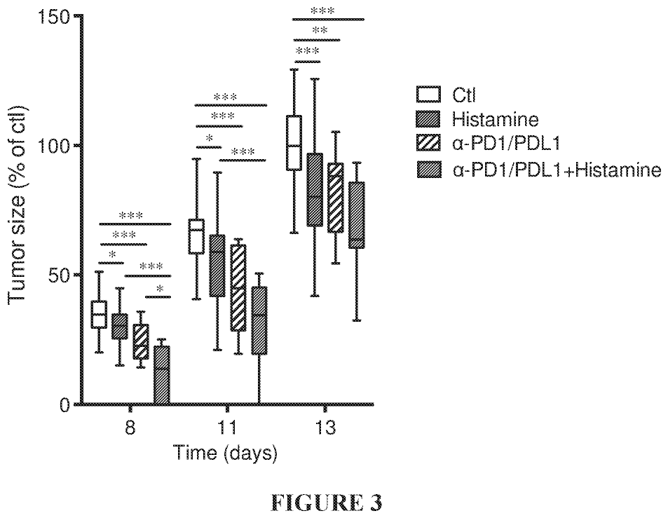

In agreement with a previous study (Martner, A. et al. The Journal of Immunology, volume 194, no. 10, pp. 5014-5021), treatment of EL-4-bearing mice with histamine significantly reduced tumor growth rate (FIGS. 1-3). The administration of α-PD1-+α-PDL1-inhibitory antibodies resulted in a comparable reduction of tumor growth. The combination of histamine and anti-PD1/anti-PDL1 increased the reduction of EL-4 tumor growth over each treatment (histamine or α-PD1/α-PDL1) alone. In FIGS. 1-3, tumor size is indicated as % of control, where 100% was the mean size of control tumors at the end of the experiments (i.e. at 2 weeks after tumor inoculation).

### Example 3

### Dynamics of Cytotoxic T Cell Subsets During Immunotherapy Predicts Outcome in Acute Myeloid Leukemia

### Materials and Methods

### Patients, Study Design and Objectives

This single-armed multicenter phase IV study (Re:Mission, NCT01347996, registered at www.clinicaltrials.gov) enrolled 84 patients (age 18-79) with AML in first CR. As outlined schematically in FIG. 4, the patients received ten consecutive 21-day cycles of histamine dihydrochloride (HDC; Ceplene) in combination with low-dose IL-2 during 18 months or until relapse or death.

The treatment continued for a total of 18 months or until the patients relapsed, died, discontinued therapy because of adverse events, withdrew consent, or became lost to follow-up. Cycles 1 to 3 comprised 3 weeks of treatment and 3 weeks off treatment, and in cycles 4 to 10 the off-treatment periods were extended to 6 weeks. In each cycle, patients in the treatment arm received HDC (Maxim Pharmaceuticals, San Diego, Calif.) at 0.5 mg subcutaneous twice a day and human recombinant IL-2 (aldesleukin; 16 400 U/kg subcutaneous twice a day; Chiron Corporation, Emeryville, Calif.). After 18 months of treatment (HDC/IL-2 arm), all patients were followed for at least six additional months after the end of immunotherapy.

The dosage, route of administration, exclusion criteria etc. were identical to those described for a previous phase III trial [Blood. 2006; 108(1):88-96, incorporated herein by reference]. Primary endpoints in the Re:Mission study included assessment of the quantitative and qualitative pharmacodynamic effects of HDC/IL-2 by monitoring T and NK cell phenotypes before and after treatment cycles. The protocol stated that all data collected in support of these objectives were to be analyzed for the populations as a whole and by subgroups according to patient age at enrolment (<60 and >60 years). The herein reported aspects of T cell biology vs. clinical outcome (LFS and OS) were performed post-hoc. Patient characteristics are presented in Table 1.

TABLE 1
Patient characteristics

| | n(%) | | LFS, n (%) | |
|---|---|---|---|---|
| | All patients | Age >60 | All | |
| | (n = 84) | (n = 47) | patients | Age >60 |
| Sex | | | | |
| Female | 44 (52) | 23 (49) | 15/44 (34) | 6/23 (26) |
| Male | 40 (48) | 24 (51) | 20/40 (50) | 10/24 (42) |
| Risk group | | | | |
| Favorable risk | 34 (40) | 17 (36) | 18/34 (53) | 8/17 (47) |
| Intermediate I | 25 (30) | 10 (21) | 9/25 (36) | 2/10 (20) |
| Intermediate II | 13 (15) | 9 (19) | 6/13 (46) | 4/9 (44) |
| High risk | 7 (8) | 6 (13) | 1/7 (14) | 1/6 (17) |
| ND | 5 (6) | 5 (11) | 1/5 (20) | 1/5 (20) |
| Karyotype | | | | |
| Normal | 44 (52) | 23 (49) | 19/44 (43) | 9/23 (39) |
| Favorable | 14 (17) | 5 (11) | 8/14 (57) | 2/5 (40) |
| Unfavorable | 7 (8) | 6 (13) | 2/7 (29) | 2/6 (33) |
| Other | 15 (18) | 10 (21) | 5/15 (33) | 3/10 (30) |
| ND | 4 (5) | 3 (6) | 1/4 (25) | 0/3 (0) |
| Mutation status | | | | |
| NPM1 | n = 69 | n = 39 | | |
| | 25 (36) | 14 (36) | 12/25 (48) | 6/14 (43) |
| FLT3 | n = 72 | n = 37 | | |
| | 6 (8) | 3 (8) | 0/6 (0) | 0/3 (0) |
| CEBPA | n = 42 | n = 23 | | |
| | 3 (7) | 1 (4) | 1/3 (33) | 0/1 (0) |
| Induction | | | | |
| courses | | | | |
| 1 | 63 (75) | 33 (70) | 31/63 (49) | 14/33 (44) |
| >1 | 21 (25) | 14 (30) | 4/21 (19) | 2/14 (14) |
| Consolidation | | | | |
| courses | | | | |
| 0-2 | 41 (49) | 27 (57) | 15/41 (37) | 6/27 (22) |
| >2 | 43 (51) | 20 (43) | 20/43 (47) | 10/20 (50) |

Abbreviations:
LFS, leukemia-free survival,
ND, not done

A more detailed account for previous induction and consolidation therapy can be found elsewhere.

### Sampling of Peripheral Blood and Flow Cytometry

Peripheral blood was collected before and after the first and third treatment cycles, i.e. cycle 1, day 1 (C1D1) and cycle 1, day 21 (C1D21), cycle 3, day 1 (C3D1) and cycle 3, day 21 (C3D21). PBMC were isolated and cryopreserved at local sites and shipped on dry ice to the central laboratory (at the Sahlgrenska Cancer Center, University of Gothenburg, Sweden) for analysis by use of flow cytometry. For these analyses, the frozen PBMC samples were thawed quickly in Iscoves' medium supplemented with 10% FCS. Subsequently, cells were washed in Iscoves' medium and thereafter in PBS. Cells were first stained with LIVE/DEAD fixable yellow stain (Life technologies, Grand Island, N.Y., USA) by incubation for 30 min at 4° C. in PBS, followed by staining for surface markers for 30 min at 4° C. in PBS containing 0.5% BSA and 0.1% EDTA or in brilliant stain buffer (BD Biosciences, Stockholm, Sweden). The following anti-human monoclonal antibodies were used for phenotyping: CD3-FITC (HIT3a), CD4-APC-H7 (RPA-T4), CD4-Horizon V450 (RPA-T4), CD8-APC (RPA-T8), CD8-PerCP-Cy5.5 (RPA-T8/SK1), CD8-Qdot705 (3B5), CD16-Horizon V450 (3G8), CD25-Brilliant Violet 421 (M-A251), CD45RA-APC (HI100), CD45RO-PE (UCHL1), CD56-PerCP-eFluor710 (CMSSB), CD56-PE-Cy7 (NCAM16.2), CD69-PE-Cy7 (FN50), HLA-DR-FITC (L243) (all from BD Biosciences). CCR7-PE-Cy7 (G043H7) from Biolegend, San Diego, Calif., USA. CD3-Pacific Blue (S4.1), CD14-Qdot655 (TüK4) and streptavidin-Qdot605 (all from Life technologies). Intracellular staining of IFN-γ-PE-Cy7 (B27; BD Biosciences) was performed after surface staining and fixation and permeabilization using the FoxP3 fixation/permeabilization kit (eBioscience, San Diego, Calif., USA) according to the manufacturer's instructions.

Stained samples were analyzed on a 4-laser BD LSRFortessa SORP flow cytometer (405, 488, 532, and 640 nm; BD Biosciences). Data were analyzed using the FlowJo software, version 7.6.5 or later (TreeStar, Ashland, Oreg., USA) or the FACSDiva software, version 6 or later (BD Biosciences). Samples with less than 25% viability were excluded.

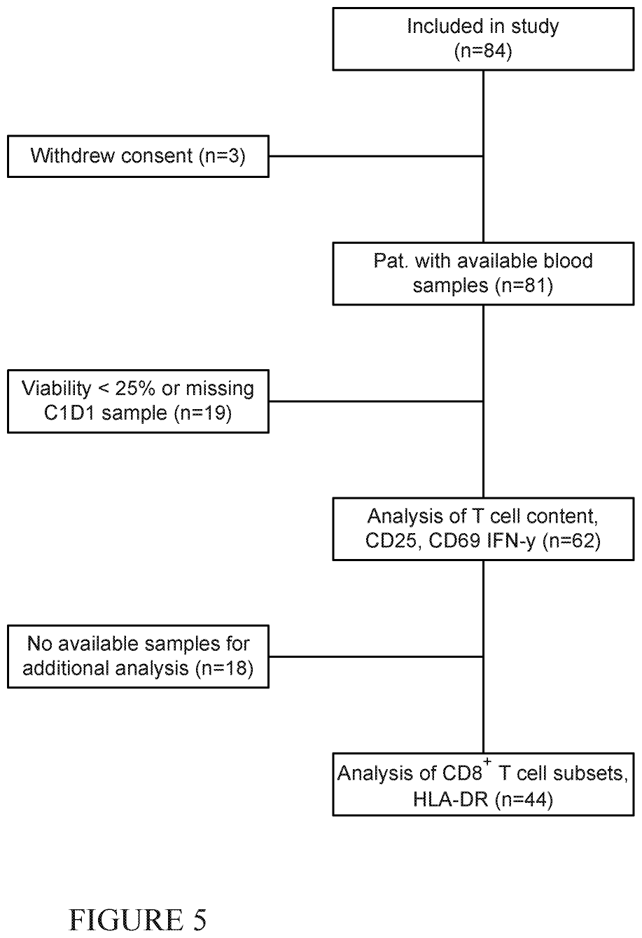

Blood samples were available from 81 out of 84 patients. Differential counts of whole blood were performed at local sites and were utilized to calculate absolute counts of blood CD8+ T cells. All available samples were analyzed for T cell content and expression activation markers (including CD25 and CD69 and IFN-γ production in response to PMA). If an analysis failed according to pre-defined criteria (experimental failure, few cells, poor cellular viability), a second sample was thawed for re-analysis. If also the second attempt failed to generate data, these samples were excluded from analysis. In a second set of experiments, available samples were analyzed for distribution of T cell subsets and HLA-DR expression. All successfully analyzed samples, according to the pre-defined criteria stated above, were included in this report. A flow chart of patients that were included or excluded from the analyses is shown in FIG. 5.

IFN-γ-Production after PMA/Ionomycin or Peptide Stimulation

Thawed samples collected before and after the first and third treatment cycles of HDC/IL-2 immunotherapy were seeded in Iscoves' medium supplemented with 10% FCS in 96-well plates, 1×10⁶ cells per well. Cells were left to rest over night at 37° C. The next day, cells were washed with warm medium before stimulation consisting either of a 5 hours incubation with 0.2 μg/ml PMA (Sigma-Aldrich Munich, Germany) together with 2 μg/ml ionomycin (Sigma-Aldrich) or a 6 hours incubation with a pool of AML-peptides (overlapping peptides covering the leukemia-associated proteins WT1, PRAME, survivin and hTERT; Miltenyi Biotec) or, as a control, CEF-peptides (32 peptides specific for MHC class I with sequences derived from human cytomegalovirus (HCMV), Epstein-Barr virus (EBV) and influenza viruses; Miltenyi Biotec).

The final concentration of each peptide was 0.6 nmol. The co-stimulatory molecules anti-CD28 (CD28.2; BD Biosciences) and anti-CD49d (9F10; BD Biosciences), 2 μg/ml of each, were added to wells with peptide stimulation, as well as to the negative control samples, which served to determine the background signal. For all stimuli, Golgiplug (BD Biosciences) was added during the last 4 hours of stimulation according to manufacturer's protocol. Samples were stained with LIVE/DEAD fixable yellow stain and surface markers before being fixed, permeabilized and intracellularly stained with IFN-γ as described above. Patients were considered to harbor AML- or CEF-specific CD8+ T cells if the frequency of IFN-γ-producing CD8+ T cells exceeded 0.05% (after subtraction of background as determined by the negative control sample) at any of the time points (C1D1, C1D21, C3D1, C3D21). To avoid selection bias, only patients with no events (relapses) occurring before the last time point (C3D21; 105 days) were considered in these analyses.

### Statistics

In accordance with the statistical analysis plan, paired t-test was used for single comparisons of CD8+ T cell phenotypes. To determine the impact of the dynamics of CD8+ subsets or markers on outcome, samples were dichotomized by the median for single time points or by induction/reduction during the first treatment cycle if not otherwise stated. The analyses of T cell function vs. outcome are based on data for LFS, defined as the time in days from start of immunotherapy with HDC/IL-2 to relapse or death from any cause, and OS, defined as the corresponding time to death, available at the trial closing date (Oct. 13, 2014), i.e. when all patients had been followed for at least 24 months (18 months of treatment and 6 months of additional follow-up). Relapse was defined as at least 5% blast cells in the bone marrow or presence of extramedullary leukemia. The impact of T cell phenotype on LFS and OS was analyzed using the log-rank test.

The impact of age, risk group classification according to recommendations by the European LeukemiaNet [Blood. 2010; 115(3):453-474], number of induction courses required to achieve CR (1 or >1), and number of consolidation courses (0-2 or >2) on LFS and OS was assessed using the Cox univariate regression model. The prognostic factors with a p value below 0.1 (age, and number of induction cycles) were included as potential confounders in a Cox multivariate regression analysis (Table 2).

TABLE 2
Univariate and multivariate Cox regression analyses of the impact of
aspects of CD8⁺ T cell phenotype on LFS and OS.

| | Univariate | | | Multivariate* | | |
|---|---|---|---|---|---|---|
| | | Conf. | | | Conf. | |
| Cox regression analysis | HR | Interval | p-value | HR | Interval | p-value |
| All patients (n = 44) | | | | | | |
| Induction T_{N} VS LFS | 0.45 | 0.20-0.98 | 0.04 | 0.47 | 0.21-1.06 | 0.07 |
| Induction T_{N} VS OS | 0.29 | 0.10-0.82 | 0.02 | 0.29 | 0.10-0.85 | 0.02 |
| Induction T_{CM} Vs LFS | 1.08 | 0.50-2.36 | 0.84 | 1.46 | 0.64-3.32 | 0.37 |
| Induction T_{CM} VS OS | 1.43 | 0.52-3.93 | 0.49 | 2.65 | 0.91-7.76 | 0.08 |
| Reduction T_{EM} VS LFS | 0.26 | 0.12-0.60 | 0.001 | 0.26 | 0.11-0.60 | 0.002 |
| Reduction T_{EM} VS OS | 0.24 | 0.08-0.69 | 0.009 | 0.23 | 0.08-0.70 | 0.01 |
| Induction T_{EFF} VS LFS | 0.50 | 0.23-1.09 | 0.08 | 0.46 | 0.20-1.03 | 0.06 |
| Induction T_{EFF} VS OS | 0.35 | 0.12-0.99 | 0.048 | 0.27 | 0.09-0.80 | 0.02 |
| Transition vs LFS ** | 0.19 | 0.07-0.50 | 0.001 | 0.17 | 0.06-0.47 | 0.001 |
| Transition vs OS ** | 0.13 | 0.03-0.59 | 0.008 | 0.12 | 0.02-0.54 | 0.006 |
| Patients >60 yo (n = 27) | | | | | | |
| Induction T_{N} VS LFS | 0.25 | 0.88-0.68 | 0.007 | 0.20 | 0.07-0.61 | 0.005 |
| Induction T_{N} VS OS | 0.17 | 0.04-0.67 | 0.01 | 0.13 | 0.03-0.59 | 0.008 |
| Induction T_{CM} VS LFS | 1.39 | 0.50-3.85 | 0.53 | 1.41 | 0.50-3.97 | 0.52 |
| Induction T_{CM} Vs OS | 3.37 | 1.01-11.2 | 0.048 | 3.77 | 1.08-13.2 | 0.04 |
| Reduction T_{EM} VS LFS | 0.10 | 0.03-0.38 | 0.001 | 0.10 | 0.03-0.39 | 0.001 |
| Reduction T_{EM} VS OS | 0.14 | 0.03-0.57 | 0.006 | 0.13 | 0.03-0.57 | 0.006 |
| Induction T_{EFF} Vs LFS | 0.22 | 0.07-0.68 | 0.009 | 0.26 | 0.08-0.87 | 0.03 |
| Induction T_{EFF} VS OS | 0.10 | 0.03-0.40 | 0.001 | 0.10 | 0.02-0.51 | 0.005 |
| Transition vs LFS ** | <0.001 | *** | | <0.001 | *** | |
| Transition vs OS ** | <0.001 | *** | | <0.001 | *** | |

Abbreviations: LFS, leukemia-free survival, OS, overall survival
*Adjusted for age and number of induction cycles
** Transition: reduction of TEM with simultaneous induction of TEFF.
*** To few events for Cox regression analysis

All indicated P-values are 2-sided. This study was conducted according to Declaration of Helsinki principles. The trial was approved by the Ethics Committees of each participating institution, and all patients gave written informed consent before enrollment.

### Example 4

### Dynamics of Cytotoxic T Cell Subsets During Immunotherapy Predicts Outcome in Acute Myeloid Leukemia—Results

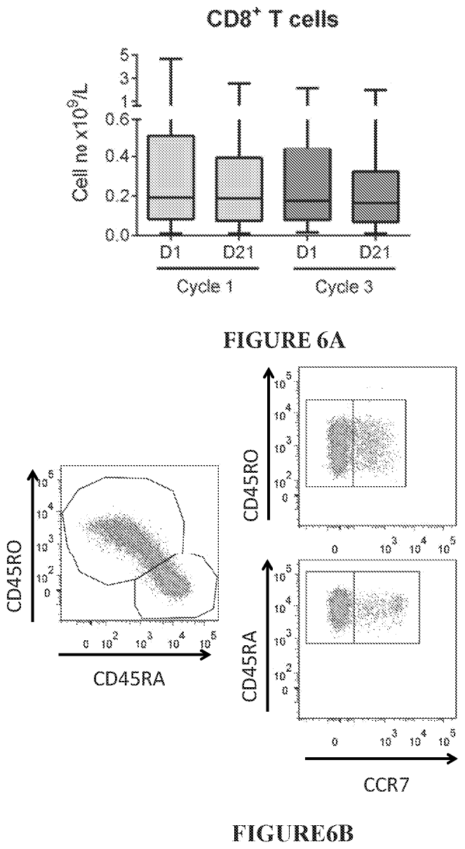

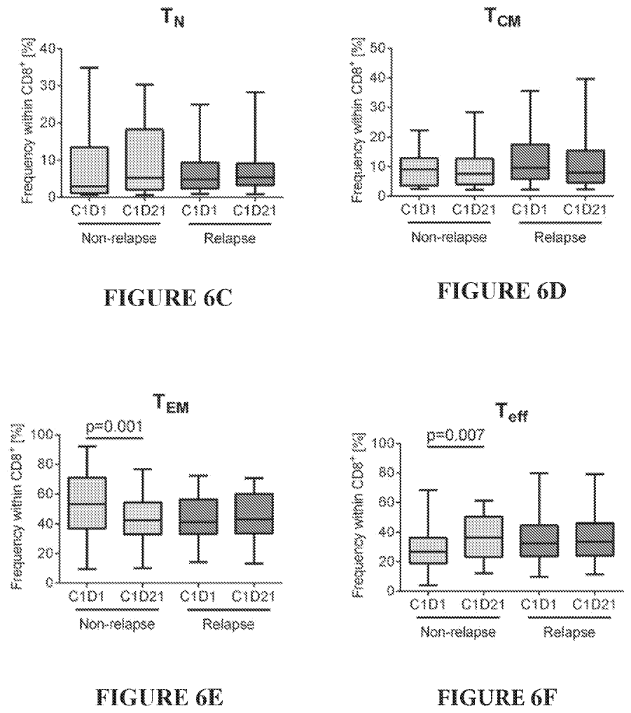

AML patients in first CR received 10 consecutive 3-week cycles of HDC/IL-2 in the post-consolidation phase, as outlined in FIG. 4. Peripheral blood collected before and after the first and the third cycles of immunotherapy was analyzed for CD8+ T cell content and phenotype. During the treatment cycles, HDC/IL-2 did not alter the absolute counts of CD8+ T cells in blood (FIG. 6A). Also, the CD8+ T cell counts (above or below the median) before or after therapy did not impact on relapse risk (data not shown).

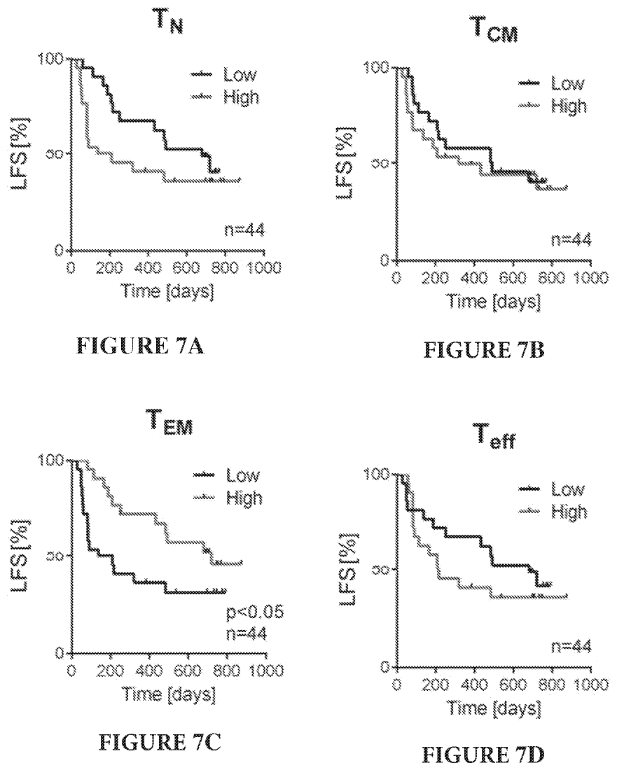

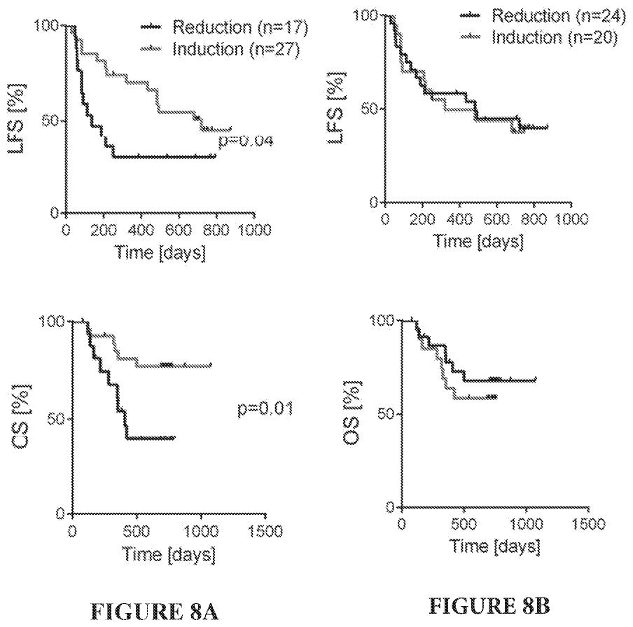

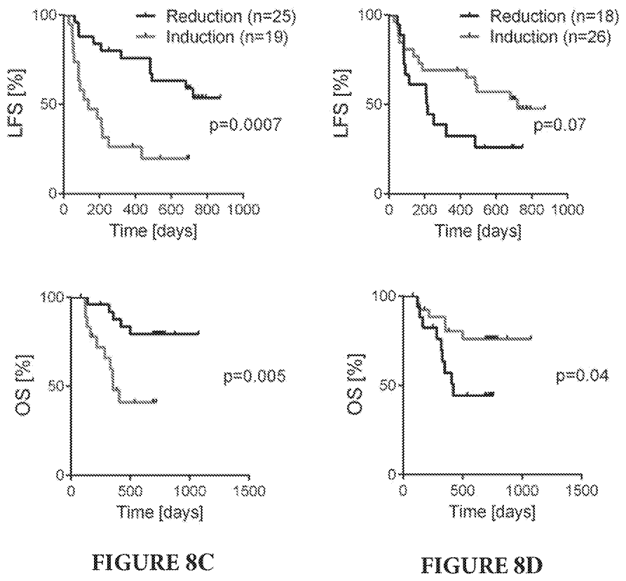

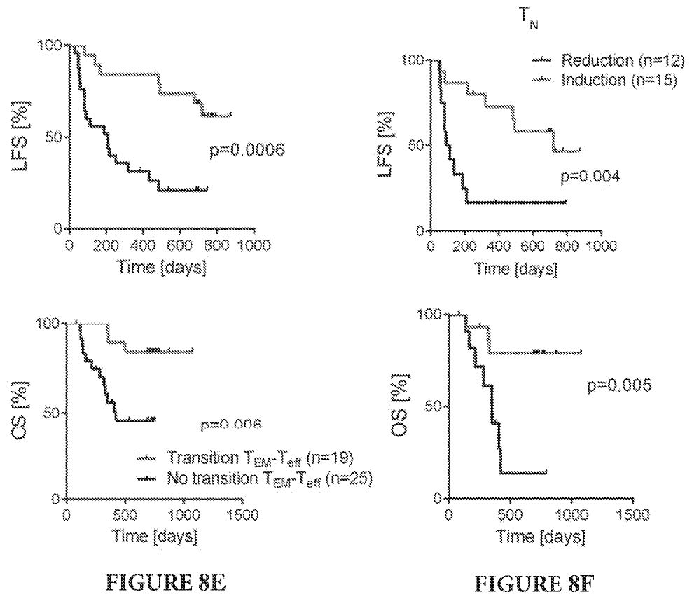

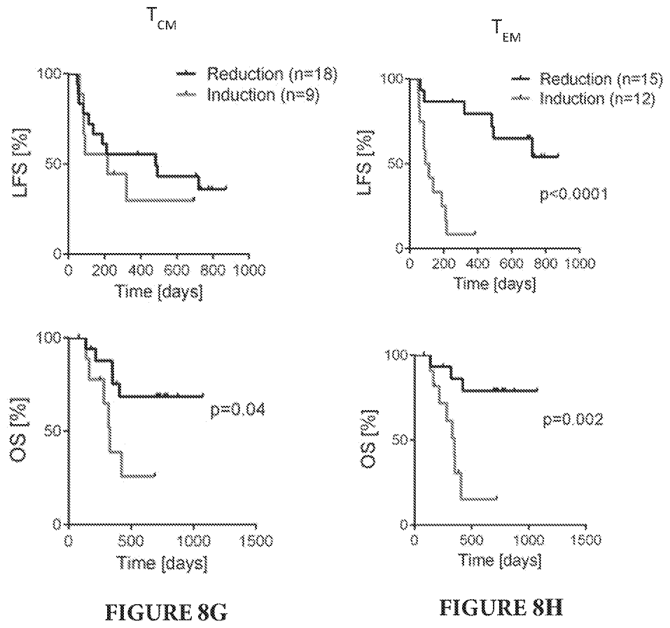

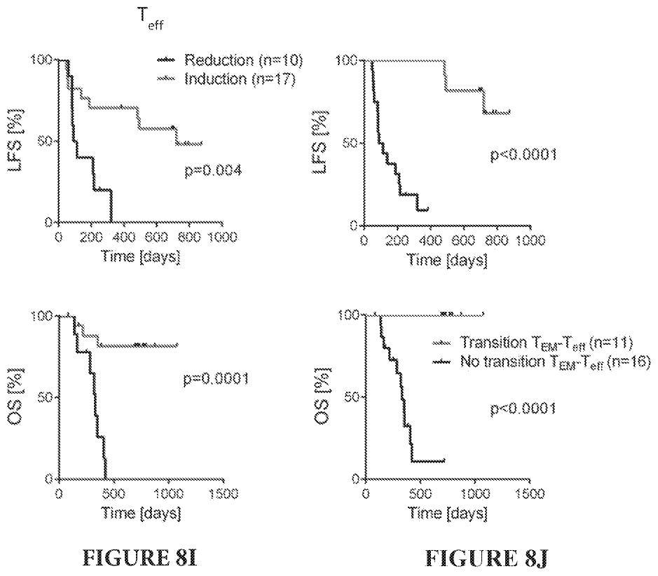

When comparing the distribution of CD8+T_{N}, T_{CM}, T_{EM} and T_{eff} before and after the first treatment cycle, non-relapsing patients showed a distinct reduction of the fraction of T_{EM} cells along with an induction of T_{eff} cells (FIG. 6). At onset of therapy, patients with a high percentage (above the median) of T_{EM} cells showed a slightly higher likelihood of LFS, while no significant differences in relapse risk were found for patients with a high or low percentage (by the median) of T_{N}, T_{CM}, or T_{eff} cells (FIG. 7). Patients experiencing a reduction of the frequency of T_{EM} cells or an induction of T_{eff} cells during cycle 1 showed significantly improved LFS and/or OS (FIG. 8C-D with results of multivariate analyses shown in Table 2). Also, induction of the frequency of naïve T cells during the first treatment cycle significantly predicted LFS and OS (FIG. 8A, Table 2). The distribution of T_{CM} cells was seemingly unaltered during immunotherapy (FIG. 8D) and did not influence relapse risk (FIG. 8B). The distribution of T cell subsets was not significantly altered during treatment cycle 3 (data not shown).

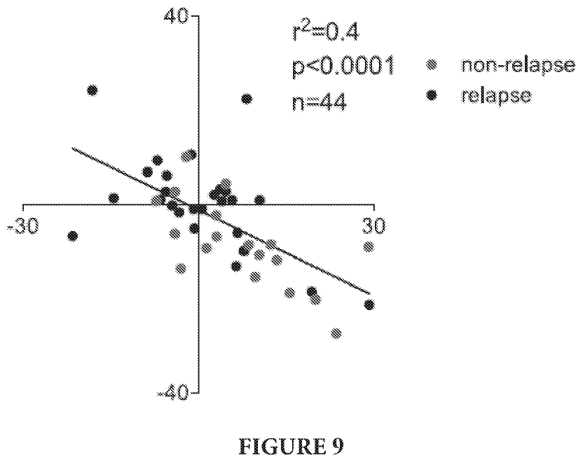

Stimulation of CD8+ T cells with antigen has been proposed to drive differentiation from T_{N}→T_{CM}→T_{EM}→T_{eff} cells [Current opinion in immunology. 2013; 25(5):556-563; Vaccine. 2015; 33(7):914-923]. We thus speculated that T_{eff} cells were induced from T_{EM} cells during the first treatment cycle of HDC/IL-2. Indeed, a correlation was observed between an induction of T_{eff} and a reduction of T_{EM} during cycle 1 (FIG. 9). Eighteen of out 44 analyzable patients (41%) showed a memory to effector T cell transition, defined as a reduction of CD8+T_{EM} cells with a concomitant increase in CD8+T_{eff} cells. These patients showed improved LFS (HR 0.19, P<0.001) and OS (HR 0.13, P=0.002; FIG. 8E, Table 2).

### T Cell Phenotypes in Older Patients

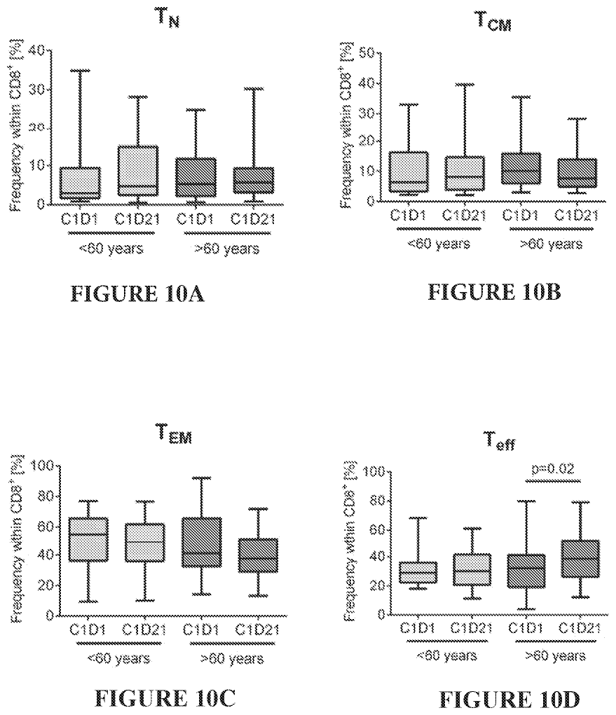

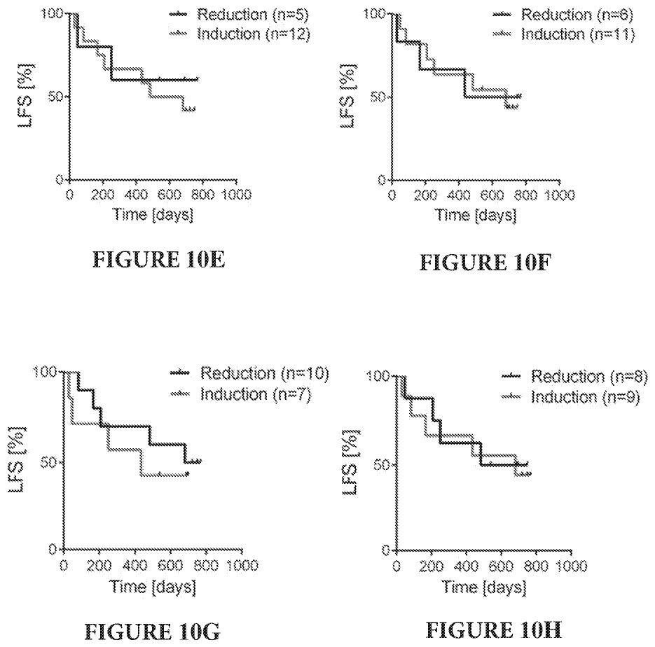

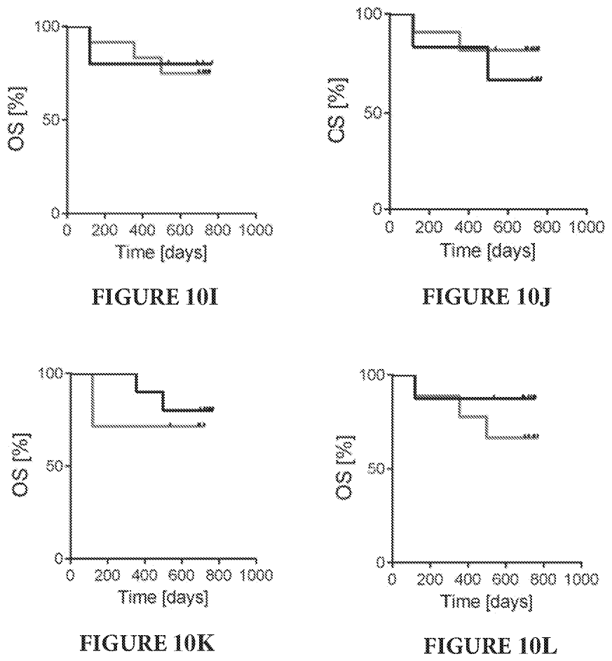

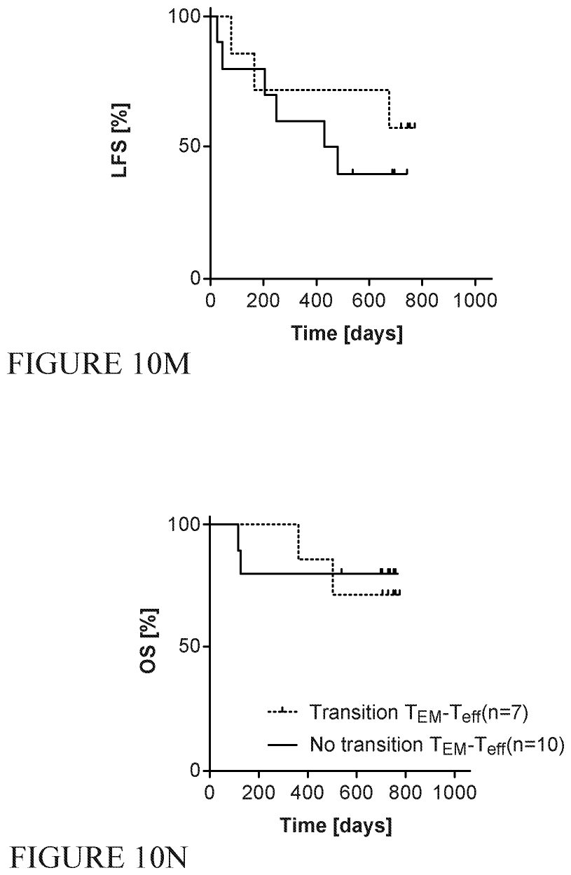

The trial protocol specified analyses of outcome by subgroups according to patient age at enrollment (<60 and >60 years). The CD8+ T cell count or the distribution of CD8+T_{N}/T_{CM}/T_{EM}/T_{eff} cells at onset of immunotherapy did not differ significantly between age groups (FIG. 10A-D). During the first cycle, treatment with HDC/IL-2 induced a significant increase of the frequency of T_{eff} cells only in older patients (FIG. 10D). The impact of the altered distribution of T_{N} and T_{eff} cells, the reduction of the frequency of T_{EM} cells and the apparent transition of T_{EM} cells into T_{eff} cells on outcome was pronounced in older patients. All of these aspects of immunotherapy-induced CD8+ T cell differentiation thus heralded LFS and/or OS in this age group (FIGS. 8F-J) and remained significantly predictive after correction for prognostic factors (Table 2). The dynamics of CD8+ T cell subsets during cycle 1 did not significantly prognosticate LFS or OS in younger patients (FIGS. 10E-N).

### Additional Markers of T Cell Activation

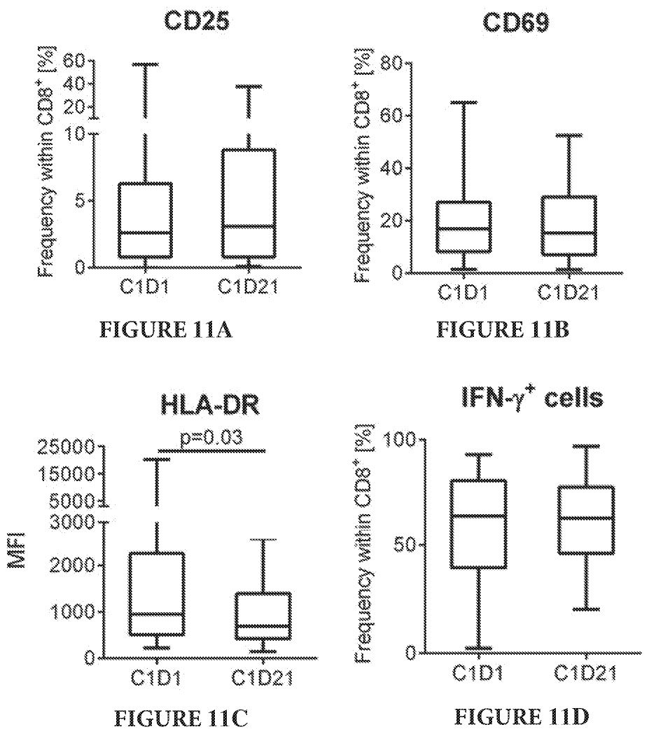

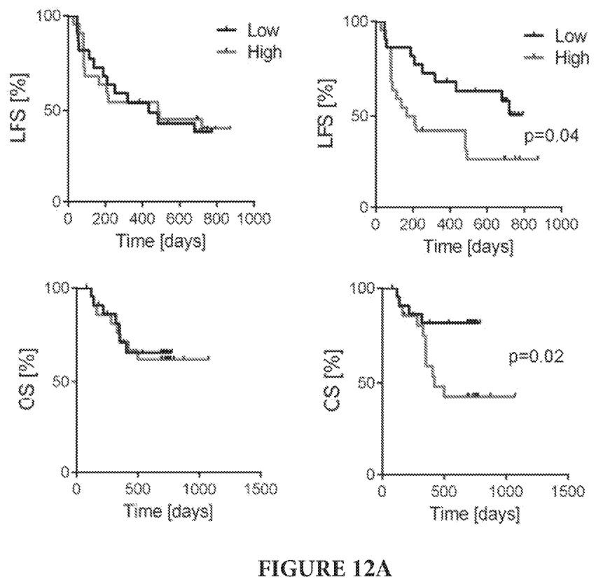

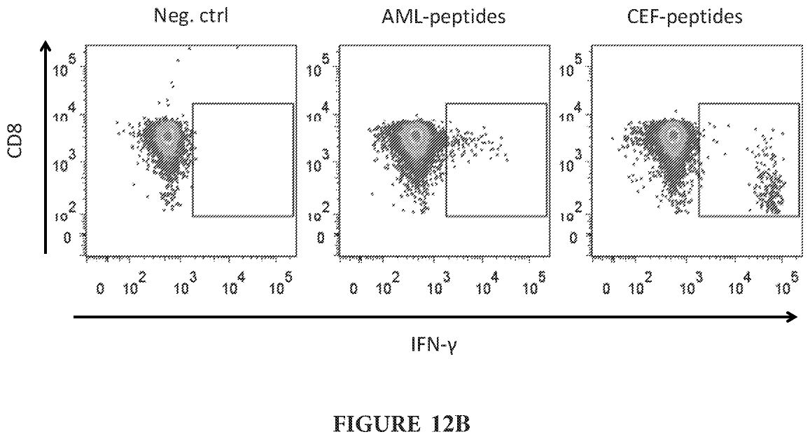

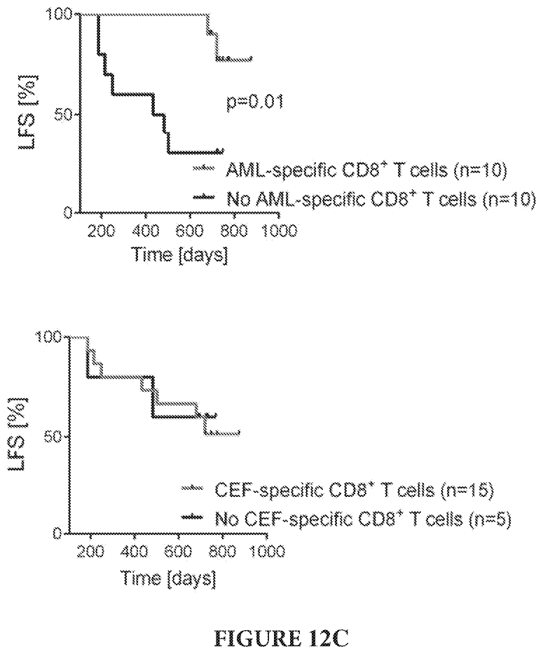

In addition to the distribution of T cell phenotypes we analyzed the impact of the immunotherapy on cytotoxic T cell activation markers. The expression levels of CD69 and CD25, which are frequently employed markers of activated T cells, were unaffected by HDC/IL-2 treatment (FIGS. 11A-B) and the expression levels of these markers did not influence clinical outcome (data not shown). In contrast, the expression of HLA-DR was reduced during the first cycle of therapy (FIG. 11C). HLA-DR was mainly expressed by the memory populations, and the reduction was significant for T_{CM} and T_{EM} cells but not T_{N} or T_{eff} cells (data not shown). A low expression of HLA-DR on CD8+ T cells at the end of the first HDC/IL-2 treatment cycle weakly predicted a favorable clinical outcome (univariate Cox regression analysis for OS was P=0.04, while multivariate Cox regression analysis for OS was P=0.12; FIG. 12A).

### Presence of Leukemia-Specific T Cells Heralds Maintained CR

We next determined the ability of CD8+ T cells to produce IFN-γ ex vivo before and after immunotherapy. The capacity of patients' CD8+ T cells to produce IFN-γ after stimulation with PMA/ionomycin was similar before and after the first treatment cycle (FIG. 11D) and did not impact on the clinical outcome (not shown). To determine whether patients harbored CD8+ T cells that were specifically reactive with leukemic antigens, PBMCs were stimulated by peptide pools representing known leukemia-associated antigens (WT1, survivin, PRAME and hTERT) followed by quantification of IFN-γ-producing CD8+ T cells. Healthy donor CD8+ T cells from PBMCs did not produce above background levels of IFN-γ in response to the leukemia-derived peptides (data not shown). Three out of 20 analyzed patients displayed antigen-specific CD8+ T cells against any of these antigens at onset of immunotherapy (C1D1). Two of these patients experienced late relapses (at >600 days). Seven patients acquired leukemia-reactive T cells during immunotherapy (at C1D21, n=2, C3D1, n=4 or C3D21, n=1), all of whom remained in uninterrupted CR. By Kaplan-Meier analysis, presence of leukemia-specific CD8+ T cells predicted LFS (P=0.01) whereas presence of antigen-specific CD8+ T cells responding to viral control peptides (CMV, EBV and influenza; CEF) did not (P=0.5; FIG. 12B-C).

The results of this study indicate, for the first time, that an altered distribution of cytotoxic T cell phenotypes in blood during immunotherapy is relevant to the prognosis of non-transplanted AML patients in CR. A major finding was that these aspects of T cell immunity determined the relapse risk and survival of older patients, who are at high risk of relapse and death. Evaluating memory to effector T cell transition may be broadly useful in T cell-based cancer immunotherapy. The reason for the lack of significant correlation between the dynamics of CD8⁺ T cell subsets and outcome in younger patients is not known, but might be related to a lower incidence of relapse in this age group along with fewer samples available for analysis.

The precise mechanism explaining our finding of a shift from T_{EM} cells to T_{eff} cells in blood of AML patients during the first cycle of HDC/IL-2 immunotherapy remains to be determined. However, IL-2 has been reported to promote the development of CD8⁺ T cells into memory and effector cell populations, and it is thus conceivable that the IL-2 component of the HDC/IL-2 regimen was crucial for the observed memory to effector T cell transition. Also, the memory to effector cell transition is compatible with the view that T_{EM} cells differentiate into T_{eff} cells after antigen exposure. While alternative explanations are possible, including extravasation of T cell subsets during immunotherapy, we hypothesize that immunotherapy with HDC/IL-2 facilitates the development of effector T cells, which may explain the strong prediction of clinical outcome in patients experiencing T_{EM} to T_{eff} transition. Of note, others have shown that AML-specific T cells carry a T_{eff} cell phenotype. In further support for the development of functional T cell immunity during immunotherapy, detectable levels of CD8⁺ T cells that reacted with leukemic peptides were noted in 7/20 patients during the course of therapy. Presence of leukemia-specific CD8⁺ T cells, but not CD8⁺ T cells reactive with common viral antigens, significantly predicted LFS. These results concur with previous observations on a role for immunoreactive leukemia-associated antigens in AML and lend support to the role of cytotoxic T cells for surveillance of the leukemic clone.

HLA-DR is considered a T cell activation marker, but increased expression of HLA-DR on CD8⁺ T cells has also been linked to T cell suppression and exhaustion in cancer, chronic virus infections and aging. The reduction of HLA-DR expression in T_{CM} and T_{EM} cells during immunotherapy and the trend towards favorable clinical outcome among patients with reduced expression suggest that the memory population of CD8⁺ T cells may be shifted towards improved effector function during immunotherapy.

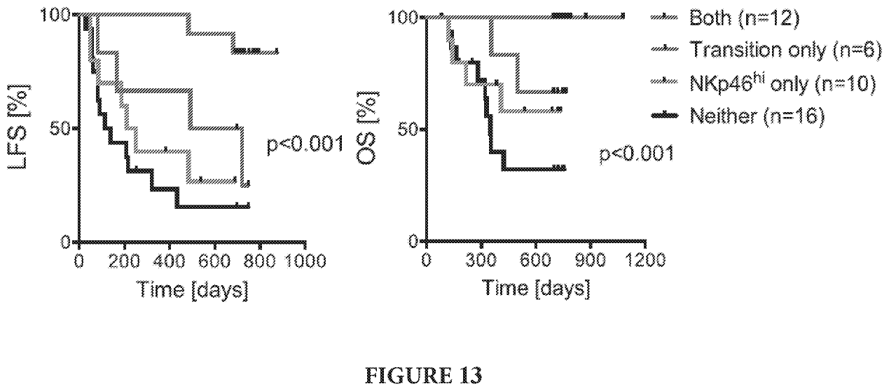

HDC/IL-2 has been developed for AML immunotherapy to expand and activate populations of T cells and natural killer (NK) cells (IL-2 component) and concurrently protect these anti-leukemic effector cells against inactivation by myeloid cell-derived reactive oxygen species (HDC component). Earlier results from the Re:Mission trial show that treatment with HDC/IL-2 triggered expression of natural cytotoxicity receptors (NCR), which are activating receptors of importance for NK cell recognition of aberrant cells, along with increasing NK cell counts in blood. The Re:Mission trial results suggested that in-cycle increments of NCR expression and NK cell counts only weakly predicted LFS and OS, whereas the results presented herein imply that altered distribution of CD8⁺ T cell subsets during the first treatment cycle was critical to prognosis. We observed that patients with a T_{EM} to T_{eff} cell transition during the first treatment cycle along with high NKp46 expression on cytotoxic CD16⁺ NK cells after immunotherapy were strikingly protected from relapse, while patients with T_{EM} to T_{eff} cell transition or high NKp46 expression alone were only partly protected (FIG. 13). These data support the hypothesis that T and NK cell effector functions evolve simultaneously during immunotherapy and that both cell subsets may contribute in relapse protection.

### Example 5

### NK Cell Expression of Natural Cytotoxicity Receptors May Determine Relapse Risk in Older AML Patients Undergoing Immunotherapy for Remission Maintenance

At diagnosis, approximately 70% of patients with acute myeloid leukemia (AML) are >60 years old. While a high proportion of older patients achieve complete remission (CR) after chemotherapy, leukemia relapse is common in the post-chemotherapy phase and significantly explains why the rates of 5-year survival of older patients are in the range of 10-15%. Immunotherapy with histamine dihydrochloride and low-dose interleukin-2 (HDC/IL-2) aims at boosting anti-leukemic functions of natural killer (NK) cells to reduce or eradicate residual leukemia. In a phase III trial, treatment with HDC/IL-2 was shown to prevent relapse in AML patients in CR.

Several aspects of NK cell function are reportedly relevant to AML prognosis, but a systematic analysis of aspects of NK cell biology in older patients has not been carried out. Human NK cells comprise two main phenotypes: the cytotoxic CD16⁺/56+NK cells (here referred to as CD16⁺ NK cells) constitute 90-95% of blood NK cells in healthy subjects, whereas the weakly cytotoxic CD16⁻/56^{bright} cells (CD56^{bright} NK cells) are regarded as precursors of CD16⁺ NK cells. NK cell cytotoxicity is regulated by activating and inhibitory NK cell receptors and their cognate ligands on malignant target cells. The main activating receptors comprise the natural cytotoxicity receptors (NCRs; NKp46, NKp30 and NKp44) and NKG2D. We have previously published an interim report of aspects of NK cell biology in AML patients who participated in a phase IV trial using HDC/IL-2 for remission maintenance. Here, we report final results from this trial, with a focus on the subgroup of older AML patients (>60 years).

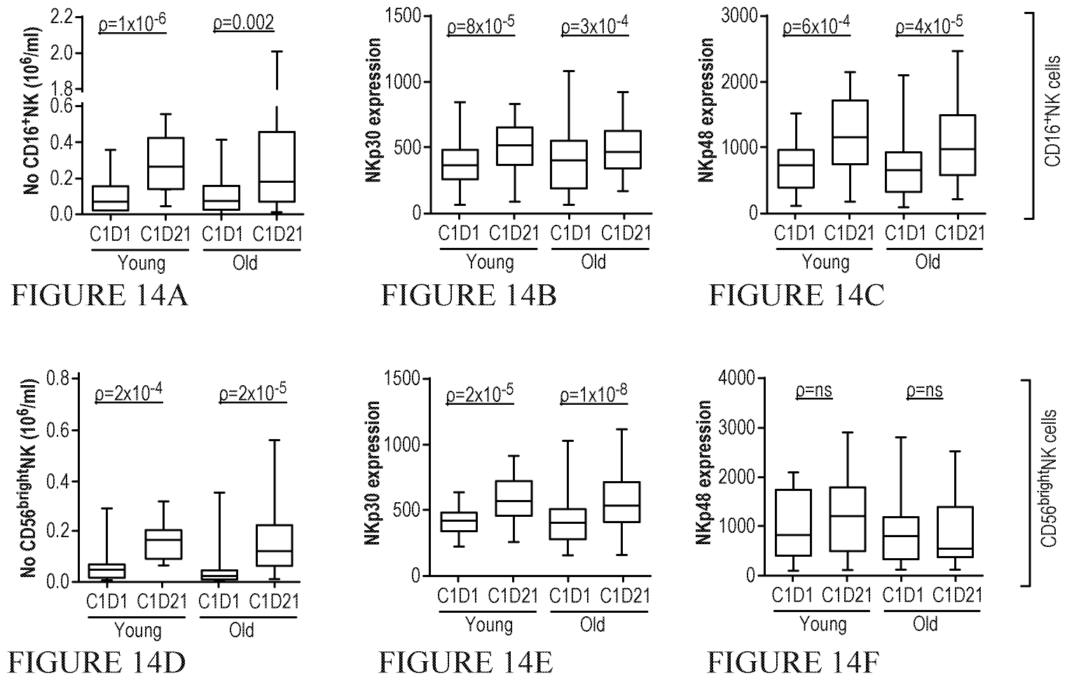

AML patients in first CR received ten three-week cycles of HDC/IL-2 over 18 months as described elsewhere. Blood samples were drawn before and after the first cycle for analysis of absolute NK cell counts and NK cell expression of NCRs. A previous interim report showed that treatment with HDC/IL-2 augmented NK cell counts in the blood along with an increased NK cell expression of NKp30 and NKp46 during treatment cycles. We first compared the degree of NK cell and NCR induction in younger (<60 years) and older (>60 years) patients, respectively. The NK cell counts and NCR expression at onset of treatment did not differ between these age groups with the exception that the ratio of CD56^{bright} to CD16⁺ cells was lower in older patients (P=0.045, Mann-Whitney Test), which is in agreement with previous findings in healthy subjects. Younger and older patients did not differ regarding the accumulation of CD16⁺ and CD56^{bright} NK cells in the blood during treatment cycles (FIG. 14A, 14D) or induction of NK cell expression of NKp30 and NKp46 receptors (FIG. 14B-14C, 14E-14F).

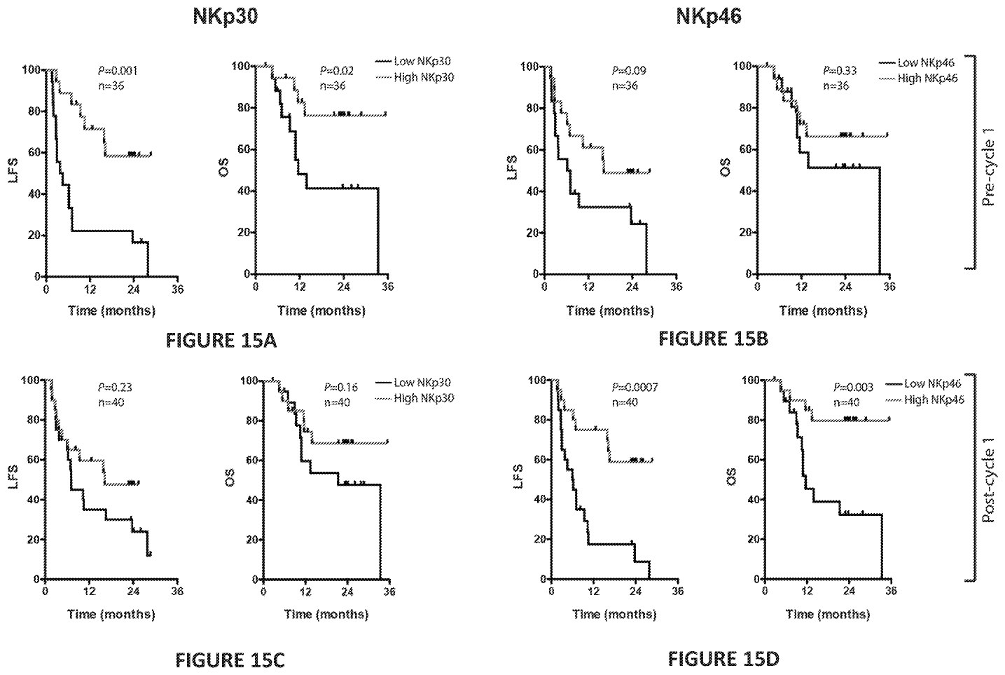

We aimed at determining the impact of NK cell-related markers on outcome by dichotomizing younger and older patients by high or low (by the median) NK cell counts or NK cell NCR expression intensity followed by analysis of leukemia-free survival (LFS) and overall survival (OS). NK cell counts did not significantly predict outcome in younger or older patients, and NK cell NCR expression did not predict outcome in younger patients (P>0.5 for LFS, not shown). Older patients with an above-median expression of NKp30 on CD16⁺ NK cells at the onset of therapy (cycle 1 day 1; C1D1) showed improved LFS and OS, with a similar trend for NKp46 (FIGS. 15A and 15C). A high expression of NKp46 on CD16⁺ NK cells after the first treatment cycle (cycle 1 day 21; C1D21) was positively associated with LFS and OS with a similar trend for NKp30 (FIGS. 15B and 15D). No significant associations were observed between outcome and the level of induction of NK cell counts or the induction of NCRs during the first treatment cycle (ie. C1D21 minus C1D1 levels, not shown). In multivariate analyses corrected for age, risk group classification, number of induction courses required to achieve CR and number of consolidation courses, NKp30 expression on C1D1 and NKp46 expression on C1D21 independently predicted LFS and/or OS in older patients (Table 3). One patient in CR died of sepsis whereas all other deaths were preceded by a relapse, which illustrates the impact of relapse for survival in the post-consolidation phase also in the group of elderly AML patients.

The reason for the lack of correlation between NCR expression and outcome in younger patients is not known, but may relate to the lower number of younger patients in the study, along with a lower incidence of relapse and death in this age group (data not shown). NK cell function relies on interactions between NCR and their ligands on target cells with ensuing activation of NK cell cytotoxicity. While NCR ligands are frequently expressed by malignant AML cells, NK cells of newly diagnosed AML patients may express lower densities of NCR, which impacts on the anti-leukemic efficiency of NK cells as well as on survival and the likelihood of achieving CR after chemotherapy. Our results extend these previous findings by pointing to a role for NCR in preventing relapse in the post-chemotherapy phase of AML. Further studies are required to confirm these results and to establish whether NK cell NCR expression heralds relapse also in untreated patients or in patients undergoing other immunotherapies.

TABLE 3
Univariate and multivariate analyses of the impact of NKp30 or
NKp46 expression on LFS and OS.

| | Univariate analysis | | | Multivariate analysis | | |
|---|---|---|---|---|---|---|
| | Hazard | Confidence | | Hazard | Confidence | |
| Variable | ratio | interval | p-value | ratio | interval | p-value |
| NKp30, C1D1, LFS | 0.24 | 0.099-0.61 | 0.002 | 0.14 | 0.043-0.43 | 0.001 |
| NKp30, C1D1, OS | 0.28 | 0.088-0.91 | 0.034 | 0.17 | 0.037-0.78 | 0.022 |
| NKp46, C1D21, LFS | 0.25 | 0.10-0.59 | 0.001 | 0.28 | 0.093-0.82 | 0.020 |
| NKp46, C1D21, OS | 0.21 | 0.067-0.66 | 0.007 | 0.22 | 0.047-1.05 | 0.058 |

Univariate and multivariate Cox regression analyses were utilized to determine the impact of above or below median expression levels of NKp30 cycle 1 day 1 (C1D1) or NKp46 cycle 1 day 21 (C1D21) on LFS and OS in patients >60 years old. In the multivariate analyses, hazard ratios were corrected for age, risk group classification, number of induction courses needed to achieve CR, and number consolidation courses

### Materials and Methods

### Patients

This single-armed multicenter phase IV study (Re:Mission, NCT01347996, registered at www clinicaltrials.gov) enrolled 84 patients (age 18-79) with AML in first CR who received ten 21-day cycles of HDC/IL-2 for 18 months or until relapse or death. Primary endpoints included assessment of the quantitative and qualitative pharmacodynamic effects of HDC/IL-2 by monitoring NK cell phenotypes and their functionality before and after the first treatment cycle. The protocol stated that data collected in support of these objectives were to be analyzed for the defined populations as a whole and by subgroups according to patient age at enrollment (<60 and >60 years). The Analyses of NK cell counts and NCR expression vs. outcome were performed post-hoc. The characteristics at enrollment of older patients (>60 years, median 67.2, range 60-79) are accounted for in Table 4. Details of patient characteristics, induction and consolidation therapy, exclusion criteria, treatment and dosing are found in a previous interim report (Example 4).

### Sampling of Peripheral Blood and Flow Cytometry

Peripheral blood was collected before and after treatment cycle 1, ie. on day 1 and day 21 of cycle 1 (C1D1 and C1D21), and PBMC were isolated and cryopreserved at local sites and shipped on dry ice to the central laboratory (at the Sahlgrenska Cancer Center, University of Gothenburg, Sweden) for flow cytometry analysis. PBMC samples were stained with fluorochrome-conjugated antibodies and a viability marker and analyzed using a 4-laser BD LSRFortessa SORP (BD Biosciences, San Diego, Calif.), as accounted for in detail elsewhere (Example 4).

Samples were available from 32 out of 37 younger patients and from 45 out of 47 older patients. All available samples were analyzed. If an analysis failed according to pre-defined criteria (experimental failure, few cells, poor cellular viability), a second sample was thawed for re-analysis. In 18 cases for C1D1 samples and in 12 cases for C1D21 samples, also the second attempt failed to generate data, and these samples were excluded from analysis. Differential counts of whole blood were performed at local sites and were utilized to calculate absolute counts of blood NK cells. Differential counts were lacking from four younger and five older patients.

TABLE 4
Patient characteristics (age >60)

| | n (%) | LFS (%) |
|---|---|---|
| Sex | | |
| Female | 23 (49) | 6/23 (26) |
| Male | 24 (51) | 10/24 (42) |
| Risk group | | |
| Favorable risk | 17 (36) | 8/17 (47) |
| Intermediate I | 10 (21) | 2/10 (20) |
| Intermediate II | 9 (19) | 4/9 (44) |
| High risk | 6 (13) | 1/6 (17) |
| ND | 5 (11) | 1/5 (20) |
| Karyotype | | |
| Normal | 23 (49) | 9/23 (39) |
| Favorable | 5 (11) | 2/5 (40) |
| Unfavorable | 6 (13) | 2/6 (33) |
| Other | 10 (21) | 3/10 (30) |
| ND | 3 (6) | 0/3 (0) |
| Mutation status | | |
| NPM1 (n = 39) | 14 (36) | 6/14 (43) |
| FLT3 (n = 37) | 3 (8) | 0/3 (0) |
| CEBPA (n = 23) | 1 (4) | 0/1 (0) |
| Induction courses (n = 46) | | |
| 1 | 32 (70) | 14/32 (44) |
| >1 | 14 (30) | 2/14 (14) |
| Consolidation courses (n = 46) | | |
| 0-2 | 26 (57) | 6/26 (23) |
| >2 | 20 (43) | 10/20 (50) |

### Statistics

In accordance with the statistical plan, paired/−test was used for single comparisons of NK cell phenotypes. The analyses of NK cell markers vs. outcome are based on data for LFS, defined as the time in days from start of immunotherapy with HDC/IL-2 to relapse or death from any cause) and OS available at the trial closing date (Oct. 13, 2014), i.e. when all patients had been followed for at least 24 months (18 months of treatment and 6 months of additional follow-up). Relapse was defined as at least 5% blast cells in the bone marrow or by the occurrence of extramedullary leukemia. LFS was defined as the time from the first day of treatment with HDC/IL-2 to relapse or death from any cause. OS was defined as the corresponding time to death regardless of cause. LFS and OS were analyzed using the log-rank test. Parameters that significantly predicted LFS and/or OS were further analyzed by univariate and multivariate Cox regression analysis. In the multivariate analyses, hazard ratios were corrected for age, risk group classification, number of induction courses required to achieve CR (1 or >1) and number of consolidation courses (0-2 or >2; Table 1). All indicated P-values are 2-sided. Patients were risk-classified according to recommendations by the European LeukemiaNet. The trial was approved by the Ethics Committees of each participating institution, and all patients gave written informed consent before enrollment.

### Example 6

### Dynamics of Myeloid Cell Populations During Relapse-Preventive Immunotherapy in Acute Myeloid Leukemia

In patients with acute myeloid leukemia (AML) immature myeloid cells rapidly accumulate in blood and bone marrow. Despite that the majority of AML patients achieve disappearance of leukemic cells (complete remission, CR) after chemotherapy, most adult patients experience relapse of leukemia with poor prospects for long-term survival Immunotherapy with histamine dihydrochloride (HDC) in conjunction with the T and NK cell-activating cytokine IL-2 (HDC/IL-2) is currently used within the EU and in Israel for relapse prevention in the post-consolidation phase. The approval of this regimen was based on phase III trial results showing a significantly reduced relapse risk in patients receiving HDC/IL-2, and meta-analyses comparing relapse prevention achieved by IL-2 alone or HDC/IL-2 support the clinical effectiveness of the HDC component.

NOX2 is the reactive oxygen species (ROS)-generating NADPH oxidase of myeloid cells. HDC targets histamine H₂-receptors (H₂Rs) on mature myeloid cells to suppress NOX2-dependent ROS formation. Inhibition of NOX2-derived ROS has been found to protect adjacent anti-leukemic effector cells from ROS-induced inactivation, which has been proposed as a mechanism of relevance for the relapse-preventive properties of HDC in AML. In addition, previous studies implicate HDC as a pro-differentiating agent for myeloid cells. Yang et al. thus reported that mice with histamine deficiency due to genetic disruption of a histamine-forming enzyme show impaired myeloid cell differentiation along with increased susceptibility to chemically induced cancer. HDC was also shown to enhance the expression of CD86 and HLA-DR on monocyte-derived dendritic cells (DCs) and to enhance the expression of CD11 b on myeloid cells in a NOX2-dependent manner

These previous findings incited us to assess 87 the dynamics of myeloid cell populations in blood during immunotherapy with HDC/IL-2. We report that subsets of myeloid cells and their expression of maturation markers are modulated during HDC-based immunotherapy, and that the dynamics of myeloid cells in blood may be associated with clinical outcome in AML.

### Patients, Materials and Methods

Patients, study design and objectives. Eighty-four adult AML patients (age 18-79) in first CR were enrolled in the Re:Mission trial (NCT01347996, registered at www.clinicaltrials.gov). Detailed patient characteristics are accounted for in previous publications. The Re:Mission trial was a single-armed multicenter phase IV study where all patients received ten consecutive 21-day cycles of HDC and low-dose IL-2 for 18 months or until relapse or death. The regimen was identical to that employed in a previous phase III trial. Patients were enrolled at a median of 46 days after the completion of consolidation and were followed-up for at least 24 months after the onset of immunotherapy. Three patients withdrew consent and were not included in any analysis. An additional fourteen patients discontinued prematurely from the study and were censored at the last captured follow-up date. Relapse was defined as at least 5% blast cells in bone marrow or presence of extramedullary leukemia. The primary trial endpoints included the quantitative and qualitative effects of HDC/IL-2 on T and NK cell populations in peripheral blood during treatment cycles. The present analyses of effects on myeloid populations and their impact on clinical outcome (leukemia free survival, LFS and overall survival, OS) were performed post-hoc. The study was conducted according to the principles outlined in the Declaration of Helsinki and was approved by the Ethics Committees of each participating institution. All patients gave written informed consent before enrollment.

Isolation of PBMCs, staining and flow cytometry. Peripheral blood was collected before and after the first and third HDC/IL-2 treatment cycles. PBMCs were isolated and cryopreserved at local sites and shipped on dry ice to the TIMM Laboratory, University of Gothenburg for analysis by two myeloid panels, one to determine the expression of activation markers by 118 myeloid cells and the other to determine expression level of H₂R on monocytes. After thawing, the cryopreserved samples aimed for the activation panel were pre-incubated in human FC block (BD Biosciences, Stockholm, Sweden). The samples aimed for the H₂R panel were stained with LIVE/DEAD fixable yellow stain (Life Technologies, Grand Island, N.Y., USA). Thereafter the samples were incubated for 30 min at 4° C. with a cocktail of surface marker antibodies in Brilliant stain buffer (BD Biosciences) (activation panel), or in PBS containing 0.5% BSA and 0.1% EDTA (H₂R panel). The following anti-human monoclonal antibodies were utilized: CD3-PerCPCy5.5 (HIT3A), CD19-PerCPCy5.5 (SJ25C1), CD16-Brilliant Violet 605 (3G8), HLA-DR-APCH7 (G46-6), CD14-PECy7 (MφP9), CD141-APC (1A4), HLA-ABC-FITC (G46-2,6), CD40-PE (5C3), CD86-Brilliant Violet 711 (FUN1), CD11b-Pacific Blue (ICRF44), CD33-PECy7 (P67.6) (all from BD Biosciences), CD56-PerCP eflour 710 (CMSSB) (eBioscience, San Diego, Calif., USA), CD1c-Brilliant Violet 421 (L161) (Biolegend, San Diego, Calif., USA), anti-histamine H2 receptor (polyclonal rabbit IgG) (MBL International, Woburn, Mass., USA), goat anti-rabbit-APC and CD14-Qdot655 or CD14-Pacific blue (both clone TüK4) (Life Technologies).

The samples were analyzed on a 4-laser BD LSRFortessa SORP flow cytometer (405, 488, 532, and 640 nm; BD Biosciences). Data analysis was performed by using the FlowJo software, version 7.6.5 or later (TreeStar, Ashland, Oreg.). Blood samples were available from 81 out of 84 patients. Phenotype analysis of myeloid cells was performed on PBMC using the above-referenced myeloid panels. Forty-nine patients were analyzed using the H₂R panel and 59 samples were analyzed using the activation marker panel. These patients were selected based on the availability of viably of frozen PBMC. Differential counts were performed by the participating centers on whole blood and were available for the 81 patients who consented to continue on study. The differential counts were use 143 d to determine the absolute counts of myeloid populations (eosinophils, neutrophils and monocytes) in blood.

Statistical analyses. Comparisons of blood cell counts and expression of maturation markers on myeloid cells before and after immunotherapy were performed using Prism 6 by Student's paired t-test, as specified in the statistical study plan. All reported significances using Student's paired t-test remained significant also by Wilcoxon matched pairs test. The impact of these immune parameters on LFS and OS at the trial closing date (Oct. 13, 2014) was determined using the logrank test. Parameters that significantly predicted LFS or OS with the logrank test were further analyzed by Cox univariable and multivariable regression analyses using the SPSS statistics 24 software. In multivariable analyses, continuous parameters such as fluorescence intensity were converted to nominal values based on above or below median expression. Univariable Cox analysis was utilized to determine the impact of age, risk group (classified according to recommendations by the European LeukemiaNet (14)), number of induction courses required to achieve CR (1 or >1) and number of consolidation courses (0-2 or >2) on LFS and OS. Prognostic factors with a P-value below 0.1 for LFS (age and number of induction cycles) were included as potential confounders in the multivariable Cox analysis.

### Results

Immunotherapy with HDC/IL-2 increases eosinophil counts in blood Eighty-four adult patients with AML in first CR participated in the Re:Mission trial. The patients received 10 consecutive 3-week cycles of immunotherapy with HDC/IL-2 in the post-consolidation phase. Peripheral blood was collected before and after treatment cycles 1 and 3 and analyzed for content of leukocyte populations by differential counts. In addition, PBMC isolated from blood was analyzed by flow cytometry for content of monocyte and DC populations and their expression of activation markers.

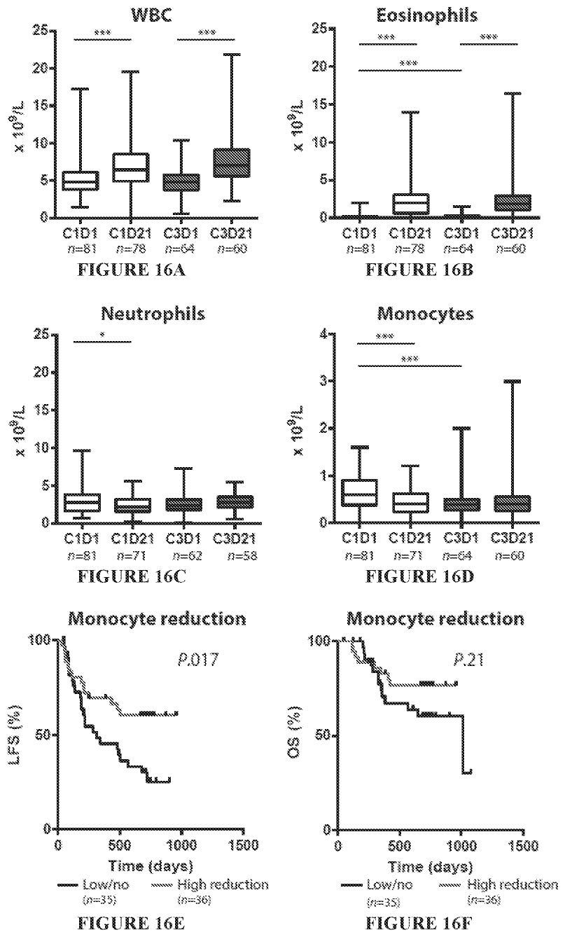

During treatment cycles, there was a marked increase in the number of total white blood cells that was normalized between cycles (FIG. 16A). The induction of white blood cells was mainly contributed by eosinophils whose numbers typically increased ten-fold during treatment cycles (FIG. 16B). Although the eosinophil counts contracted between treatment cycles, the eosinophil counts at the start of cycle 3 remained almost twice as high as before the start of cycle 1 (FIG. 16B). Neither the eosinophil counts before treatment cycles, after treatment cycles, nor the induction of eosinophils during treatment cycles impacted significantly on clinical outcome in terms of LFS or OS (data not shown).

Treatment with HDC/IL-2 reduces monocyte counts in blood. In contrast to the increment of eosinophil counts during HDC/IL-2 therapy, the number of monocytes and neutrophils was reduced during the first treatment cycle (FIG. 16C-D). There was no further reduction of monocyte or neutrophil counts during cycle 3. The monocyte counts remained reduced in cycle 3 as compared with levels at the onset of immunotherapy (FIG. 16D).

Neutrophil counts in blood before or after immunotherapy 193 did not impact on clinical outcome (data not shown). However, when patients were dichotomized by the median into high or low/no reduction in monocyte counts during the first treatment cycle, a strong monocyte reduction significantly predicted LFS (FIG. 16E) with a similar trend for OS (FIG. 16F). The impact of monocyte reduction on LFS remained significant in univariable but not multivariable regression analysis (Table 5).

TABLE 5
Univariable and multivariable Cox regression analyses of the impact
of reduction of monocyte counts, monocyte expression of H₂R and
monocyte expression of HLA-ABC on LFS and OS.

| | Univariable analysis | | | Multivariable analysis¹ | | |
|---|---|---|---|---|---|---|
| | Hazard | Confidence | p- | Hazard | Confidence | p- |
| Variable | ratio | interval | value | ratio | interval | value |
| Monocyte reduction during C1 vs LFS | 2.19 | 1.13-4.25 | 0.02 | 1.77 | 0.88-3.56 | 0.110 |
| CD14⁺⁺ H₂R expression C1D21 vs LFS | 2.98 | 1.36-6.50 | 0.006 | 2.82 | 1.27-6.28 | 0.011 |
| CD16⁺ H₂R expression C1D21 vs LFS | 3.11 | 1.43-6.76 | 0.004 | 3.13 | 1.43-6.87 | 0.004 |
| CD14⁺⁺ H₂R expression C1D21 vs OS | 3.71 | 1.18-11.70 | 0.025 | 3.40 | 1.07-10.82 | 0.038 |
| CD16⁺ H₂R expression C1D21 vs OS | 5.7 | 1.61-19.93 | 0.007 | 6.17 | 1.73-22.09 | 0.005 |
| CD14⁺⁺ HLA-ABC expression C1D1 vs LFS | 0.36 | 0.18-0.72 | 0.004 | 0.34 | 0.17-0.71 | 0.004 |
| CD14⁺⁺ HLA-ABC expression C1D1 vs LFS² | 0.20 | 0.06-0.66 | 0.008 | 0.32 | 0.09-1.20 | 0.092 |
| CD14⁺⁺ HLA-ABC expression C1D21 vs | 0.27 | 0.107-0.70 | 0.007 | 0.320 | 0.12-0.84 | 0.021 |
| LFS³ | | | | | | |

¹Multivariate analyses were corrected for age and number of induction cycles.
²Patients with below median frequency of CD8⁺ T_{EM} C1D1 were included in the analysis.
³Patients not displaying CD8⁺ T_{EM} to T_{eff} cell transition during cycle 1 were included in the analysis.

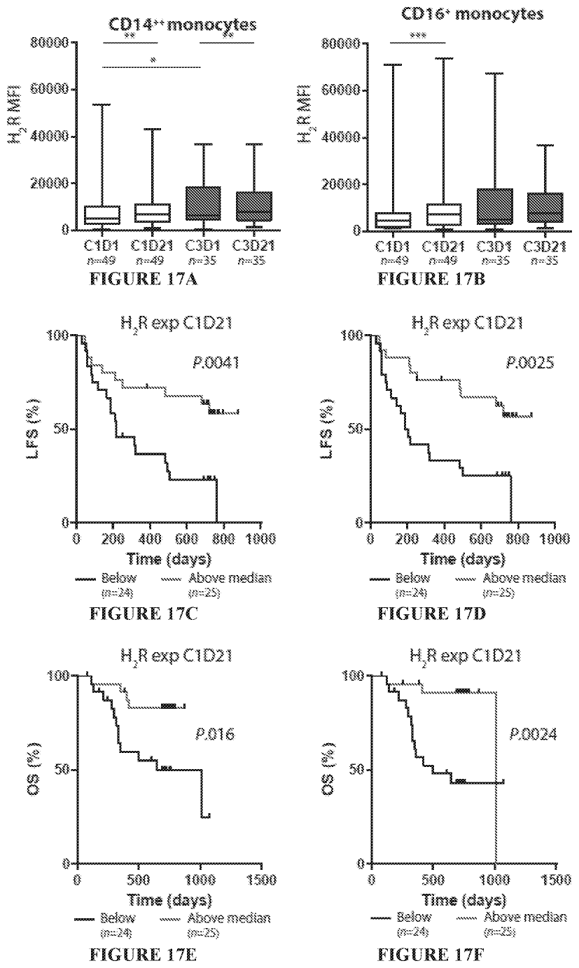

Monocyte expression of H₂R s predicts relapse risk and survival in patients undergoing HDC-based immunotherapy. The two major monocyte populations in blood, CD14⁺⁺CD16⁻(CD14⁺⁺) and CD14⁺CD16⁺(CD16⁺) cells decreased to a similar extent during treatment with HDC/IL-2 (data not shown). Earlier studies show that HDC ligates histamine type 2 receptors (H₂Rs) to inhibit NOX2-derived ROS formation in vitro in addition to promoting the differentiation of myeloid cells. The expression of H₂R on CD14⁺⁺ and CD16⁺ monocytes was assessed during immunotherapy and correlated to clinical outcome. As shown in FIG. 17A-B, the expression of H₂Rs was significantly enhanced on CD14⁺⁺ monocytes during and between treatment cycles, and on CD16+ monocytes during the first HDC/IL-2 treatment cycle. A high H₂R expression on CD14++ or CD16⁺ monocytes after the first treatment cycle strongly prognosticated LFS and OS (FIG. 17C-F). Similar trends towards a beneficial impact of high monocyte H₂R expression were observed at onset of immunotherapy (data not shown). The impact of monocyte H₂R expression on LFS and OS remained significant in multivariable analysis (Table 5).

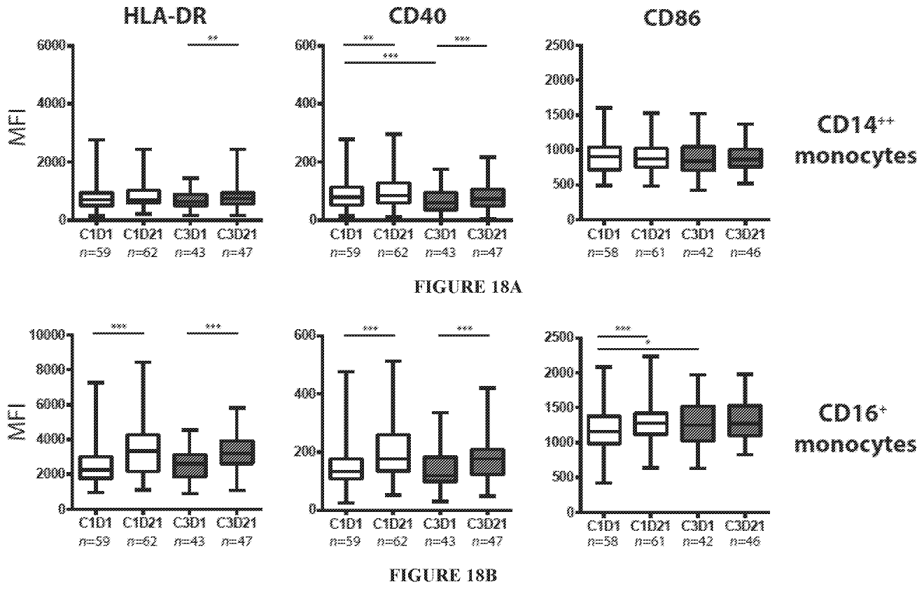

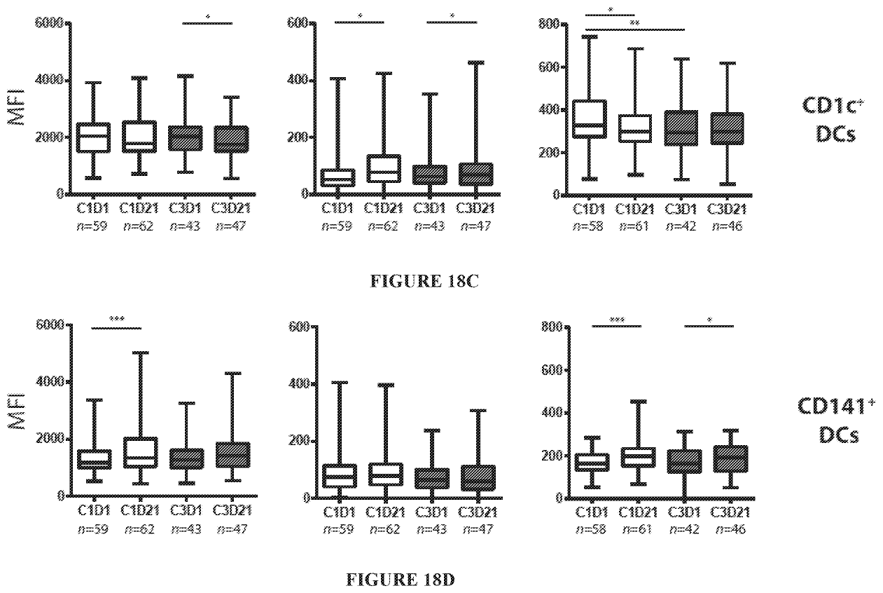

Treatment with HDC/IL-2 increases the expression of activation markers on monocytes and DCs. To further clarify effects of HDC/IL-2 treatment on cells of myeloid origin, the expression of the activation markers HLA-DR, CD40 and CD86 was determined before and after treatment cycles 1 and 3. The results showed increased expression of these activation markers on both CD14⁺⁺ and CD16⁺ monocytes during treatment cycles (FIG. 18A-B). For CD14⁺⁺ monocytes, a significant increase in HLA-DR expression was observed during cycle 3 and in CD40 expression during both treatment cycles (FIG. 18A). For CD16⁺ monocytes, all analyzed activation markers increased significantly during at least one of the HDC/IL-2 treatment cycles (FIG. 18B).

The two major DC populations in blood carry CD1c⁺ and CD141⁺ DCs phenotypes, respectively. Similar to monocytes, the numbers of DCs were reduced during HDC/IL-2 immunotherapy (data not shown). Regarding expression of DC-related activation markers, there were discrepancies between CD1c⁺ and CD141⁺ DC populations. The expression of CD40 increased during treatment with HDC/IL-2 only on CD1c⁺ DCs whereas the expression of HLA-DR and CD86 was induced only on CD141⁺ DCs (FIG. 18C-D). The expression of HLA-DR and CD86 was, in contrast, slightly decreased on CD1c⁺ DCs during HDC/IL-2 treatment (FIG. 18C). No significant correlation was observed between clinical outcome and the expression of HLA-DR, CD40 or CD86 on monocytes or DCs (data not shown).

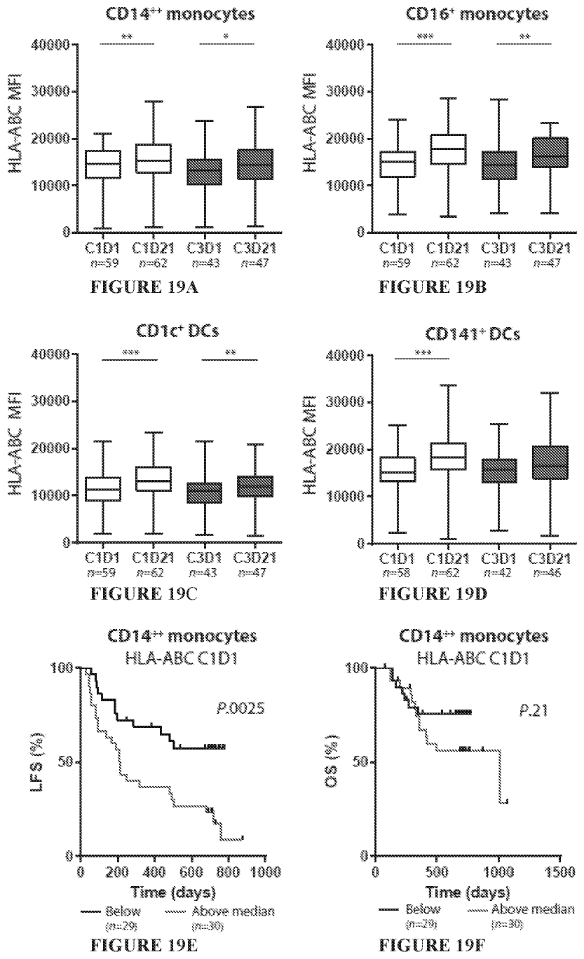

Low HLA-ABC expression on myeloid cells heralds favorable clinical outcome in patients with altered distribution of cytotoxic T cells in blood. Treatment with HDC/IL-2 consistently entailed enhanced expression of HLA-ABC during treatment cycles on all monocyte and DC populations analyzed (FIG. 19A-D). A high expression of HLA-ABC on CD14⁺⁺ monocytes at onset of immunotherapy heralded poor prognosis (FIG. 19E-F), which remained significant in multivariable analysis (Table 1). A similar trend towards short LFS for patients with high HLA-ABC expression on CD14⁺⁺ monocytes was observed after the first HDC/IL-2 treatment cycle (P=0.15, n=62, logrank test). The expression of HLA-ABC on CD16⁺ monocytes, CD1c⁺ DCs and CD141⁺ DCs were highly correlated to HLA-ABC expression of CD14⁺⁺ monocytes (data not shown). A high pre-treatment expression of HLA-ABC on these myeloid populations also predicted poor clinical outcome (data not shown).

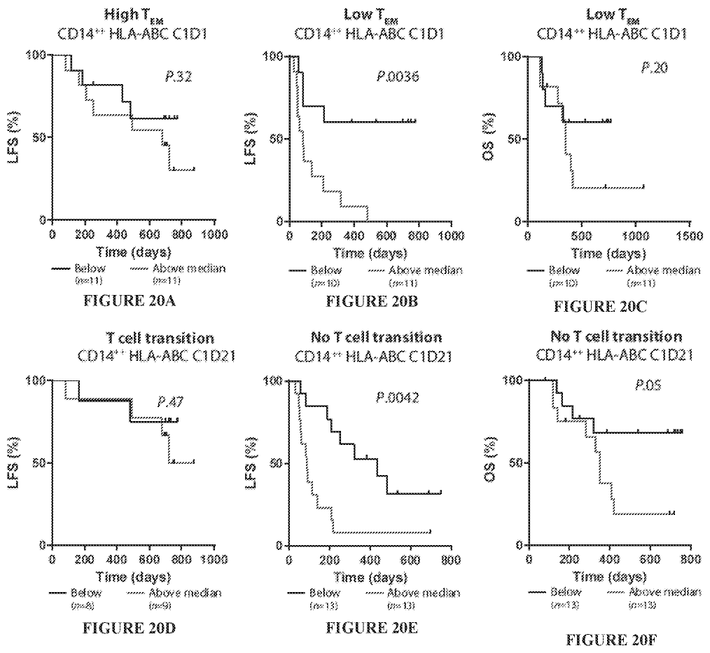

We speculated that the HLA-ABC expression on residual myeloid malignant cells may be similar to the HLA-ABC expression on monocytes and DCs. A high HLA-ABC expression on malignant cells would thus facilitate the interaction with cytotoxic T cells to recognize and destroy malignant cells, but also hinder activation of NK cell cytotoxicity by interaction with killer-cell immunoglobulin-like receptors (KIR). In a previous report from the Re: Mission trial, we observed that patients with a high frequency of CD8⁺ effector memory cells (T_{EM}; CD45RO⁺CCR7⁻) in blood at the start of immunotherapy, or patients undergoing a transition of CD8⁺ T_{EM} cells to CD8⁺ T effector cells (T_{Eff}; CD45RA⁺CCR7⁻) during the first HDC/IL-2 treatment cycle, showed improved LFS and OS. These factors may be indicative of a functional T cell immunity of relevance to reduced relapse risk. Among patients with above median frequency of CD8⁺ T_{EM} cells at onset of therapy, the expression of HLA-ABC on CD14⁺⁺ monocytes C1D1 did not impact on relapse risk (FIG. 20A). In contrast, the favorable impact of low monocytic HLA-ABC expression was pronounced in patients harboring few CD8⁺ TEM cells at onset immunotherapy (FIG. 20B-C, Table 1). Similarly, the patients in whom T_{EM} to T_{Eff} transition was observed showed no apparent benefit of a low HLA-ABC expression on monocytes (FIG. 20D), while transition-negative patients benefited significantly form having a low monocytic HLA-ABC expression on Cl D21 (FIG. 20E-F, Table 5).

### Discussion

Leukemic relapses in the post-chemotherapy phase of AML are assumed to result from the expansion of residual malignant cells, and the goal of post-consolidation immunotherapy is to eradicate these remaining cells. Although cytotoxic T cells and NK cells are considered the main effector populations for surveillance of leukemic cells, proper functioning of the myeloid immune cells is critical for the activity of these lymphocytes. In HDC/IL-2 combination immunotherapy, the proposed function of the IL-2 component is to activate cytotoxic lymphocytes, while the HDC component targets myeloid-induced immunosuppression by inhibiting the production of immunosuppressive ROS and by promoting maturation of myeloid cells.

The pronounced eosinophilia observed during treatment cycles with HDC/IL-2 is in accordance with previous AML trials in which monotherapy with IL-2 was shown to trigger eosinophilia, probably as the result of IL-2-induced production of IL-5 and GM-CSF. Hence, the induction of eosinophils observed during treatment with HDC/IL-2 is likely attributed to the IL-2 component. Notably, the dose of IL-2 used in our study was lower than in most previous studies, and a contribution by the HDC component to the level of eosinophilia cannot be formally excluded. We observed no correlation between eosinophil counts and clinical outcome in terms of relapse risk or survival.

In addition to eosinophilia, the administration of IL-2 has been reported to cause neutropenia in AML patients. In our study, using low doses of IL-2 combined with HDC, a mild and transient reduction of blood neutrophils was observed during the first treatment cycle. A similar and more pronounced reduction of monocyte counts was observed during the first cycle of HDC/IL-2. Monocyte counts, but not neutrophil counts, remained at a lower level at the start of treatment cycle 3. Phenotype analyses revealed that the blood counts of both CD14⁺⁺ and CD16⁺ monocytes were reduced during HDC/IL-2 therapy. While information regarding effects of HDC or IL-2 on monocyte counts from previous AML trials is scarce, results from clinical trials in patients with metastatic renal cell carcinoma (RCC) indicated that IL-2 increases rather than reduces monocyte counts in blood. Interestingly, in an RCC trial by Donskov et al. the addition of HDC to the IL-2 regimen prevented monocytosis. In further support of HDC as an inhibitor of immature myeloid cells, genetic deficiency of histamine synthesis in mice is associated with a pronounced accumulation of immature myeloid cells. We thus hypothesize that the HDC component contributed to the observed reduction in monocyte counts in our HDC/IL-2 AML trial. Intriguingly, similar to neutrophils and monocytes, also the numbers of CD1c⁺ and CD141⁺ DC were reduced during immunotherapy in our trial. This was unexpected as the administration of HDC was previously shown to enhance DC numbers in tumor-bearing mice. However, in one of the above referred RCC trials, IL-2 treatment was found to reduce blood DC counts. Hence, we speculate that, in contrast to the reduction of monocytes, the reduction in DC numbers may be attributed to IL-2. It should, however, be noted that AML patients in CR have recently undergone intensive chemotherapy, and the observed fluctuations in leukocyte counts may, at least in part, reflect events occurring during recovery of bone marrow function.

In several forms of cancer, inappropriately activated monocytes and neutrophils are linked to immunosuppression and their presence in blood and other tissues often heralds poor prognosis for survival. In RCC, low monocyte and/or neutrophil counts before and after IL-2 treatment or a reduction in monocyte counts during IL-2 therapy have been shown to predict favorable clinical outcome. In the present study, a strong reduction of monocyte counts during the first HDC/IL-2 treatment cycle was associated with LFS, thus supporting the notion that presence of monocytes in blood may dampen the effectiveness of immunotherapy.

The proposed effects of HDC on myeloid cells include inhibition of NOX2-dependent ROS formation and promotion of myeloid cell maturation. These effects are strictly mediated via ligation of H₂R s expressed on myeloid cells. In the Re:Mission trial it was observed that the expression of H₂Rs was strongly enhanced on CD14⁺⁺ as well as on CD16⁺ monocytes during HDC/IL-2 therapy, in particular during the first treatment cycle. A high monocyte H₂R expression at C1D21 heralded improved LFS and OS, which remained significant in multivariable analysis taking potential confounders into account. We also observed a trend towards favorable survival of patients with a high density of H₂R s at the onset of immunotherapy. A high expression density of H₂Rs is likely to enhance the responsiveness of myeloid cells to HDC treatment, and might be considered as a biomarker of the efficiency of immunotherapy with HDC/IL-2. Further studies are warranted to determine the importance of myeloid cell H₂R expression for the clinical course of AML, including the potential impact of H₂R expression in patients not receiving immunotherapy.

In addition to H₂R, the expression of several other markers of maturation increased significantly on monocyte and DC populations during treatment cycles indicating that the immunotherapy triggers myeloid maturation of human cells not only in vitro but also in vivo. Unexpectedly, however, a high myeloid cell expression of HLA-DR, CD40 or CD86 did not impact on LFS, while a high expression of HLA-ABC was associated with enhanced risk of relapse. The monocyte expression of HLA-DR, CD86 and CD40 correlated strongly to the monocytic HLA-ABC expression before and after the first cycle of immunotherapy (data not shown). Similar correlations were found for DC expression of HLA-DR and HLA-ABC before and after treatment with HDC/IL-2 (data not shown). We thus speculate that the lack of positive impact of HLA-DR, CD40 or CD86 expression may relate to the correlation of these maturation markers with the negative prognostic marker HLA-ABC.

Several lines of evidence imply that NK cell and T cell functions are important for a favorable clinical outcome of AML. During HDC/IL-2 immunotherapy it was previously reported that NK cell-related parameters, including NK cell counts and NK cell expression of natural cytotoxicity receptors (NCR) significantly predict relapse and death in the post consolidation phase. In addition, parameters related to functional cytotoxic (CD8⁺) T cells, including high numbers of CD8⁺ T_{EM} cells at onset of therapy, CD8⁺ T cell transition from T_{EM} to T_{eff} cells and presence of CD8⁺ T cells that are reactive with AML-derived antigens, prognosticate a favorable clinical outcome. Our previous results also suggest that NK cells and CD8⁺ T cells may constitute independent effector arms in the prevention of relapse of AML.

In the present study, a high expression of HLA-ABC on myeloid cells was associated with poor prognosis, and may hence be a conceivable biomarker. The strong correlation between the HLA-ABC expression on monocytes and all other analyzed myeloid cell populations suggests that monocyte HLA-ABC expression may reflect the HLA expression also on residual leukemic blasts. HLA-ABC expressed on leukemic blasts may inhibit NK cell cytotoxicity via ligation of KIRs expressed by the NK cells. Interestingly, the favorable impact of low HLA-ABC expression on LFS was apparently restricted to patients displaying unfavorable distribution of CD8⁺ T cells, i.e. a low frequency of CD8⁺ T_{EM} cells and lack of T_{EM} to T_{Eff} transition. Patients with an unfavorable T cell distribution may rely on NK cellmediated surveillance of leukemic cells, which thus provides a possible explanation to the favorable clinical impact of low HLA-ABC expression in this group of patients. The preliminary nature of these conclusions must be emphasized, and further studies are warranted on the possible interplay between myeloid cell HLA-ABC expression and the anti-leukemic function of NK cells and T cells in AML.

We conclude that the phenotypes and dynamics of myeloid cell populations may be relevant to relapse risk in AML, and that strategies to target aspects of myeloid cell function may ameliorate the efficiency of immunotherapies aiming to prevent relapse.

### Example 7

### NOX2-Dependent Immunosuppression in Chronic Myelomonocytic Leukemia

Chronic myelomonocytic leukemia (CMML) is a myeloproliferative and myelodysplastic neoplasm with few treatment options and dismal prognosis. The role of natural killer (NK) cells and other anti-leukemic lymphocytes in CMML is largely unknown. We aimed at providing insight into the mechanisms of immune evasion in CMML with focus on immunosuppressive reactive oxygen species (ROS) formed by the myeloid cell NADPH oxidase (NOX2). The dominant population of primary human CMML cells was found to express membrane-bound NOX2 and released ROS that in turn triggered extensive PARP 1-dependent cell death in co-cultured NK cells, CD8+T effector memory and CD8+T effector cells. Inhibitors of ROS formation and scavengers of extracellular ROS prevented CMML cell-induced lymphocyte death and facilitated NK cell degranulation towards antibody-coated primary CMML cells. In CMML patients, elevation of immature cell counts (CD34) in blood was associated with reduced expression of several NK cell-activating receptors. We propose that CMML cells may utilize extracellular ROS as a targetable mechanism of immune escape.

Patients with CMML present with elevated monocyte counts in blood (>1×10⁹/L) along with presence of blast cells in blood and bone marrow. CMML typically progresses slowly with signs of myeloproliferation and myelodysplasia. The median survival after diagnosis is <2 years with few long-term survivors, which is partly explained by a high risk of transition into acute myeloid leukemia (AML). The vast majority of CMML patients are >60 years old and ineligible for allogeneic bone marrow transplantation, which remains the only potentially curative treatment.

Reactive oxygen species (ROS) are produced by several types of myeloid cells as a defense strategy against microorganisms. However, ROS formed by the NADPH oxidase/NOX2 (NOX2) of monocytes and granulocytes have also been ascribed a role in immune regulation. Thus, intact capacity of myeloid cells to produce ROS via NOX2 is reportedly important to dampen the activity of autoreactive T cells. In cancer, the extracellular release of NOX2-derived ROS is associated with significant dysfunction of anti-neoplastic lymphocytes, in particular natural killer (NK) cells and subsets of T cells. An inhibitor of NOX2-mediated ROS formation, histamine dihydrochloride (HDC), is used in combination with the NK and T cell-activating cytokine interleukin-2 (IL-2) for relapse prevention in the post-chemotherapy phase of AML. Post hoc analyses of phase III trial results implied that the clinical efficacy of HDC/IL-2 is pronounced among patients with AML of monocytic differentiation where patients harbor leukemic cells that co-express a functional NOX2 and histamine type 2 receptors (H₂R).

For this study, we (1) determined the expression of NOX2 and H₂Rs on primary human CMML cells, (2) assessed the potential immunosuppressive properties of CMML cells and (3) determined phenotypes of circulating NK cells and CD8+ T cell subsets from CMML patients. Our results suggest that NOX2-derived ROS may constitute a targetable mechanism of leukemia-related immunosuppression in CMM.

Patients and healthy blood donors. Blood samples from patients with CMML seen at hospitals in Region Vastra Gotaland and Region Skane, Sweden, were obtained after written informed consent. The patients fulfilled the criteria for CMML according to the 2008 WHO classification. Out of twelve patients, 10 (83%) had CMML-1 (defined as <5% blasts, including promonocytes, in blood or <10% in bone marrow) whereas 2 (17%) had CMML-2. Ten patients were male and 2 were female (age 52-88 years, median 70). Absolute counts of circulating monocytes in CMML patients were available from 9 patients and ranged from 4.5×10⁹/L to 29.6×10⁹/L with a median of 10.0×10⁹/L. Leukopacks (buffy coats) from healthy blood donors were obtained from the Blood Centers at Sahlgrenska University Hospital and Kungalv Hospital, Sweden. For immunophenotyping of patient NK cells, control samples from age-matched individuals were used (age 50-82 years, median 65.5).

Isolation of cells. Peripheral blood mononuclear cells (PBMCs) from the patients and the healthy donors were isolated as described. In brief, blood samples or buffy coat leukocytes were subjected to density gradient centrifugation following dextran sedimentation. NK cells, CD8+ cells and monocytes from healthy donors were obtained by immunomagnetic isolation using the MACS NK cell isolation kit, CD8+ isolation kit and the MACS monocyte isolation kit II (Miltenyi Biotec, Stockholm, Sweden), respectively. Primary leukemic blasts and monocytes were isolated from patient PBMCs by FACS. Blasts were defined as CD14⁻CD34+ and monocytes as CD14+CD33+.

ROS production. The extracellular ROS production of primary CMML cells was measured by isoluminol-enhanced chemiluminescence as described. In brief, 2×10⁵ cells were added to 96-well plates in the presence of isoluminol (10 μg/ml) and horseradish peroxidase (HRP; 4U/ml) and stimulated by the NOX2 inducer N-formyl-methionyl-leucyl-phenylalanine (fMLF; 0.1 μM). Light emission was recorded using a FLUOstar Omega platereader (BMG Labtech).

Lymphocyte cell death. Isolated lymphocyte subsets were co-cultured overnight with primary leukemic monocytes from CMML patients with or without NOX2 inhibitors or other anti-oxidative compounds at 37° C. in 5% CO₂. Lymphocyte cell death was assessed by flow cytometry after staining with a LIVE/DEAD® fixable dead cell stain (Life Technologies). In receptor expression assays, purified NK cells where co-cultured overnight with monocytes in the presence or absence of the PARP-1 inhibitor PJ34 (0.504). The following antibodies were used for receptor expression assessment: NKp46-PE (clone: 9E2) and DNAM-1-PE-Cy7 (clone: DX11) from Miltenyi Biotec. NKp80-APC (clone: 4A4.D10) and CD16-BV605 (clone: 3G8) from BD Pharmingen.

Subsets of CD8+ T cells were stained and sorted on a 3-laser FACSARIA II flow cytometer (BD Biosciences). The following antibodies were used to distinguish the T cell populations: CD3-Pacific Blue (clone: S4.1) from Life technologies. CD8-APC-H7 (clone: SK1), CD45RA-APC (clone: HI100), CD45RO-PE (clone: UCHL1) and CCR7-PE-Cy7 (clone: G043H7) all from BD Pharmingen. CD8+ T cell subsets were further defined as follows: naive T cells (TN) as CD45RA CCR7+, T central memory cells (T_{CM}) as CD45ROCCR7+, T effector memory cells (T_{EM}) as CD45ROCCR7⁻ and T effector cells (T_{Eff}) as CD45RACCR7⁻.

Assays of NK cell degranulation and ADCC. NK cell degranulation and cytotoxicity against patient-derived, malignant monocytes were assessed in experiments using lintuzumab (Abbvie, Stockholm, Sweden), a humanized anti-CD33 antibody. NK cells from healthy donors were co-incubated with monocytes, labeled with CellTrace violet stain (Life Technologies), in the presence of anti-CD107a-PE-Cy7 antibody (BD Pharmingen) and anti-oxidative compounds for four hours at 37° C. and 5% CO₂. Thereafter, the cells were stained with LIVE/DEAD® fixable dead cell stain (Life Technologies) and assessed for lysis of monocytes and NK cell degranulation by flow cytometry. For culture experiments, cytotoxicity assays and confocal microscopy, Iscoves' modified Dulbecco minimum essential medium (IDMEM) supplemented with 10% human AB+ serum was used.

Confocal microscopy. Primary leukemic monocytes from CMML patients were stained with anti-flavocytochrome b558 (gp91^{phox}) (clone 7D5, FITC) from Novus Biologicals and subsequently incubated on a confocal slide for 45 min at a ratio 1:2 with healthy NK cells in the presence of lintuzumab (1 μg/ml). Cells were then washed extensively in PBS and mounted with ProLong Gold antifade reagent with DAPI (Invitrogen). Photomicrographs were obtained using a Zeiss LSM700 confocal microscope and analyzed using ZEN Blue software from Zeiss.

Compounds. The following compounds were used: histamine dihydrochloride (HDC; 100 μM; Sigma-Aldrich), the H₂R receptor antagonist ranitidine (Glaxo, Molndal, Sweden), AH202399AA (a chemical control to ranitidine, Glaxo), catalase (200U/ml; Sigma-Aldrich), interleukin-2 (IL-2; 500U/ml; Chiron), the NOX2 inhibitor diphenylene iodonium chloride (DPI; 304; Sigma-Aldrich), and the PARP-1 inhibitor PJ34 (0.5 μM; Sigma-Aldrich).

Immunophenotyping by flow cytometry. NK cell expression of activating receptors before and after cell culture was measured by flow cytometry in 10 CMML patients and 10 healthy age-matched controls. In these experiments, cells were cultured in 96-well plates for 3 days at a concentration of 1×10⁶ cells per well in 200 μl with IL-2 (500 u/ml) present. Cells were then stained on days 1 and day 3 and analyzed using a 4-laser BD LSRFortessa SORP flow cytometer. NK cells were defined as CD37CD56+ cells. The analyses comprised the following receptors of relevance to NK cell activation or maturation: NKp30-PE (clone: AF29-4D12), NKp80-APC (clone: 4A4.D10) and DNAM-1-PE-Cy7 (clone: DX11), all from Miltenyi Biotec. NKp46-PE (clone: 9E2), NKG2D-PE-Cy7 (clone: 1D11), LFA-1b-APC (clone: 6.7), CD57-FITC (clone: HNK-1) were from BD Pharmingen. 2B4-FITC were from R&D systems and NKG2C-Pacific Blue (clone: MM0488-9R17) from Novus Biologicals.

Monocytes and blasts from CMML patients were analyzed using a panel consisting of anti-CD33 (clone P67.6, PE-Cy7), anti-CD34 (clone 8G12, PE), anti-CD56 (clone NCAM16, PE-Cy7, APC), anti-CD14 (clone MϕP9, APC-Cy7), anti-CD15 (clone HI98, APC), all from BD Pharmingen. Anti-flavocytochrome b558 (gp91^{phox}) (clone 7D5, FITC). Anti-histamine H₂ receptor (polyclonal rabbit IgG) was from MBL International (Woburn, Mass.) and goat anti-rabbit secondary antibody (PE-Cy5.5) was from Invitrogen.

Statistical analysis and ethical considerations. One-way ANOVA followed by Bonferroni's post-test was used for multiple comparisons and the paired t-test for single comparisons. P-values<0.05 were considered significant. In figures, asterisks are used as follows: *P<0.05, **P<0.01, ***P<0.001. All indicated P-values are two-sided. The study was approved by the Ethical Review board of Gothenburg and all experiments were performed in accordance with the Declaration of Helsinki.

### Results

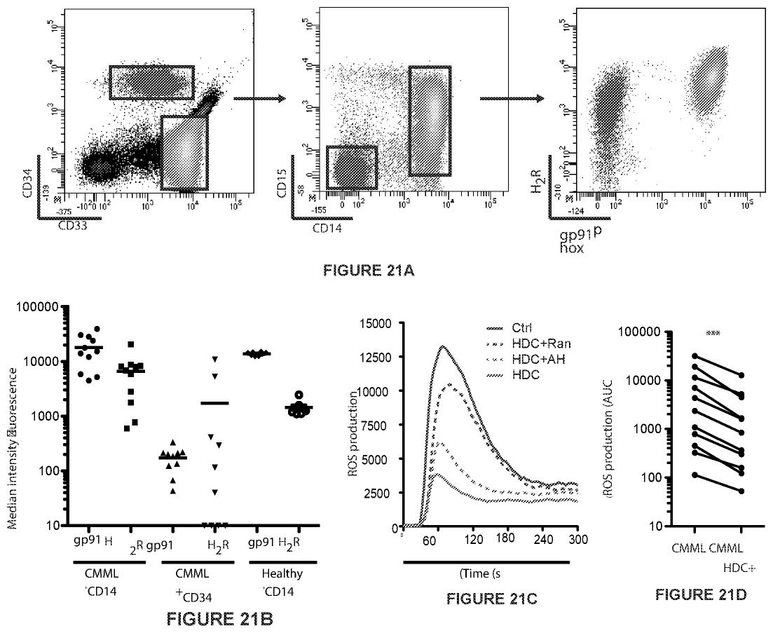

Monocytic CMML cells express functional NOX2 and histamine H₂ receptors. Immunosuppressive ROS are produced by malignant granulocytes in chronic myeloid leukemia and by malignant monocytic cells in AML but the potential ROS-forming capacity of primary CMML cells has remained unknown. In initial experiments, we analyzed the production of ROS from human CMML cells along with their expression of NOX2, as reflected by the median fluorescence intensity (MFI) of the cell membrane-bound NOX2-specific subunit gp91^{phox}. The monocytic CD14+ population in peripheral blood from CMML patients expressed similar levels of gp91^{phox} as did CD14+ monocytes from healthy donors (FIGS. 21A and B). The fraction of CD14+ monocytes positive for gp91^{phox} was in excess of 99% for all CMML patients (data not shown).

Signaling via H₂-receptors (H₂R) has been shown to transduce histamine-induced inhibition of ROS formation. The primary monocytic CMML cells invariably expressed high levels of H₂Rs, suggesting that NOX2 activity may be targeted by HDC (FIGS. 21A and B). While the majority of CMML cells were CD33CD14+, a smaller population of CD34+ blasts was detected in blood in most patients. In contrast to the more mature monocytic CMML cells, the CD34 blast cells expressed low levels of gp91^{phox} and moderate levels of H2Rs (FIGS. 21A and B). To determine whether the expression of gp91^{phox} reflected a functional NOX2, patient CD14+ cells were sorted by FACS followed by analysis of ROS production after stimulation with fMLF, a formylated tripeptide of bacterial origin that triggers ROS formation in human myeloid cells. The sorted monocytic CMML cells were found to produce high levels of ROS and the addition of HDC significantly suppressed ROS production (FIGS. 21C and D). The ROS-inhibitory effect of HDC was, in turn, antagonized by ranitidine, a specific H₂R antagonist but not by a chemical control to ranitidine, AH202399AA, in which a thioether group has been replaced by an ether thereby reducing its affinity at H₂R>50-fold (FIG. 21C). These results imply that HDC suppresses NOX2-mediated ROS production in CMML cells by targeting H2R.

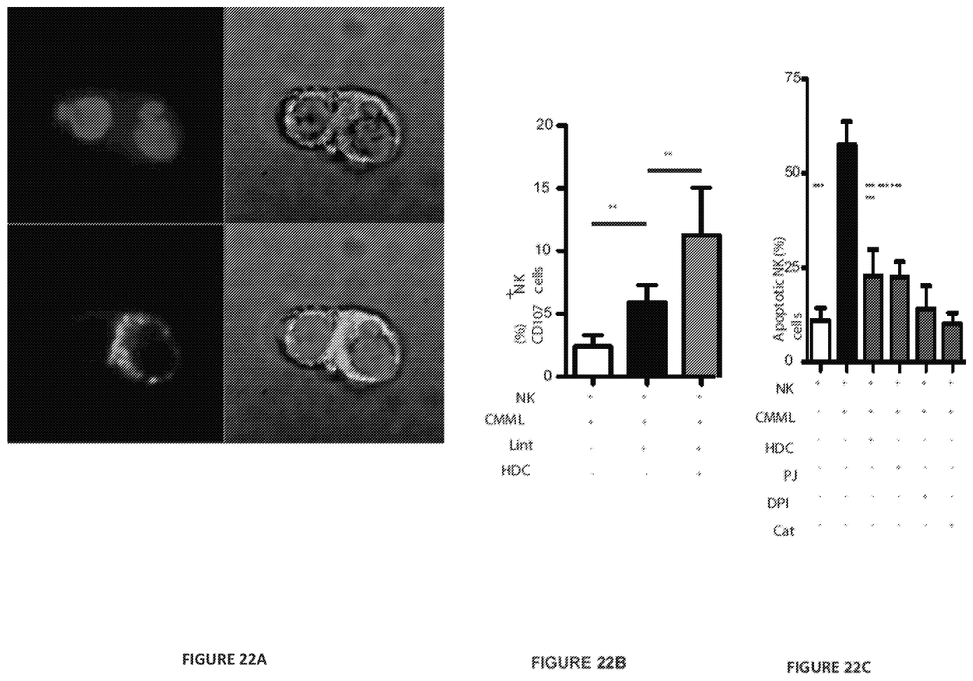

Inhibition of NOX2 promotes NK cell degranulation and ADCC towards antibody-coated primary CMML cells. Healthy and malignant myeloid cells were previously shown to compromise anti-tumor functions of NK cells by producing ROS [16]. We acquired confocal micrographs of interacting primary CD14+ CMML cells and NK cells in co-culture experiments. When analyzing the interaction between CD14+ CMML cells and NK cells we observed a distinct synapse between cells and a polarization of membrane-bound NOX2 towards the NK cell (FIG. 22A). The finding that monocytic CMML cells expressed NOX2 in the proximity of the immunological synapse along with their capacity to produced high levels of ROS incited us to study whether ROS production may constitute a mechanism by which CMML cells escape immune-mediated killing. To this end, we tested the ability of IL-2-stimulated NK cells to degranulate in response to primary CD14+ CMML cells in the presence or absence of lintuzumab (anti-CD33) as the linking antibody to enhance the interactions between NK cells and CMML cells. NK cells alone displayed little degranulation against primary CMML cells. The addition of lintuzumab significantly augmented NK cell responses (p<0.01, n=9), and the addition of the NOX2 inhibitor HDC further enhanced the degranulation response against the primary leukemic cells (p<0.01, n=9; FIG. 22B).

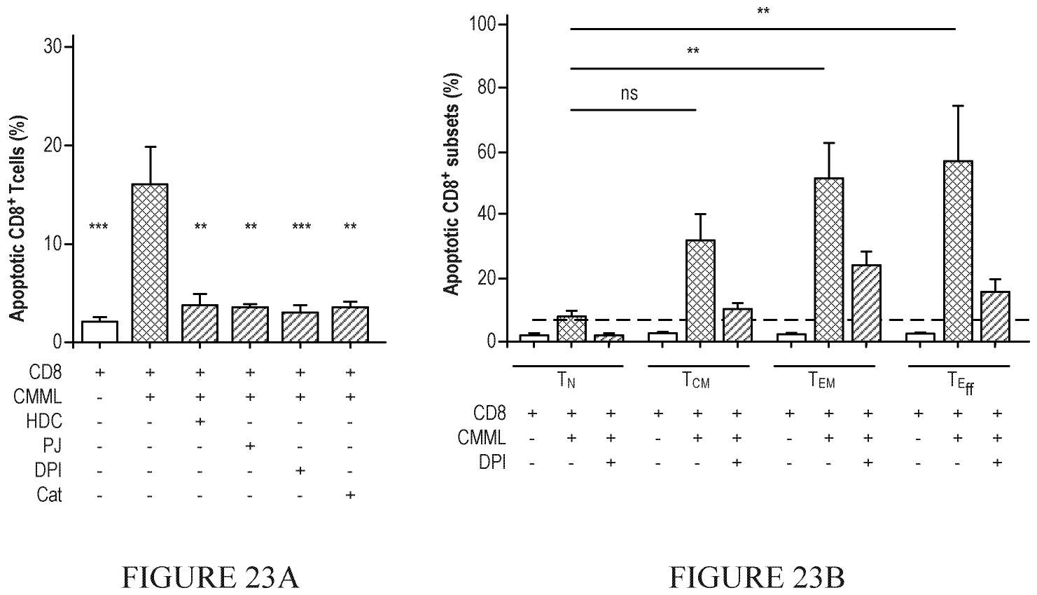

CD14+ CMML cells induce PARP 1-dependent cell death in lymphocytes. In overnight co-culture experiments, primary CD14+ CMML cells triggered significant cell death in healthy NK cells (p<0.001, n=6, FIG. 22C) and CD8+ T cells (p<0.001, n=4, FIG. 23A). The CMML-induced lymphocyte apoptosis was prevented by the NOX2 inhibitors HDC and DPI and by catalase, which scavenges extracellular ROS by degrading H₂O₂. Lymphocytes were also rescued from CMML cell-induced cell death by PJ34, an inhibitor of PARP-1, thus suggesting that ROS formed by CMML cells induce PARP 1-dependent cell death, also known as parthanatos, in NK cells and CD8+ T cells (FIGS. 22C and 23A).

CMML cells are preferentially suppressive towards mature T cell subsets. We have previously shown that presence of CD8+T effector memory cells (T_{EM}) is associated with favorable prognosis of AML patients receiving immunotherapy with HDC/IL-2 for relapse prevention in the post-consolidation phase. To determine whether CMML cells specifically inhibit certain T cell subsets, we FACS-sorted CD8+ T cell subsets (T_{N}, T_{CM}, T_{EM} and T_{eff}) and cultured these subpopulations overnight with primary monocytic CMML cells. The distribution of CD8+ T cell subsets in blood was similar in CMML patients and in age-matched healthy control subjects (data not shown). It was observed that the CMML cells predominantly induced cell death in more mature CD8+ T cell subsets, i.e. T_{EM} and T_{eff} cells, while T_{N} cells were largely resistant to CMML cell-induced apoptosis (FIG. 23B). The difference in T cell subset apoptosis was predominantly explained by difference in ROS sensitivity as the NOX2 inhibitor DPI efficiently rescued all T cell subsets from CMML cell-induced apoptosis.

Deficiency of receptor expression in NK cells from CMML patients. Functions of NK cells are regulated by activating and inhibitory receptors expressed on the NK cell surface. Earlier studies show that a reduced expression of activating NK cell receptors in myeloid malignancies, including AML and MDS, heralds unfavorable prognosis for survival. Also, progression of MDS has previously been associated with down-modulation of activating NK cell receptors.

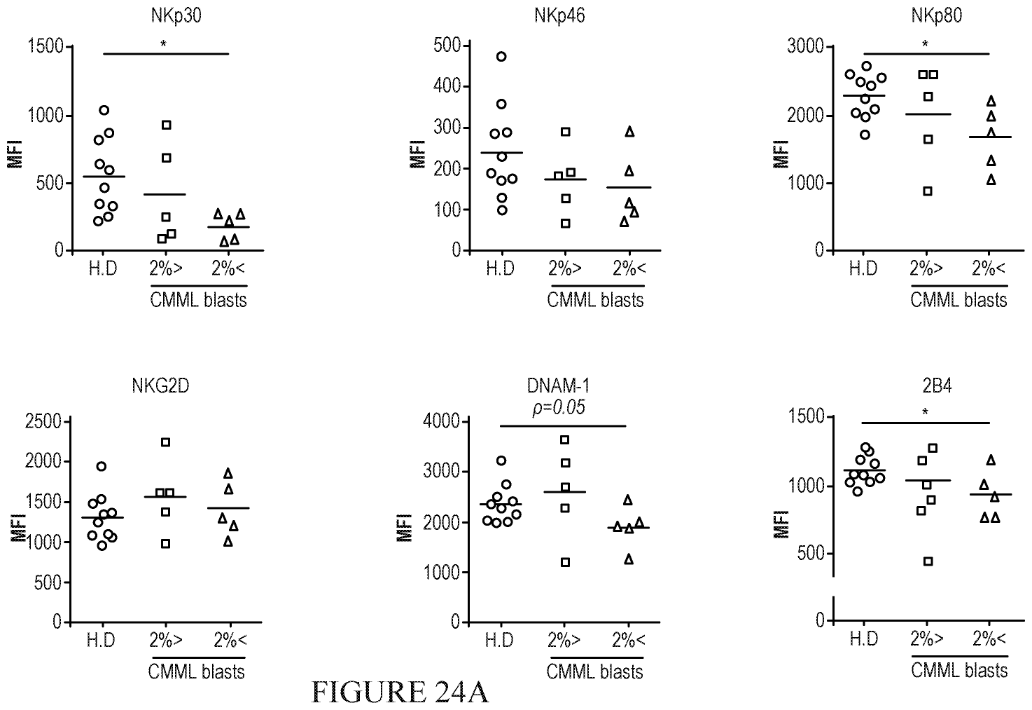

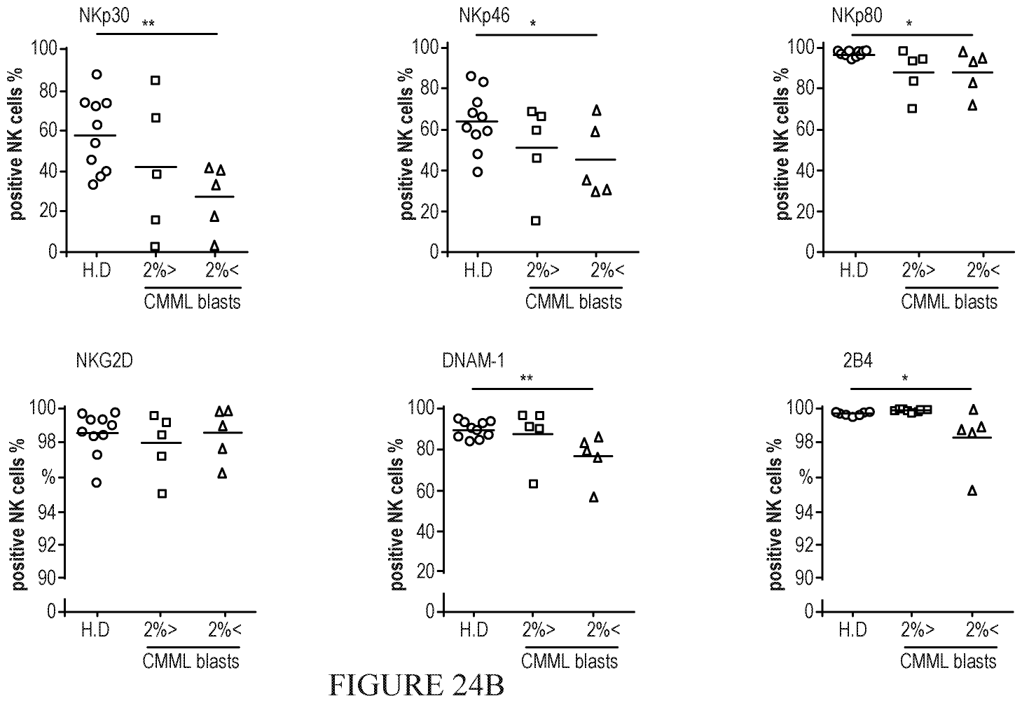

When analyzing patients with regard to the percentage of peripheral CD14⁻CD34+ blast cells, we found that the group of patients with a higher proportion of circulating CD34+ blasts (>2% of PBMC) showed significantly lower intensities of the activating NK cell receptors NKp30, NKp80 and 2B4 as compared with age-matched healthy controls (FIG. 24A). In addition, the frequency of circulating NK cells expressing NKp30, NKp46, NKp80, DNAM1 and 2B4 in blood was lower in CMML patients than in healthy controls (FIG. 24B). We also cultured PBMCs from patients or healthy donors in the presence or absence of IL-2 for 72h and determined the expression of NK cell receptors. It was observed that IL-2 stimulation significantly enhanced the expression of NKp30, NKp46 and DNAM1 in patient-derived NK cells (data not shown).

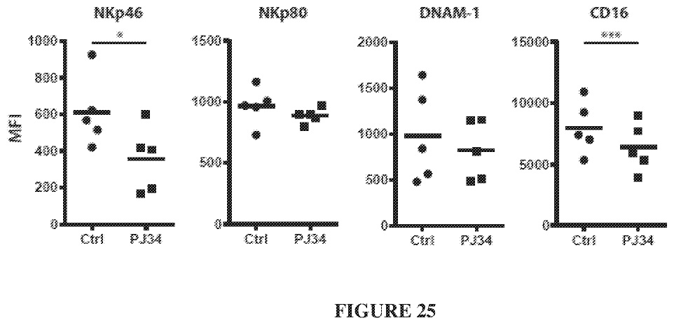

Earlier studies show that ROS-producing monocytes down-modulate the expression of activating NK cell receptors in vitro. It has remained unknown, however, whether the observed down-modulation of activating receptors reflects secondary events related to the initiation of NK cell apoptosis rather than a direct ROS-induced reduction of receptor expression by NK cells. We therefore exposed NK cells to ROS-producing monocytic cells from healthy donors in the presence of the PARP-1 inhibitor PJ34, which upholds the viability of NK cells despite the exposure to ROS. As shown in FIG. 25A, NK cells exposed to monocyte-derived ROS in the presence of PJ34 significantly downregulated NKp46 and CD16 with a similar trend for NKp80 and DNAM-1. No down-regulation of NK cell receptor expression was observed when NK cells were exposed to PJ34 in the absence of ROS-producing cells (data not shown).

### Discussion

NK cells and myeloid cells engage in bidirectional crosstalk to initiate immune responses, and NK cells have been proposed to shape, regulate and terminate immune responses by killing subsets of myeloid cells. NK cells also exert cytotoxicity against several malignant myeloid cells. A deficiency of NK cell function has been proposed to impact on the course of disease in myeloid leukemias. For example, low NK cell counts in blood are associated with relapse of leukemia after tyrosine kinase inhibitor discontinuation in patients with chronic myeloid leukemia and poor NK cell function and deficient expression of activating NCR herald dismal prognosis in patients with AML.

While details regarding the mechanisms underlying the NK cell deficiency in myeloid malignancies remain to be defined, the NOX2-dependent formation of extracellular ROS from myeloid cells has been proposed as a contributing pathway of immunosuppression. A main finding in this study was that CMML patients harbor monocytic leukemic cells that express functional NOX2 and that the release of NOX2-derived ROS from leukemic cells triggered dysfunction and cell death in lymphocytes with purported anti-leukemic function, including NK cells and CD8+ T cells. We also show that strategies to target NOX2-derived ROS, produced by CMML cells, maintained the viability of NK cells and subsets of CD8+ lymphocytes and upheld anti-leukemic functions of NK cells. These strategies included degradation of extracellular ROS (catalase) and specific inhibition of NOX2 (DPI) along with HDC that ligates H₂R to inhibit NOX2 function.

Relatively few studies have addressed the immunobiology of CMML or the role of anti-leukemic lymphocytes for the course of disease. In a cohort of 41 MDS patients (6 with CMML) Carlsten and coworkers observed that NK cells derived from bone marrow samples displayed reduced cytotoxicity compared with age-matched healthy controls. These authors also demonstrated a decreased surface expression of activating receptors (DNAM-1 and NKG2D) in patient-derived NK cells, which correlated with elevated bone marrow blast counts. While these previous studies did not allow separate analysis of the NK cell compartment in CMML, the results are compatible with our finding of a reduced expression of several activating receptors by NK cells from CMML patients, in particular in patients with higher counts of CD14⁻CD34+ blasts in blood. In accordance with previous studies we observed that ROS produced by monocytic cells induced downregulation of activating NK cell receptors. Experiments using the PARP-1 inhibitor PJ34 implied that ROS triggered downregulation of NK cell receptors also in the absence of cell death, thus supporting that ROS, rather than cellular events associated with early apoptosis, reduce the expression of activating NK cell receptors expression. Our results also imply that the NK cell receptor deficiency is at least partly reversible by cytokine activation (data not shown), the expression of NKp46, NKp30 and DNAM-1 on NK cells from CMML patients was restored by IL-2 to MFI levels comparable to those of healthy age-matched donors.

The role of cytotoxic T cells in CMML has not been characterized in detail. Allogeneic stem cell transplantation (allo-SCT) is the only potentially curative treatment in CMML implying that T cell immunity likely plays a role in eliminating malignant cells. This notion is further supported by the clinical efficacy of donor lymphocyte infusion (DLI) using CD3+ T cells in CMML patients with relapse after allo-SCT. In the present study, we observed that primary CMML cells triggered significant ROS-dependent apoptosis in the CD8+ T cell compartment, in particular in the non-naive cells that are assumed to mediate clearance of leukemic cells. These findings are compatible with an earlier report showing that CD8+T_{EM} are highly sensitive to cell death induced by low levels of exogenous ROS. The mechanisms explaining the differential sensitivity of T cell subsets to CMML cell-derived ROS should be further defined. Also, further studies are required to clarify whether CMML cell-induced immunosuppression towards T cells impacts on the course of disease, and whether ROS formation by leukemic cells may contribute to the reportedly limited benefit of allo-SCT in CMML.

The NOX2 inhibitor HDC has been shown to prevent relapse of AML when used in conjunction with low-dose IL-2 after the completion of chemotherapy. Post-hoc analyses of phase III trial result suggested that the clinical efficacy of HDC/IL-2 is pronounced in patients with AML classes 4 and 5 according to the French-American-British (FAB) classification. These subtypes of AML contain leukemic populations with monocytic differentiation that carry functional NOX2 with preserved capacity to produce and release ROS along with H₂R. These findings have formed the background to the hypothesis that ROS production by monocytic AML cells may serve as a mechanism of leukemia-induced immune escape. The results of the present study imply that similar immunosuppressive mechanisms may be at hand in CMML.

In summary, our results suggest that primary human CMML cells produce immunosuppressive ROS that may impact on NK cell- and T cell-dependent clearance of leukemic cells. We propose that strategies to target extracellular ROS may be of value in ameliorating anti-leukemic cellular immunity in CMML.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method of reducing the tumor burden in a subject with primary or metastatic cancer comprising the step of: administering a therapeutic amount of histamine dihydrochloride and inhibitors of Programmed cell Death protein 1 (PD-1) or Programmed cell Death Ligand 1 (PD-L1) to said subject, thereby reducing the tumor burden in said subject.

2. A method of reducing the risk of metastatic tumor spread in a subject with active cancer comprising the step of: administering a therapeutic amount of histamine dihydrochloride and inhibitors of Programmed cell Death protein 1 (PD-1) or Programmed cell Death Ligand 1 (PD-L1) to said subject, thereby reducing the risk of metastatic tumor spread in said subject.

3. A method of preventing or delaying the reappearance, recurrence or metastatic spread of cancer in a subject comprising the step of: administering a therapeutic amount of histamine dihydrochloride and inhibitors of Programmed cell Death protein 1 (PD-1) or Programmed cell Death Ligand 1 (PD-L1) to said subject, thereby preventing or delaying the reappearance, recurrence or metastatic spread of said cancer in said subject.

4. A method of preventing relapse to a cancer in a subject comprising the step of: administering a therapeutic amount of histamine dihydrochloride and inhibitors of Programmed cell Death protein 1 (PD-1) or Programmed cell Death Ligand 1 (PD-L1) to said subject, thereby preventing relapse to said cancer in said subject.

5. A method of delaying the relapse to a cancer in a subject in remission from said cancer comprising the step of: administering a therapeutic amount of a histamine receptor agonist and inhibitors of Programmed cell Death protein 1 (PD-1) or Programmed cell Death Ligand 1 (PD-L1) to said subject, thereby delaying the relapse to said cancer in said subject in remission from said cancer.

6. A method of prolonging the remission from a cancer in a subject comprising the step of: administering a therapeutic amount of a histamine receptor agonist and inhibitors of Programmed cell Death protein 1 (PD-1) or Programmed cell Death Ligand 1 (PD-L1) to said subject, thereby prolonging the remission from said cancer in said subject.

7. A method of increasing the survival of a subject in remission from a cancer comprising the step of: administering a therapeutic amount of a histamine receptor agonist and inhibitors of Programmed cell Death protein 1 (PD-1) or Programmed cell Death Ligand 1 (PD-L1) to said subject, thereby increasing the survival of said subject.

8. A method of prolonging the survival time of a subject in remission from a cancer comprising the step of: administering a therapeutic amount of a histamine receptor agonist and inhibitors of Programmed cell Death protein 1 (PD-1) or Programmed cell Death Ligand 1 (PD-L1) to said subject, thereby prolonging the survival time of said subject.

9. A method of reducing malignant tumor growth in a subject comprising the step of: administering a therapeutic amount of a histamine receptor agonist and Programmed cell Death protein 1 (PD-1) or Programmed cell Death Ligand 1 (PD-L1) inhibitors to said subject, thereby reducing malignant tumor growth in said subject.

10. A method of decreasing or alleviating cancer symptoms in a subject in remission from a cancer comprising the step of: administering a therapeutic amount of a histamine receptor agonist and inhibitors of Programmed cell Death protein 1 (PD-1) or Programmed cell Death Ligand 1 (PD-L1) to said subject, thereby decreasing or alleviating said cancer symptoms in said subject.

11. The method of any one of claims 1-10, wherein said PD-1 or PD-L1 inhibitor is an antibody.

12. The method of claim 11, wherein said antibody is selected from the group consisting of: Nivolumab, Pembrolizumab, Pidilizumab, BMS 936559, and MPDL328OA.

13. The method of any one of claims 5-12, wherein said histamine receptor agonist is histamine dihydrochloride.

14. The method of any one of claims 5-12, wherein said histamine receptor agonist is N-methyl-histamine or 4-methyl-histamine.

15. The method of any one of claims 1-14, further comprising the step of administering a therapeutic amount of an additional immunostimulant.

16. The method of claim 15, wherein said additional immunostimulant is interleukin-2 (IL-2).

17. The method of any one of claims 1-16, further comprising the step of administering a cancer vaccine.

18. A kit for prolonging remission from a cancer in a subject comprising a) a therapeutic amount of a histamine receptor agonist and inhibitors of Programmed cell Death protein 1 (PD-1) or Programmed cell Death Ligand 1 (PD-L1), and instructions for the use of said kit.

19. The kit of claim 18, wherein said PD-1 or PD-L1 inhibitor is an antibody.

20. The kit of claim 19, wherein said antibody is selected from the group consisting of: Nivolumab, Pembrolizumab, Pidilizumab, BMS 936559, and MPDL328OA.

21. The kit of any one of claims 18-20, wherein said histamine receptor agonist is histamine dihydrochloride.

22. The kit of any one of claims 18-20, wherein said histamine receptor agonist is N-methyl-histamine or 4-methyl-histamine.

23. The kit of any one of claims 18-22, further comprising an immunostimulant.

24. The kit of claim 23, wherein said immunostimulant is interleukin-2 (IL-2).

25. A method of predicting the efficacy of a cancer treatment in a subject comprising the steps of:
a. obtaining a first blood sample from said subject;
b. administering a cycle of a treatment or a proposed treatment for said cancer to said subject;
c. obtaining a second blood sample from said subject after completion of said cycle of treatment; and
d. measuring the frequency of CD8+ cytotoxic T cell phenotypes in said first blood sample and said second blood sample,
wherein if there is a re-distribution of cytotoxic T cells such that there is a reduction in the frequency of T effector memory cells (T_{EM}) and an increase in the frequency of T effector cells (T_{eff}) in said second blood sample compared to said first blood sample, then said cancer treatment is predicted to be effective in said subject and
wherein if there is no re-distribution of cytotoxic T cells in said second blood sample compared to said first blood sample, then said cancer treatment is predicted not to be effective in said subject,
thereby predicting the efficacy of said cancer treatment in said subject.

26. A method of preventing relapse to a cancer in a subject in remission from said cancer comprising the steps of:
a. obtaining a first blood sample from said subject;
b. administering a cycle of a treatment or a proposed treatment for said cancer to said subject;
c. obtaining a second blood sample from said subject after completion of said cycle of treatment;
d. measuring the frequency of CD8+ cytotoxic T cell phenotypes in said first blood sample and said second blood sample, and
e. administering additional cycles of said treatment to said subject if there is a re-distribution of cytotoxic T cells such that there is a reduction in the frequency of T effector memory cells (T_{EM}) and an increase in the frequency of T effector cells (T_{eff}) in said second blood sample compared to said first blood sample,
thereby preventing relapse to said cancer in said subject.

27. A method of prolonging remission from a cancer in a subject comprising the steps of:
a. obtaining a first blood sample from said subject;
b. administering a cycle of a treatment or a proposed treatment for said cancer to said subject;
c. obtaining a second blood sample from said subject after completion of said cycle of treatment;
d. measuring the frequency of CD8+ cytotoxic T cell phenotypes in said first blood sample and said second blood sample, and
e. administering additional cycles of said treatment to said subject if there is a re-distribution of cytotoxic T cells such that there is a reduction in the frequency of T effector memory cells (T_{EM}) and an increase in the frequency of T effector cells (T_{eff}) in said second blood sample compared to said first blood sample,
thereby prolonging remission from said cancer in said subject.

28. The method of any one of claims 1-8, 10-17 and 25-27, wherein said cancer is a leukemia.

29. The method of claim 28, wherein said leukemia is acute myeloid leukemia (AML).

30. The method of any one of claims 1-8, 10-17 and 25-27, wherein said cancer is a thymoma.

31. The method of any one of claims 5-8, 10-17, and 25-30, wherein said subject is in complete remission (CR) from said cancer.

32. The method of any one of claims 25-31, wherein said cancer treatment comprises administration of a cytokine to said subject.

33. The method of claim 32, wherein said cytokine is an interleukin.

34. The method of claim 33, wherein said interleukin comprises IL-2.

35. The method of claim 33, wherein said interleukin comprises IL-12 or IL-15.

36. The method of any one of claims 33-35, wherein said interleukin is administered at low dose.

37. The method of claim 34, wherein said IL-2 is administered at a dosage of 16,400 U/kg twice a day.

38. The method of claim 32, wherein said cytokine comprises an interferon.

39. The method of claim 38, wherein said interferon comprises interferon-alpha.

40. The method of claim 38, wherein said interferon comprises interferon-beta.

41. The method of claim 38, wherein said interferon comprises interferon-gamma.

42. The method of claim 32, wherein said cytokine comprises a hematopoietic growth factor.

43. The method of claim 42, wherein said hematopoietic growth factor is selected from the group consisting of: Erythropoietin, IL-11, Granulocyte-macrophage colony-stimulating factor (GM-CSF), and granulocyte colony-stimulating factor (G-CSF), or a combination thereof.

44. The method of any one of claims 25-43, wherein said cancer treatment comprises administration of a histamine receptor agonist to said subject.

45. The method of claim 44, wherein said histamine receptor agonist is histamine dihydrochloride.

46. The method of claim 44, wherein said histamine receptor agonist is N-methyl-histamine or 4-methyl-histamine.

47. The method of claim 45, wherein the histamine dihydrochloride is administered at 0.5 mg twice a day.

48. The method of any one of claims 25-47, wherein said cancer treatment comprises administration of histamine dihydrochloride and interleukin-2 (IL-2).

49. The method of any one of claims 25-48, wherein said subject in remission is undergoing immunotherapy for relapse prevention.

50. The method of any one of claims 25-49, wherein said cancer treatment comprises administration of inhibitors of Programmed cell Death protein 1 (PD-1) or Programmed cell Death Ligand 1 (PD-L1).

51. The method of any one of claims 25-50, wherein said treatment cycle is the first cycle of said treatment.

52. The method of any one of claims 25-50, wherein said treatment cycle is the third cycle of said treatment.

53. The method of any one of claims 25-52, wherein said cycle is a 3-week cycle of treatment.

54. The method of any one of claims 25-53, wherein said blood sample is a peripheral blood sample.

55. The method of any one of claims 26-54, wherein said treatment or proposed treatment is administered subcutaneously.

56. A method of preventing relapse to acute myeloid leukemia (AML) in a subject with AML in complete remission (CR) comprising the steps of:
a. obtaining a first blood sample from said subject;
b. administering a first cycle of histamine dihydrochloride and interleukin-2 (IL-2) to said subject;
c. obtaining a second blood sample from said subject after completion of said first cycle of treatment;
d. measuring the frequency of CD8+ cytotoxic T cell phenotypes in said blood sample; and
e. administering additional cycles of histamine dihydrochloride and IL-2 to said subject if there is a re-distribution of cytotoxic T cells in said second blood sample compared to said first blood sample such that there is a reduction in the frequency of T effector memory cells (T_{EM}) and an increase in the frequency of T effector cells (T_{eff}),
thereby preventing relapse to AML in said subject.

57. A kit for predicting the efficacy of a cancer treatment in a subject comprising a therapeutic amount of a histamine receptor agonist, an immunostimulant, a means for measuring CD8+ cytotoxic phenotypes, and instructions for the use of said kit.

58. The kit of claim 57, wherein said histamine receptor agonist is histamine dihydrochloride.

59. The kit of claim 57, wherein said histamine receptor agonist is N-methyl-histamine or 4-methyl-histamine.

60. The kit of any one of claims 57-59, wherein said immunostimulant is a cytokine.

61. The kit of claim 60, wherein said cytokine is an interleukin.

62. The kit of claim 61, wherein said interleukin comprises IL-2.

63. The kit of claim 61, wherein said interleukin comprises IL-12 or IL-15.

64. The kit of any one of claims 57-63, wherein said means for measuring CD8+ cytotoxic phenotypes comprises antibodies to CD25, CD69, and IFN-γ.

65. The kit of any one of claims 57-63, wherein said means for measuring CD8+ cytotoxic phenotypes comprises CD3-FITC (HIT3a), CD4-APC-H7 (RPA-T4), CD4-Horizon V450 (RPA-T4), CD8-APC (RPA-T8), CD8-PerCP-Cy5.5 (RPA-T8/SK1), CD8-Qdot705 (3B5), CD16-Horizon V450 (3G8), CD25-Brilliant Violet 421 (M-A251), CD45RA-APC (HI100), CD45RO-PE (UCHL1), CD56-PerCP-eFluor710 (CMSSB), CD56-PE-Cy7 (NCAM16.2), CD69-PE-Cy7 (FN50), HLA-DR-FITC (L243), CCR7-PE-Cy7 (G043H7), CD3-Pacific Blue (S4.1), CD14-Qdot655 (TüK4) and streptavidin-Qdot605, IFN-γ-PE-Cy7 or a combination thereof.

66. The kit of any one of claims 57-65, further comprising one or more containers for collecting blood samples.

67. The kit of any one of claims 57-66, further comprising a therapeutic amount of inhibitors of Programmed cell Death protein 1 (PD-1) or Programmed cell Death Ligand 1 (PD-L1).

68. The kit of claim 67, wherein said PD-1 or PD-L1 inhibitor is an antibody.

69. The kit of claim 68, wherein said antibody is selected from the group consisting of: Nivolumab, Pembrolizumab, Pidilizumab, BMS 936559, and MPDL328OA.

70. A method of predicting the efficacy of a cancer treatment in a subject comprising the steps of:
a. obtaining a first blood sample from said subject;
b. administering a cycle of a treatment or a proposed treatment for said cancer to said subject;
c. obtaining a second blood sample from said subject after completion of said cycle of treatment; and
d. measuring frequency of NK cells in said first blood sample and said second blood sample,
wherein if there is an increase in the frequency of NK cells in said second blood sample compared to said first blood sample, then said cancer treatment is predicted to be effective in said subject and
wherein if there is no increase in the frequency of NK cells in said second blood sample compared to said first blood sample, then said cancer treatment is predicted not to be effective in said subject,
thereby predicting the efficacy of said cancer treatment in said subject.

71. The method of claim 70, wherein said cancer is acute myeloid leukemia.

72. The method of any one of claim 70 or 71, wherein said NK cells are CD56^{bright} NL cells, CD16⁺ NK cells or both.

73. The method of any one of claims 70-72, wherein said NK cell level is measured through detection of an NK cell-associated biomarker.

74. The method of claim 73, wherein said NK cell-associated biomarker is NKp30.

75. The method of claim 73, wherein said NK cell-associated biomarker is NKp46.

76. A method of predicting the efficacy of a cancer treatment in a subject comprising the steps of:
a. obtaining a first blood sample from said subject;
b. administering a cycle of a treatment or a proposed treatment for said cancer to said subject;
c. obtaining a second blood sample from said subject after completion of said cycle of treatment; and
d. measuring the levels of a biomarker expression in said first blood sample and said second blood sample,
wherein if there is an increase in the biomarker expression level in said second blood sample compared to said first blood sample, then said cancer treatment is predicted to be effective in said subject and
wherein if there is no increase in the biomarker expression level in said second blood sample compared to said first blood sample, then said cancer treatment is predicted to be ineffective in said subject,
thereby predicting the efficacy of said cancer treatment in said subject.

77. The method of claim 76, wherein said biomarker is a leukocyte cell surface receptor.

78. The method according to any of the claims 76-77, wherein said biomarker is a monocyte cell surface receptor.

79. The method according to any of the claims 76-78, wherein said biomarker is a histamine type 2 receptor (H2R).

80. A method of predicting the efficacy of a cancer treatment in a subject comprising the steps of:
a. obtaining a first blood sample from said subject;
b. administering a cycle of a treatment or a proposed treatment for said cancer to said subject;
c. obtaining a second blood sample from said subject after completion of said cycle of treatment; and
d. measuring the levels of a biomarker expression in said first blood sample and said second blood sample,
wherein if there is an decrease in the biomarker expression level in said second blood sample compared to said first blood sample, then said cancer treatment is predicted to be effective in said subject and
wherein if there is no decrease in the biomarker expression level in said second blood sample compared to said first blood sample, then said cancer treatment is predicted not to be effective in said subject,
thereby predicting the efficacy of said cancer treatment in said subject.

81. The method of claim 80, said biomarker is a leukocyte cell surface receptor.

82. The method according to any of the claims 80-81, wherein said biomarker is human leukocyte antigen (HLA) A.

83. The method according to any of the claims 80-81, wherein said biomarker is human leukocyte antigen (HLA) B.

84. The method according to any of the claims 80-81, wherein said biomarker is human leukocyte antigen (HLA) C.

85. A method of treating a cancer in a subject comprising the step of: administering a therapeutic amount of an agent that decreases reactive oxygen species (ROS) to said subject.

86. The method of claim 85, wherein said agent decreases extracellular ROS.

87. The method according to any one of the claims 85-86, wherein said agent is an inhibitor of ROS formation.

88. The method according to any one of the claims 85-86, wherein said agent is a scavenger of extracellular ROS.

89. The method according to any one of the claims 85-88, further comprising the step of administering a histamine receptor agonist.

90. The method of claim 89, wherein said histamine receptor agonist is histamine dihydrochloride.

91. The method of claim 89, wherein said histamine receptor agonist is N-methyl-histamine or 4-methyl-histamine.

92. The method of any one of claims 70-91, wherein said cancer treatment comprises administration of histamine dihydrochloride and interleukin-2 (IL-2).

93. The method according to any one of the claims 70-92, wherein said cancer is leukemia.

94. The method of the claim 93, wherein said leukemia is chronic myelomonocytic leukemia (CMML).
